(19) United States
(12) Patent Application Publication
(10) Pub. No.: US 2018/0203008 A1
(43) Pub. Date: Jul. 19, 2018

(54) SENSOR FOR ANALYZING ANALYTE AND METHOD OF ANALYZING ANALYTE

(71) Applicant: PANASONIC HEALTHCARE HOLDINGS CO., LTD., Tokyo (JP)

(72) Inventor: Mie TAKAHASHI, Ehime (JP)

(21) Appl. No.: 15/920,135

(22) Filed: Mar. 13, 2018

Related U.S. Application Data
(63) Continuation of application No. PCT/JP2016/073524, filed on Aug. 10, 2016.

(30) Foreign Application Priority Data
Sep. 28, 2015 (JP) 2015-190226
Sep. 28, 2015 (JP) 2015-190227

(51) Int. Cl. G01N 33/543; B01L 3/00

(52) U.S. Cl. CPC G01N 33/54366; B01L 3/502723; B01L 3/50273; B01L 3/502738; B01L 2300/0816; B01L 2400/0406; B01L 2200/0621; B01L 2300/0867; B01L 2300/047; B01L 2400/0694

(57) ABSTRACT

A sensor includes: a first chamber; a first liquid supply port; an analyte trap; a first exhaust hole; a first flow channel connecting the first liquid supply port, the analyte trap, and the first exhaust hole; a second liquid supply port; a second exhaust hole; and a second flow channel connecting the second liquid supply port, the analyte trap, and the second exhaust hole. The first flow channel and the second flow channel overlap with each other by a predetermined length. In a closed state of the second exhaust hole, a first liquid is drawn into the first flow channel from the first liquid supply port and reaches the analyte trap. In the opened state of the second exhaust hole, a second liquid is drawn into the second flow channel from the second liquid supply port, passes through the analyte trap.

### CROSS-REFERENCE TO RELATED APPLICATIONS

This application is based upon and claims the benefit of priority from the prior Japanese Patent Application No. 2015-190226, filed on Sep. 28, 2015, Japanese Patent Application No. 2015-190227, filed on Sep. 28, 2015, and International Patent Application No. PCT/JP2016/073524, filed on Aug. 10, 2016, the entire content of each of which is incorporated herein by reference.

### BACKGROUND

### Field of the Invention

The present application relates to a sensor for analyzing an analyte, a measurement device, and a method of analyzing an analyte.

### Description of the Related Art

A method of analyzing an analyte using a binding reaction between an analyte which is an object to be analyzed and a ligand which is specifically bound to the analyte has been known. Examples of such an analysis method include an immunoassay method. The immunoassay method includes a competitive immunoassay method and a non-competitive immunoassay method. In addition, conventionally, an automatic immunoassay device that measures an analytical result of an analyte using such a kind of analysis method has been known (for example, see patent document 1).

Patent document 1: JP H08-178927 A

There is a demand for simplification of a structure and easy implementation of analyte measurement in measurement devices that measure an analytical result of an analyte.

### SUMMARY OF THE INVENTION

The present application has been made in view of these circumstances, and an object thereof is to provide a technique for achieving both simplification of a device used for analyte measurement and ease of analyte measurement.

One embodiment of the present application is a sensor for analyzing an analyte. The sensor includes: a substrate; a first chamber positioned inside the substrate; a first liquid supply port which communicates between the first chamber and an outside of the substrate and through which a first liquid containing an analyte flows from the outside of the substrate to the first chamber; an analyte trap positioned inside the first chamber and structured to capture the analyte in the first liquid; a first exhaust hole which communicates between the first chamber and the outside of the substrate and through which a gas inside the first chamber flows to the outside of the substrate; a first flow channel positioned inside the first chamber and connecting the first liquid supply port, the analyte trap, and the first exhaust hole; a second liquid supply port which communicates between the first chamber and an outside of the first chamber and through which a second liquid containing a wash solution of the analyte trap flows from the outside of the first chamber to the first chamber; a second exhaust hole which communicates between the first chamber and the outside of the substrate and is switchable from a closed state to an opened state, and through which the gas inside the first chamber flows to the outside of the substrate in the opened state; and a second flow channel positioned inside the first chamber and connecting the second liquid supply port, the analyte trap, and the second exhaust hole. The first liquid supply port and the first exhaust hole are arranged with the analyte trap interposed therebetween in the first flow channel, and the second liquid supply port and the second exhaust hole are arranged with the analyte trap interposed therebetween in the second flow channel. The first liquid is drawn into the first flow channel from the first liquid supply port along with discharge from the first exhaust hole and reaches the analyte trap in the closed state of the second exhaust hole. The second liquid is drawn into the second flow channel from the second liquid supply port along with discharge from the second exhaust hole, passes through the analyte trap, and removes the first liquid from the analyte trap in the opened state of the second exhaust hole.

Another embodiment of the present application is also a sensor for analyzing an analyte. The sensor includes: a substrate; a first chamber positioned inside the substrate; a first liquid supply port which communicates between the first chamber and an outside of the substrate and through which a first liquid containing an analyte flows from the outside of the substrate to the first chamber; an analyte trap positioned inside the first chamber and structured to capture the analyte in the first liquid; a first exhaust hole which communicates between the first chamber and the outside of the substrate and through which a gas inside the first chamber flows to the outside of the substrate; a first flow channel positioned inside the first chamber and connecting the first liquid supply port, the analyte trap, and the first exhaust hole; a second liquid supply port which communicates between the first chamber and an outside of the first chamber and through which a second liquid containing a wash solution of the analyte trap flows from the outside of the first chamber to the first chamber; a second exhaust hole which communicates between the first chamber and the outside of the substrate and is switchable from a closed state to an opened state, and through which the gas inside the first chamber flows to the outside of the substrate in the opened state; and a second flow channel positioned inside the first chamber and connecting the second liquid supply port, the analyte trap, and the second exhaust hole. The first liquid supply port and the first exhaust hole are arranged with the analyte trap interposed therebetween in the first flow channel, and the second liquid supply port and the second exhaust hole are arranged with the analyte trap interposed therebetween in the second flow channel. The first flow channel and the second flow channel intersect each other at the analyte trap. The first liquid is drawn into the first flow channel from the first liquid supply port along with discharge from the first exhaust hole and reaches the analyte trap in the closed state of the second exhaust hole. The second liquid is drawn into the second flow channel from the second liquid supply port along with discharge from the second exhaust hole, passes through the analyte trap, and removes the first liquid from the analyte trap in the opened state of the second exhaust hole.

### BRIEF DESCRIPTION OF DRAWINGS

Embodiments will now be described, by way of example only, with reference to the accompanying drawings which are meant to be exemplary, not limiting, and wherein like elements are numbered alike in several Figures, in which:

FIGS. 1A, 1B and 1C are schematic views illustrating an example of a sandwich immunoassay method.

FIG. 2 is an exploded perspective view illustrating a schematic structure of a sensor according to a first embodiment.

FIG. 3 is a plan view schematically illustrating an internal structure of the sensor according to the first embodiment when viewed from a cover substrate side.

FIGS. 4A, 4B and 4C are plan views schematically illustrating the internal structure of the sensor according to the first embodiment when viewed from the cover substrate side.

FIG. 5 is a view schematically illustrating an example of an electrode pattern included in the sensor according to the first embodiment.

FIG. 6 is a view schematically illustrating an example of a light-shielding portion provided in the sensor according to the first embodiment.

FIG. 7A is a plan view schematically illustrating an internal structure of a sensor according to Modification 1 when viewed from a cover substrate side. FIG. 7B is an enlarged view of the periphery of a first exhaust hole in FIG. 7).

FIGS. 8A, 8B, 8C, 8D, 8E and 8F are photographs illustrating a state where a first liquid and a second liquid are transferred in the sensor according to Modification 1.

FIG. 9 is a graph illustrating measurement results of TnT in Example 1.

FIG. 10 is a plan view schematically illustrating an internal structure of a sensor according to a second embodiment when viewed from a cover substrate side.

FIGS. 11A, 11B and 11C are plan views schematically illustrating the internal structure of the sensor according to the second embodiment when viewed from the cover substrate side.

FIGS. 12A, 12B, 12C and 12D are photographs illustrating a state where a first liquid and a second liquid are transferred in the sensor according to the second embodiment.

FIG. 13A is an exploded perspective view of a sensor according to a third embodiment. FIG. 13B is an enlarged view of the periphery of an analyte trap in a cross section taken along a line A-A of FIG. 13A.

FIG. 14 is a graph illustrating measurement results of TnT in Example 2.

FIG. 15 is a perspective view illustrating a schematic structure of a sensor according to a fourth embodiment.

FIG. 16 is a plan view schematically illustrating an internal structure of a sensor according to a fifth embodiment when viewed from a cover substrate side.

FIG. 17 is a plan view schematically illustrating an internal structure of a sensor according to a sixth embodiment when viewed from a cover substrate side.

FIGS. 18A and 18B are plan views schematically illustrating the internal structure of the sensor according to the sixth embodiment when viewed from the cover substrate side.

FIGS. 19A and 19B are plan views schematically illustrating a state where a first liquid and a second liquid are transferred in the sensor according to the sixth embodiment.

FIG. 20 is a plan view schematically illustrating an internal structure of a sensor according to a seventh embodiment when viewed from a cover substrate side.

FIGS. 21A and 21B are plan views schematically illustrating the internal structure of the sensor according to the seventh embodiment when viewed from the cover substrate side.

FIGS. 22A and 22B are plan views schematically illustrating a state where a first liquid and a second liquid are transferred in the sensor according to the seventh embodiment.

FIG. 23 is a block diagram schematically illustrating a functional configuration of a measurement device according to an eighth embodiment.

FIG. 24 is an enlarged cross-sectional view illustrating the periphery of a sensor support in the measurement device.

FIG. 25 is an exploded perspective view illustrating a schematic structure of a sensor according to a ninth embodiment.

FIG. 26 is a plan view schematically illustrating an internal structure of the sensor according to the ninth embodiment when viewed from a cover substrate side.

FIGS. 27A to 27C are plan views schematically illustrating the internal structure of the sensor according to the ninth embodiment when viewed from the cover substrate side.

FIG. 28 is a view schematically illustrating an example of an electrode pattern included in the sensor according to the ninth embodiment.

FIG. 29 is a view schematically illustrating an example of a light-shielding portion provided in the sensor according to the ninth embodiment.

FIGS. 30A, 30B, 30C, 30D, 30E and 30F are photographs illustrating a state where a first liquid and a second liquid are transferred in the sensor according to the ninth embodiment.

FIG. 31 is a plan view schematically illustrating an internal structure of a sensor according to a tenth embodiment when viewed from a cover substrate side.

FIGS. 32A, 32B, 32C, 32D, 32E, 32F and 32G are photographs illustrating a state where a first liquid and a second liquid are transferred in the sensor according to the tenth embodiment.

FIG. 33A is an exploded perspective view of a sensor according to an eleventh embodiment. FIG. 33B is an enlarged view of the periphery of an analyte trap in a cross section taken along a line A-A of FIG. 33A.

FIG. 34 is a perspective view illustrating a schematic structure of a sensor according to a twelfth embodiment.

FIG. 35 is a plan view schematically illustrating an internal structure of a sensor according to a thirteenth embodiment when viewed from a cover substrate side.

FIG. 36 is an enlarged cross-sectional view illustrating the periphery of a sensor support in a measurement device according to a fourteenth embodiment.

### DETAILED DESCRIPTION OF THE INVENTION

Hereinafter, the present invention will be described with reference to the drawings based on preferred embodiments. The embodiments are described only for exemplary purposes without limiting the invention, and all features and combinations thereof described in the embodiments are not necessarily essential to the invention.

The inventor of the present application has conducted studies on an analyte analysis method using a binding reaction between an analyte and a ligand and a method of measuring an analysis result. There is a case where prompt analysis and measurement are required depending on a kind of an analyte in these analysis method and measurement method. Examples of the case where the prompt analysis is required include a case where an analyte is a myocardial marker. That is, prompt treatment and action are required for a patient who has developed myocardial infarction. Thus, when there is a suspected myocardial infarction in a patient in the middle of being transported to a hospital, for example, it is possible to perform appropriate treatment during the transport if it is possible to analyze and measure a myocardial marker of the patient and to make a diagnosis based on a result of the analysis and measurement. In addition, the action after being transported to the hospital may be smoothly taken.

Examples of the myocardial marker include troponin I (TnI), troponin T (TnT), creatine kinase MB fraction (CK-MB), brain natriuretic peptide (BNP), myoglobin, and heart type fatty acid-binding protein (H-FABP). When these myocardial markers are analytes, these analytes are generally analyzed by a competitive immunoassay method or a non-competitive immunoassay method.

An operation is complicated and a long time is required when the competitive immunoassay method or the non-competitive immunoassay method is manually carried out. In addition, a measurement device having a large size and a complicated configuration as disclosed in patent document 1 is suitable for simultaneous processing of multiple specimens and processing in a short time, but is hardly mounted to a transport vehicle because the device is large. Therefore, this measurement device is not suitable in the case, such as the myocardial marker, where promptness is required for measurement and mobility (portability) is required for a measurement device.

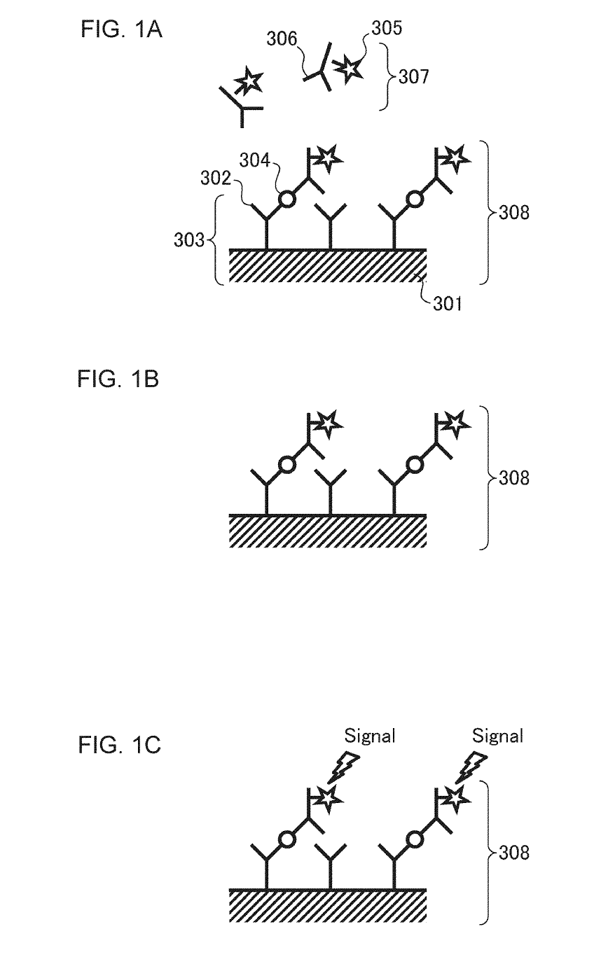

For example, steps illustrated in FIG. 1 are carried out in a sandwich immunoassay method, which is the non-competitive immunoassay method. FIGS. 1(A) to 1(C) are schematic views illustrating an example of the sandwich immunoassay method. A primary antibody 302 (hereinafter referred to as a “solid-phase immobilized antibody 303”) immobilized to a solid phase 301, a specimen sample containing an antigen 304 which is an analyte, and a secondary antibody 306 (hereinafter referred to as a “labeled antibody 307”) to which a label substance 305 has been bound are used in a measurement system exemplified in FIG. 1.

Examples of the solid phase 301 include a magnetic material such as magnetic particles (sometimes referred to as “magnetic beads”, “magnetism particles” or “magnetism beads”, and the like), a well wall surface of a plate made of polystyrene, polycarbonate, or the like, a metal substrate surface, and the like although not particularly limited. Examples of the label substance 305 include enzymes, chemiluminescent substances, bioluminescent substances, electrochemiluminescent substances, fluorescent substances, electron mediators, and the like. It is possible to measure an analyte by acquiring a signal corresponding to the label substance 305 as an analysis result and detecting this signal. The signal varies depending on a type of the label substance 305, and examples thereof include luminescence, fluorescence, absorbance, an electrochemical signal, and the like. Examples of the electrochemical signal include a current, a voltage, and the like.

In the sandwich immunoassay method, first, the solid-phase immobilized antibody 303, the antigen 304, and the labeled antibody 307 are caused to react with each other. As a result, a composite 308 in which the solid-phase immobilized antibody 303 and the labeled antibody 307 are bound to the antigen 304 is generated as illustrated in FIG. 1(A). A reaction solution at this stage contains the labeled antibody 307 and the primary antibody 302 which have not involved in formation of the composite 308, the label substance 305 and the secondary antibody 306 unbound to each other, unnecessary components in the specimen, and substances non-specifically bound to the solid phase 301 or the like (hereinafter these substances are referred to as “unreacted substances”).

The unreacted substance becomes a major factor of lowering analysis sensitivity and analysis accuracy of the antigen 304. Thus, it is necessary to remove the reaction solution from a reaction field to separate a reaction product, that is, the composite 308 and the unreacted substance as illustrated in FIG. 1(B). This separation process is called bound/free separation (B/F separation). The B/F separation includes not only a case of simply removing the reaction solution but also a case of washing the reaction field with a wash solution together with the separation of the reaction solution. It is possible to more reliably separate the reaction product and the unreacted substance by washing with the wash solution. In addition, when the solid phase 301 is a magnetic material, it is necessary to remove unnecessary reaction solution and wash solution in a state where the magnetic material is captured by the magnet.

A substrate configured to generate the signal corresponding to the label substance 305 is introduced after or simultaneously with the B/F separation. As a result, the signal corresponding to the label substance 305 is generated as illustrated in FIG. 1(C). It is possible to measure presence or the amount of the antigen 304 by detecting this signal.

It is necessary to replace the reaction solution with another liquid such as the wash solution in order for the B/F separation. In some cases, it is necessary to replace the wash solution which has been used to remove the reaction solution with another liquid. Thus, when there is an attempt to provide the function of B/F separation in an analyte analysis device, the device tends to be complicated and increased in size. Therefore, such an attempt is not suitable in a case where the promptness is required for analysis and measurement and the portability of the measurement device is required to enable mounting to the transport vehicle such as the myocardial marker. In addition, when there is an attempt to cause a measurer to perform the B/F separation in order to simplify the device, the measurer is forced to perform complicated work so that the ease of analyte measurement is impaired.

Therefore, the inventor of the present application has extensively carried out studies on a configuration in which an analyte can be easily analyzed and measured with a compact device, and has conceived novel sensor, measurement device, and method of analyzing an analyte. The overview of the embodiments of the present application is as follows. In the following embodiments, a sandwich immunoassay method which is a non-competitive immunoassay method will be described as the analyte analysis method to be executed using the sensor and measurement device. However, the invention is not limited thereto, and the sensor and measurement device according to the embodiments can be applied generally to analyte analysis methods that require the B/F separation.

Examples of the analyte analysis method that require the B/F separation can include not only the competitive and non-competitive immunoassay methods but also a gene detection method using hybridization, and the like. Therefore, the term “ligand” used in this specification refers to a substance that is specifically bound to an analyte, and is not limited to an antibody used in the competitive and non-competitive immunoassay methods. Examples of the ligand include antigens, binding proteins, DNA, RNA, and the like. In addition, “analyte analysis” in this specification means to generate a signal or acquire a signal using a sensor 1. In addition, “analyte measurement” means to detect the generated or acquired signal using a measurement device 200.

### Sensor for Analyzing Analyte

Hereinafter, a sensor for analyzing an analyte will be described by exemplifying first to seventh embodiments, Modifications 1 and 2, and Examples 1 and 2.

### First Embodiment

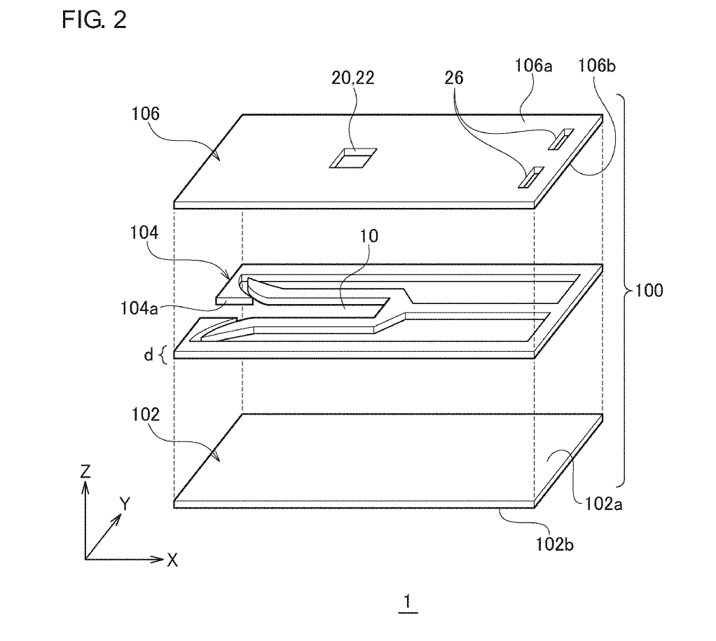

FIG. 2 is an exploded perspective view illustrating a schematic structure of a sensor according to the first embodiment. The sensor 1 according to the present embodiment is a sensor that analyzes an analyte and has a substrate 100. The substrate 100 includes a base substrate 102, a spacer member 104, and a cover substrate 106. The spacer member 104 is arranged on a surface of the base substrate 102. The cover substrate 106 is arranged on a surface of the spacer member 104 on a side opposite to the base substrate 102 side. The substrate 100 is formed by stacking the base substrate 102, the spacer member 104, and the cover substrate 106 in this order, and bonding these substrates to each other with an adhesive or the like.

Incidentally, the base substrate 102 and the spacer member 104 may be integrally formed, and the cover substrate 106 may be bonded to these base substrate 102 and spacer member 104, for example. In addition, the spacer member 104 and the cover substrate 106 may be integrally formed, and the base substrate 102 may be bonded to the spacer member 104 and the cover substrate 106. In addition, for example, a member formed using a resin material such as polyethylene terephthalate (PET), polystyrene, polycarbonate, and acrylic can be adopted as the base substrate 102, the spacer member 104, and the cover substrate 106. In addition, a substrate formed using glass may be adopted as the base substrate 102 and the cover substrate 106. The respective substrates and member are attached to each other by, for example, an adhesive such as a hot-melt paste and a UV curable paste, or an adhesive tape. In this case, the spacer member 104 may be configured directly using the adhesive or the adhesive tape. That is, the spacer member 104 in the present application includes the adhesive or the adhesive tape. Alternatively, the respective substrates and member may be attached to each other by an ultrasonic welding method.

The base substrate 102 has a flat plate shape and has a first main surface 102a and a second main surface 102b opposite to the first main surface 102a. The spacer member 104 is stacked on the first main surface 102a.

The spacer member 104 is a planar member having a predetermined thickness d in a stacking direction (a Z-axis direction in FIG. 2) of the base substrate 102, the spacer member 104, and the cover substrate 106. In addition, the spacer member 104 has a slit 104a extending in a plane direction (XY directions in FIG. 2) of the spacer member 104. The slit 104a passes through the spacer member 104 in a direction of the thickness d. That is, the spacer member 104 has a shape in which a part of the flat plate is cut out by the slit 104a.

The cover substrate 106 has a flat plate shape and has a first main surface 106a and a second main surface 106b which is opposite to the first main surface 106a. The cover substrate 106 is stacked on the spacer member 104 such that the second main surface 106b faces the spacer member 104 side. The cover substrate 106 is provided with a first exhaust hole 20, a second liquid supply port 22, a second exhaust hole 26, and the like.

In the substrate 100, a first chamber 10 is provided. The first chamber 10 is formed by the first main surface 102a of the base substrate 102, the second main surface 106b of the cover substrate 106, and the slit 104a. That is, the first main surface 102a of the base substrate 102 defines a lower surface of the first chamber 10. A wall surface of the slit 104a of the spacer member 104 defines a side surface of the first chamber 10. The second main surface 106b of the cover substrate 106 defines an upper surface of the first chamber 10. Therefore, the first chamber 10 is a space defined by the base substrate 102, the spacer member 104, and the cover substrate 106.

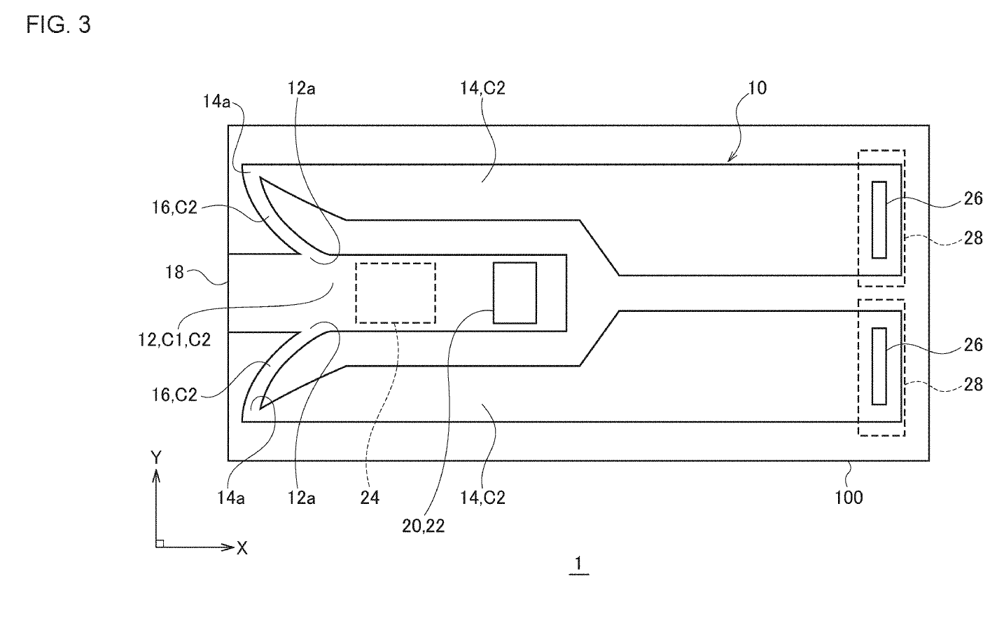

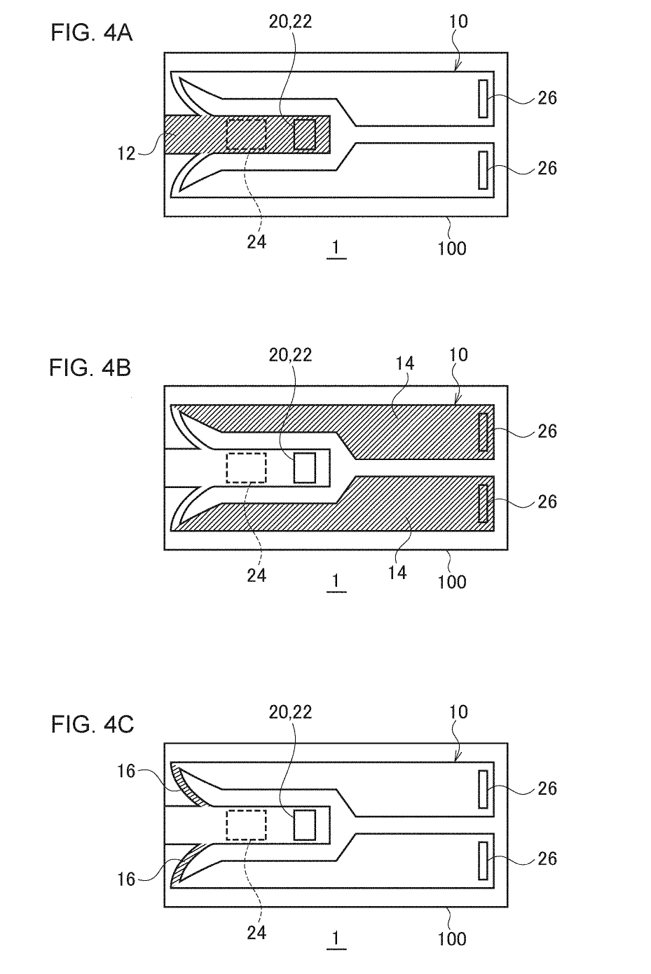

FIGS. 3 and 4(A) to 4(C) are plan views schematically illustrating an internal structure of the sensor 1 according to the first embodiment when viewed from the cover substrate 106 side. For convenience of description, the first exhaust hole 20, the second liquid supply port 22, and the second exhaust hole 26 provided in the cover substrate 106 are also illustrated in FIGS. 3 and 4(A) to 4(C).

The first chamber 10 is arranged inside the substrate 100. The first chamber 10 includes a first part 12, a second part 14, and a coupler 16 connecting the first part 12 and the second part 14. The first part 12 is a hatched region in FIG. 4(A). The second part 14 is a hatched region in FIG. 4(B). The coupler 16 is a hatched region in FIG. 4(C). The sensor 1 according to the present embodiment has one first part 12, two second parts 14, and two couplers 16. The two second parts 14 are arranged with the first part 12 interposed therebetween, and the first part 12 and each of the second part 14 are coupled by the coupler 16. A combination of one of the second parts 14 and the coupler 16 and a combination of the other second part 14 and the coupler 16 are arranged to be substantially symmetric with each other with the first part 12 interposed therebetween.

The coupler 16 has one end portion connected to the first part 12, extends in a direction intersecting an extending direction of the first part 12, and has the other end portion connected to the second part 14. That is, the coupler 16 and the second part 14 are spaces branching and extending from the first part 12. The number of each of the first part 12, the second part 14 and the coupler 16 is not limited, and the sensor 1 may have at least each one of the first part 12, the second part 14, and the coupler 16 (see the second embodiment to be described later).

In addition, the sensor 1 includes a first liquid supply port 18, the first exhaust hole 20, the second liquid supply port 22, an analyte trap 24, and the second exhaust hole 26. The first liquid supply port 18 is a through-hole that communicates between the first chamber 10 and an outside of the substrate 100. More specifically, the first liquid supply port 18 communicates between the first part 12 of the first chamber 10 and the outside of the substrate 100. In the present embodiment, the slit 104a extends to an outer surface (a side surface connecting the two main surfaces) of the spacer member 104, thereby forming the first liquid supply port 18. A first liquid containing an analyte is spotted to the first liquid supply port 18. As a result, the first liquid flows from the outside of the substrate 100 to the first chamber 10 via the first liquid supply port 18.

The first exhaust hole 20 is a through-hole that communicates between the first chamber 10 and the outside of the substrate 100. More specifically, the first exhaust hole 20 communicates between the first part 12 and the outside of the substrate 100. In the present embodiment, the first exhaust hole 20 is configured using the through-hole extending from the first main surface 106a to the second main surface 106b of the cover substrate 106. A gas in the first chamber 10 can flow to the outside of the substrate 100 via the first exhaust hole 20.

The second liquid supply port 22 is a through-hole that communicates between the first chamber 10 and the outside of the first chamber 10. More specifically, the second liquid supply port 22 communicates between the first part 12 and the outside of the first chamber 10. In the present embodiment, the second liquid supply port 22 communicates between the first chamber 10 and the outside of the substrate 100. In addition, the second liquid supply port 22 also serves as the first exhaust hole 20. That is, the through-hole provided in the cover substrate 106 also serves as the first exhaust hole 20 and the second liquid supply port 22. A second liquid containing a wash solution of the analyte trap 24 is spotted to the second liquid supply port 22. As a result, the second liquid flows from the outside of the first chamber 10 to the first chamber 10 via the second liquid supply port 22. Incidentally, the outside of the first chamber 10 to which the second liquid supply port 22 is connected may be another chamber provided inside the substrate 100. That is, the second liquid supply port 22 may communicate between the first chamber 10 and the other chamber in the substrate 100 (see a fifth embodiment to be described later).

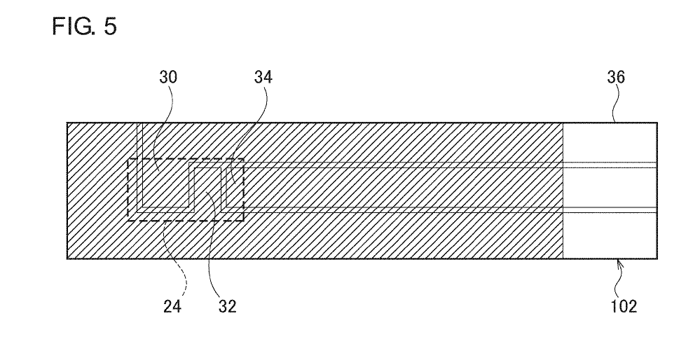

The analyte trap 24 is a region which is positioned inside the first chamber 10 and by which the analyte in the first liquid is captured. More specifically, the analyte trap 24 is positioned in the first part 12. For example, the analyte trap 24 corresponds to the solid phase 301, and the primary antibody 302 is immobilized to the surface of the base substrate 102 forming the analyte trap 24. Alternatively, when the solid phase 301 is made of a magnetic material, an analyte bound to the magnetic material is captured by the analyte trap 24 by a magnetic force of a magnet arranged in the vicinity of the analyte trap 24 (incidentally, a magnetic material to which the analyte is not bound is also captured by the analyte trap 24). In the analyte trap 24, the above-described signal of the label substance 305 is generated. That is, the analyte trap 24 corresponds to an analyte acquisition portion. When the label substance 305 is an electron mediator, at least a working electrode and a counter electrode are arranged in the analyte trap 24 (see FIG. 5).

The second exhaust hole 26 is a through-hole that communicates between the first chamber 10 and the outside of the substrate 100. More specifically, the second exhaust hole 26 communicates between the second part 14 of the first chamber 10 and the outside of the substrate 100. In the present embodiment, the second exhaust hole 26 is configured using the through-hole extending from the first main surface 106a to the second main surface 106b of the cover substrate 106. The second exhaust hole 26 can be switched from a closed state to an opened state. The gas in the first chamber 10 can flow to the outside of the substrate 100 via the second exhaust hole 26 in the opened state.

In the present embodiment, the sensor 1 is provided with a sealing member 28 that closes the second exhaust hole 26. The sealing member 28 is configured using, for example, an adhesive tape or the like and is provided on the first main surface 106a of the cover substrate 106 so as to cover the second exhaust hole 26. It is possible to switch the second exhaust hole 26 from the closed state to the opened state by removing this sealing member 28 or by making a hole in the sealing member 28.

Incidentally, the second exhaust hole 26 may be closed as the material forming the cover substrate 106 is present inside the second exhaust hole 26. That is, the second exhaust hole 26 may be closed by a part of the cover substrate 106. The part of the cover substrate 106 positioned inside the second exhaust hole 26 corresponds to the sealing member 28. This part may be integrated with another part around the second exhaust hole 26. In this case, for example, the user opens a hole in a formation region of the second exhaust hole 26 of the cover substrate 106 at the timing of generating a capillary force in a second flow channel C2, thereby opening the second exhaust hole 26. The cover substrate 106 is preferably subjected to processing to facilitate the formation of the second exhaust hole 26, such as making a thickness of a position where the second exhaust hole 26 is formed thinner than a thickness of the other region.

In the first chamber 10, a first flow channel C1 connecting the first liquid supply port 18, the analyte trap 24, and the first exhaust hole 20 is provided. More specifically, the first flow channel C1 is arranged in the first part 12. That is, a region of the first part 12 from the first liquid supply port 18 to the first exhaust hole 20 forms the first flow channel C1. The first flow channel C1 is a space extending from the first liquid supply port 18 to the first exhaust hole 20. The first liquid supply port 18 and the first exhaust hole 20 are arranged with the analyte trap 24 interposed therebetween in the first flow channel C1. The first liquid supply port 18 is arranged on the opposite side of the first exhaust hole 20 with a position 12a in the first part 12 to which the coupler 16 is connected as a reference.

When the first liquid is supplied to the first liquid supply port 18 in a state where the first liquid supply port 18 and the first exhaust hole 20 are opened and the second exhaust hole 26 is closed, the first liquid is drawn into the first flow channel C1 from the first liquid supply port 18 along with discharge from the first exhaust hole 20. Then, the first liquid reaches the analyte trap 24 and further moves to the first exhaust hole 20. That is, the first liquid moves through the first flow channel C1 due to a capillary phenomenon, reaches the analyte trap 24, and is further drawn to the first exhaust hole 20. When the liquid is spotted to the first exhaust hole 20, the liquid moves toward the first liquid supply port 18.

The first liquid supply port 18 is arranged on a side surface of the substrate 100 in the present embodiment. Thus, the first liquid is spotted from the side of the sensor 1 (the X-axis direction in FIG. 3) to the first liquid supply port 18. Incidentally, the present invention is not particularly limited to this configuration. For example, the base substrate 102 or the cover substrate 106 may be provided with a through-hole communicating between the first chamber 10 and the outside of the substrate 100, and the first liquid supply port 18 may be configured using this through-hole. In this case, the first liquid is spotted to the first liquid supply port 18 from the lower side or the upper side the sensor 1 (the Z-axis direction in FIG. 2).

A size and a shape of the first liquid supply port 18 are not particularly limited as long as having an opening diameter that allows the first liquid spotted to the first liquid supply port 18 to move into the first chamber 10 by the capillary force. A size and a shape of the first flow channel C1 are not particularly limited as long as having a cross-sectional area that allows generation of the above-described capillary force. A size and a shape of the first exhaust hole 20 are not particularly limited as long as having an opening diameter that allows air to move from the first chamber 10 to the outside of the substrate 100.

The first liquid is not particularly limited as long as being a liquid containing at least an analyte. For example, the first liquid is a specimen solution collected from a human body such as blood or urine. In addition, the first liquid may be a liquid obtained by performing predetermined pretreatment to this specimen solution, or a mixture of this specimen solution and a reagent or the like.

The second flow channel C2 connecting the second liquid supply port 22, the analyte trap 24, and the second exhaust hole 26 is provided inside the first chamber 10. More specifically, the second flow channel C2 is arranged across the first part 12, the coupler 16, and the second part 14. That is, the second flow channel C2 is constituted by a region to the position 12a to which the coupler 16 is connected from the second liquid supply port 22 in the first part 12, the coupler 16, and a region to the second exhaust hole 26 from a position 14a in the second part 14 to which the coupler 16 is connected. The second flow channel C2 is a space extending from the second liquid supply port 22 to the second exhaust hole 26. Therefore, the first flow channel C1 and the second flow channel C2 overlap with each other in the region between the second liquid supply port 22 and the position 12a in the first part 12.

The second liquid supply port 22 and the second exhaust hole 26 are arranged with the analyte trap 24 interposed therebetween in the second flow channel C2. In addition, the second liquid supply port 22 is arranged on a side opposite to the first liquid supply port 18 with the position 12a in the first part 12 to which the coupler 16 is connected as a reference. Further, the analyte trap 24 is arranged between the position 12a in the first part 12 and a position where the second liquid supply port 22 is provided, that is, the position to which the second liquid supply port 22 is connected in a direction in which the second liquid flows in the second flow channel C2 (substantially parallel to the X axis in FIG. 3).

When the second liquid is supplied to the second liquid supply port 22 in a state where the first liquid supply port 18 is closed and the second exhaust hole 26 is opened, the second liquid is drawn into the second flow channel C2 from the second liquid supply port 22 along with discharge from the second exhaust hole 26. Then, the second liquid passes through the analyte trap 24 and moves to the second exhaust hole 26 side. That is, the second liquid moves through the second flow channel C2 due to a capillary phenomenon, passes through the analyte trap 24, and reaches the second part 14 via the coupler 16. The second liquid reaching the second part 14 is transferred to the second exhaust hole 26. As the second liquid passes through the analyte trap 24, the first liquid can be removed from the analyte trap 24. The first liquid is drawn into the second part 14 together with the second liquid.

The first liquid supply port 18 is closed as the first liquid is spotted to the first liquid supply port 18. That is, the first liquid supply port 18 is blocked by the first liquid. In addition, the second exhaust hole 26 is switched to the opened state by removing or perforating the sealing member 28 in the present embodiment. Thus, it is possible to easily control the timing at which the capillary force is generated in the second flow channel C2 to draw the second liquid into the second part 14.

In the present embodiment, the second liquid supply port 22 is arranged on the cover substrate 106. Thus, the second liquid is spotted to the second liquid supply port 22 from the upper side of the sensor 1 (the Z-axis direction in FIG. 2). Incidentally, the present invention is not particularly limited to this configuration. For example, the base substrate 102 may be provided with a through-hole communicating between the first chamber 10 and the outside of the substrate 100, and the second liquid supply port 22 may be configured using this through-hole. In this case, the second liquid is spotted to the second liquid supply port 22 from the lower side of the sensor 1 (the Z-axis direction in FIG. 2). In addition, the second liquid supply port 22 may be provided on a side surface of the substrate 100 similarly to the first liquid supply port 18. Similarly, the first exhaust hole 20 and the second exhaust hole 26 may be provided on the side surface of the substrate 100 or the base substrate 102.

A size or a shape of the second liquid supply port 22 are not particularly limited as long as having an opening diameter that allows the second liquid spotted to the second liquid supply port 22 to move into the first chamber 10 by the capillary force. A size and a shape of the second flow channel C2 are not particularly limited as long as having a cross-sectional area that allows generation of the above-described capillary force. A size and a shape of the second exhaust hole 26 are not particularly limited as long as having an opening diameter that allows air to move from the first chamber 10 to the outside of the substrate 100.

The second exhaust holes 26 provided in the two second parts 14 can be independently switched from the closed state to the opened state. Therefore, when only one of the second exhaust holes 26 is opened, the first liquid and the second liquid are drawn into only the second part 14 on the side where the second exhaust hole 26 is opened. When both of the second exhaust holes 26 are opened, each part of the first liquid and the second liquid is drawn into one of the second parts 14, and the other parts of the first liquid and the second liquid are drawn into the other second part 14.

The second liquid is a liquid containing the wash solution to be used in B/F separation. Examples of the wash solution can include an aqueous solvent containing a surfactant. The surfactant used for the wash solution is preferably one that does not affect a reaction such as an antigen-antibody reaction. Examples of such a surfactant can include a non-ionic surfactant. Examples of the non-ionic surfactant include a TWEEN (registered trademark)-based surfactant (polyoxyethylene sorbitan fatty acid esters), and a TRITON (registered trademark)-based surfactant (polyoxyethylene p-t-octylphenyl ethers). In addition, the second liquid may contain a substrate to generate the signal corresponding to the label substance 305 as well as the wash solution. For example, when the analyte measurement system is a system that measures chemiluminescence or bioluminescence as a signal, the second liquid may contain a luminescent substrate, such as a luminol type and a dioxetane type, together with the wash solution. In addition, when the analyte measurement system is a system that measures electrochemiluminescence as a signal, the second liquid may contain an electron mediator, such as tripropylamine (TPA), together with the wash solution.

In addition, when the analyte measurement system is a system that measures an electrochemical signal, the second liquid may contain an electron mediator, such as potassium ferricyanide and a quinone compound, together with the wash solution. In addition, when the analyte measurement system is a system that measures an absorbance, that is, a dye as a signal, the second liquid may contain a chromogenic substrate together with the wash solution. Incidentally, the term “electron mediator” in the present specification refers to a substance that serves as a medium for exchange of electrons in an oxidation-reduction reaction. The electron mediator may be an oxidant or a reductant depending on a signal measurement system.

A sum of volumes of all the second parts 14 and volumes of all the couplers 16 (hereinafter referred to as a total volume A) is desirably larger than a sum of a volume of the analyte trap 24 in the first part 12 and a volume between the first exhaust hole 20 and the analyte trap 24 in the first part 12 (hereinafter referred to as a total volume B). That is, when each number of the second parts 14 and the couplers 16 is N (N is an integer of one or more) in the sensor 1, the total volume A of volumes of the N second parts 14 and volumes of the N couplers 16 is desirably larger than the total volume B of the volume of the analyte trap 24 in the first part 12 and the volume between the first exhaust hole 20 and the analyte trap 24 in the first part 12. It is necessary to replace the first liquid existing in the analyte trap 24 with the second liquid in the B/F separation. Thus, it is possible to reliably replace the first liquid existing in the analyte trap 24 with the second liquid by setting the total volume A and the total volume B to have the above-described relationship.

At least a part of the wall surface inside the first chamber 10, for example, at least one of the first main surface 102a of the base substrate 102, the wall surface of the slit 104a of the spacer member 104, and the second main surface 106b of the cover substrate 106, and the first liquid supply port 18, the second liquid supply port 22, and the like may be subjected to predetermined hydrophilic treatment. It is possible to increase the capillary force generated in the first flow channel C1 or the second flow channel C2 by performing the hydrophilic treatment, and the liquid can be smoothly or reliably transferred due to the capillary phenomenon. Examples of the hydrophilic treatment can include application of a non-ionic, cationic, anionic, or amphoteric surfactant to the wall surface of the first chamber 10 or the liquid supply port, corona discharge treatment, and the like. Examples of the hydrophilic treatment can include formation of a fine uneven structure on the wall surface of the first chamber 10 or a surface of the liquid supply port, and the like (for example, see JP 2007-3361 A).

Next, a description will be given regarding the configuration of the sensor 1 in accordance with an analyte measurement method to be used, that is, a type of a signal to be measured. Each component of the sensor 1 according to the present embodiment can be changed in accordance with the analyte measurement method to be adopted.

### Electrochemical Signal Measurement System

When the analyte measurement system is a system that measures an electrochemical signal such as a current and a voltage, the label substance 305 in the labeled antibody 307 is, for example, an oxidoreductase. In this case, the sensor 1 acquires the electrochemical signal from an electron mediator through which electrons are exchanged by an oxidation-reduction reaction using the oxidoreductase. Alternatively, the sensor 1 acquires the electrochemical signal from hydrogen peroxide. The sensor 1 acquires these electrochemical signals using an electrode. In addition, the label substance 305 is, for example, an electron mediator such as ferrocene. In this case, for example, a current amplified by redox cycling is detected as the electrochemical signal, and the sensor 1 acquires this electrochemical signal by using the electrode.

FIG. 5 is a view schematically illustrating an example of an electrode pattern included in the sensor 1 according to the first embodiment. When the sensor 1 is used in the system that measures the electrochemical signal, at least the first main surface 102a of the base substrate 102 has an insulating property. Then, the sensor 1 has a working electrode 30 and a counter electrode 32 in a region corresponding to the analyte trap 24 of the base substrate 102. Not only the working electrode 30 and the counter electrode 32 but also a reference electrode 34 is provided in the present embodiment.

In addition, the sensor 1 has a connection portion 36 electrically connected to the measurement device. As the sensor 1 is electrically connected to the measurement device, the voltage or current for acquisition of the electrochemical signal is applied from the measurement device to the sensor 1. As this voltage or current is applied to the sensor 1, the electrochemical signal acquired by the sensor 1 through analyte analysis is measured by the measurement device. In FIG. 5, a hatched region is a region where the spacer member 104 and the cover substrate 106 are stacked. A region without hatching positioned at an end portion of the base substrate 102 is an exposed region of the base substrate 102. Each part of the working electrode 30, the counter electrode 32, and the reference electrode 34 is exposed in the exposed region. This exposed region forms the connection portion 36.

Examples of a material of the electrode include a metal material such as gold, platinum, and palladium, a carbon paste, or the like. The electrode can be formed on the base substrate 102, for example, as follows. That is, it is possible to form the electrode by forming a thin film having an electrode pattern shape on the first main surface 102a of the base substrate 102 by sputtering of a metal material. Alternatively, it is possible to form the electrode by performing laser cutting or the like to the thin film stacked on the first main surface 102a Alternatively, it is possible to form the electrode by printing a carbon paste having an electrode pattern shape on the first main surface 102a. Incidentally, the electrode and the connection portion 36 may be provided on the cover substrate 106.

### Electrochemiluminescence Measurement System

When the analyte measurement system is the system that measures electrochemiluminescence, the label substance 305 is an electrochemiluminescent body such as a ruthenium complex and an osmium complex. In this case, the sensor 1 acquires the luminescence of the electrochemiluminescent body, generated as a predetermined voltage is applied in the presence of an electron mediator such as TPA, as a signal. The sensor 1 has an electrode structure similar to that of the case of being used in the electrochemical signal measurement system. Incidentally, the electrochemiluminescence measurement system, the luminescence from the electrochemiluminescent body is measured on the cover substrate 106 side by the measurement device. Thus, at least a portion of the cover substrate 106 corresponding to the analyte trap 24 needs to have a light-transmitting property. Incidentally, the electrode and the connection portion 36 may be provided on the cover substrate 106, and luminescence may be measured on the base substrate 102 side. In this case, at least the portion of the base substrate 102 corresponding to the analyte trap 24 has a light-transmitting property.

### Chemiluminescence/Bioluminescence Measurement System

When the analyte measurement system is the system that measures chemiluminescence or bioluminescence, the label substance 305 is an enzyme such as peroxidase, alkaline phosphatase, and luciferase. In this case, as a chemiluminescent substrate is introduced into the analyte trap 24, a luminescent signal is generated from the chemiluminescent substrate by the label substance 305 existing in the analyte trap 24, that is, the enzyme. Incidentally, a chemiluminescent substance may be used as the label substance 305 instead of the enzyme, and the enzyme may be introduced into the analyte trap 24. In addition, a luminescent system that does not use enzymes, such as a luminescent system that generates a luminescent signal by a combination of a chemiluminescent substance and a luminescent catalytic substrate, may be adopted.

The luminescent signal acquired by the sensor 1 is measured on the base substrate 102 side or the cover substrate 106 side by the measurement device. Thus, a portion of the substrate on a side where the luminescence signal is measured corresponding to the analyte trap 24 needs to have a light-transmitting property. On the other hand, when a portion other than the portion corresponding to the analyte trap 24 also has the light-transmitting property, an unnecessary luminescent signal is measured so that the accuracy in measurement of the analyte is likely to decrease.

That is, the enzyme generates the luminescent signal immediately upon contact with the chemiluminescent/bioluminescent substrate. In addition, the chemiluminescent substance immediately generates the luminescent signal upon contact with the luminescent catalytic substrate. Thus, when the second liquid containing the luminescent substrate is supplied from the second liquid supply port 22 and drawn into the second exhaust hole 26 side after the first liquid reaches the analyte trap 24, the luminescent signal can also be generated from the luminescent substrate that has moved to be closer to the first liquid supply port 18 side or the second exhaust hole 26 side than the analyte trap 24. When the whole substrate on the side where a photodetector of the measurement device is arranged has the light-transmitting property, a luminescent signal generated in a region other than the analyte trap 24 is also measured. Since such a luminescent signal becomes noise, there is a risk that the accuracy in measurement of the analyte may decrease.

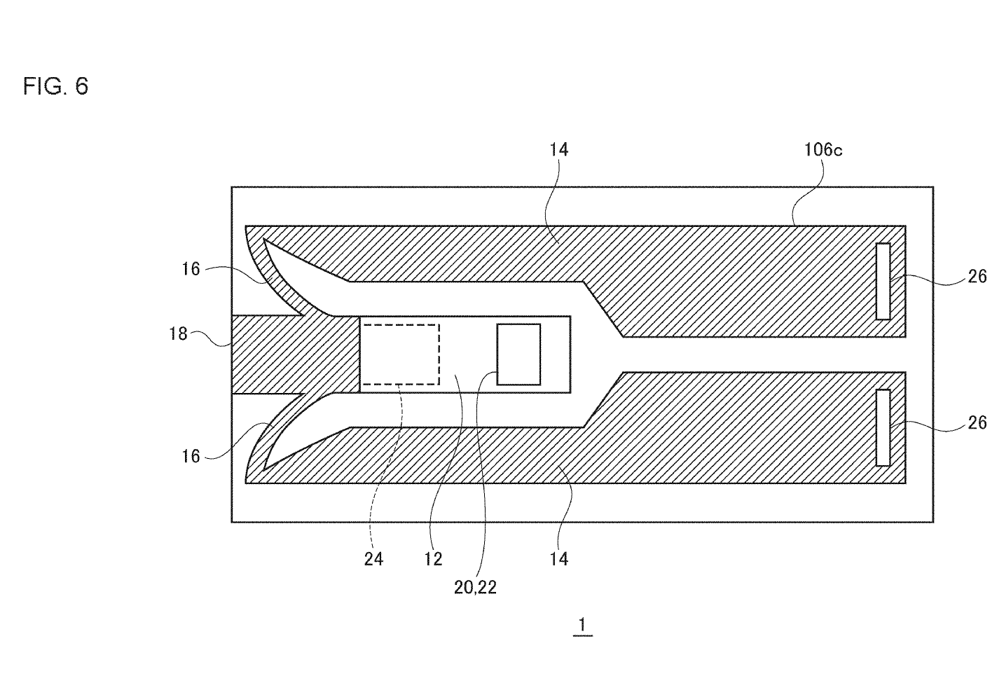

On the other hand, the substrate on the side where the photodetector is arranged has a light-shielding portion 106c in at least a partial region other than the portion corresponding to the analyte trap 24 in the sensor 1 according to the present embodiment. FIG. 6 is a view schematically illustrating an example of the light-shielding portion 106c of the sensor 1 according to the first embodiment. FIG. 6 illustrates the sensor 1 in the case where the cover substrate 106 includes the light-shielding portion 106c as an example.

As illustrated in FIG. 6, the sensor 1 has light-transmitting portions in a portion overlapping with the analyte trap 24 and a portion overlapping with a region closer to the second liquid supply port 22 side than the analyte trap 24 in the first part 12. Then, the light-shielding portion 106c is provided in the other portions, that is, portions overlapping with a region of the first part 12 closer to the first liquid supply port 18 side than the analyte trap 24, the coupler 16, and the second part 14. It is possible to suppress the luminescence signal serving as a noise source from being emitted to the outside of the substrate 100 by providing the light-shielding portion 106c. Incidentally, it is more preferable that the light-shielding portion 106c be provided in the entire portion except for the portion overlapping with the analyte trap 24.

### Fluorescence Measurement System

In the case where the analyte measurement system is a system that measures fluorescence, the label substance 305 is, for example, a fluorescent substance. In this case, the sensor 1 acquires fluorescence generated by irradiation of the fluorescent substance with excitation light as a signal. The label substance 305 is, for example, an enzyme such as alkaline phosphatase. In this case, for example, a fluorescent substrate such as 4-methylumbelliferyl phosphate is introduced, and the fluorescent substance obtained by a reaction of the fluorescent substrate and the enzyme is irradiated with excitation light, whereby fluorescence as a signal is generated.

Examples of the configuration of measuring the fluorescence signal can include a configuration in which excitation light is emitted from the base substrate 102 side to measure a fluorescence signal on the base substrate 102 side, and a configuration in which excitation light is emitted from the cover substrate 106 side to measure a fluorescent signal from the cover substrate 106 side. In this case, a substrate on a side where the irradiation of the excitation light and the measurement of the fluorescence signal are performed is configured such that at least a portion corresponding to the analyte trap 24 is made of a translucent material capable of transmitting the excitation light and the fluorescent signal therethrough.

In addition, a configuration in which excitation light is emitted from one substrate side between the base substrate 102 and the cover substrate 106 to measure a fluorescence signal on the other substrate side can be exemplified as another configuration of measuring the fluorescence signal. In this case, the substrate on the side where the excitation light is emitted is configured such that at least a portion corresponding to the analyte trap 24 is made of a translucent material capable of transmitting the excitation light. In this case, the substrate on the side where the fluorescence signal is measured is configured such that at least a portion corresponding to the analyte trap 24 is made of a translucent material capable of transmitting the fluorescent signal.

### Absorbance Measurement System

When the analyte measurement system is a system that measures an absorbance, the label substance 305 is, for example, an enzyme such as peroxidase or diaphorase. In this case, a chromogenic substrate is introduced into the analyte trap 24, and the chromogenic substrate and the enzyme react with each other so that a dye is generated from the chromogenic substrate. As the dye is irradiated with light having a predetermined wavelength, the absorbance as a signal is obtained.

Examples of a configuration of measuring the absorbance can include a configuration in which light having a predetermined wavelength is emitted from one substrate side between the base substrate 102 and the cover substrate 106 and the transmitted light is measured from the other substrate side. In this case, the base substrate 102 and the cover substrate 106 is configured such that at least a portion corresponding to the analyte trap 24 is made of a translucent material capable of transmitting the emitted light.

In addition, a configuration in which light having a predetermined wavelength is emitted from the base substrate 102 side and the reflected light is measured on the base substrate 102 side, and a configuration in which light having a predetermined wavelength is emitted from the cover substrate 106 side and the reflected light is measured on the cover substrate 106 side can be exemplified as other configurations of measuring the absorbance. In this case, the substrate on the side where the irradiation of light and the measurement of the reflected light are performed is configured such that at least a portion corresponding to the analyte trap 24 is made of a translucent material capable of transmitting the emitted light.

The sensor 1 according to the present embodiment can be used in any of a method of immobilizing the primary antibody 302 to a surface of any substrate corresponding to the analyte trap 24 and a method of immobilizing the primary antibody 302 to a magnetic material regardless of the analyte measurement system. That is, the substrate may be used as the solid phase 301, or the magnetic material may be used as the solid phase 301.

When the metal substrate is used as the solid phase 301, the primary antibody 302 can be immobilized to the surface of the substrate by, for example, a self-assembled monolayer (SAM). Other immobilizing methods include physical adsorption, chemical bonding, and the like. When the magnetic material is used as the solid phase 301, a magnet configured to capture the magnetic material in the analyte trap 24 is arranged in the vicinity of the analyte trap 24. The magnet is arranged, for example, on the second main surface 102b side of the base substrate 102 or on the first main surface 106a side of the cover substrate 106. Incidentally, the magnet may be provided in the sensor 1 or may be provided in the measurement device of the signal acquired by the sensor 1.

Incidentally, the magnet is preferably arranged on a substrate side opposite to a side on which the luminescence is measured when the magnetic material is used as the solid phase 301 in the electrochemiluminescence measurement system or the chemiluminescence/bioluminescence measurement system.

In addition, the magnet is preferably arranged on a substrate side opposite to a side on which the irradiation of the excitation light and the measurement of the fluorescence signal are performed when the fluorescence measurement system has the configuration in which the irradiation of the excitation light and the measurement of the fluorescence signal are performed on the same substrate side and the magnetic material is used as the solid phase 301. In addition, the method of immobilizing the primary antibody 302 on the substrate is preferably used when the fluorescence measurement system has the configuration in which the excitation light is emitted from one substrate side between the base substrate 102 and the cover substrate 106 to measure the fluorescence signal from the other substrate side.

In addition, the magnet is preferably arranged on a substrate side opposite to a side on which the irradiation of the light and the measurement of the reflected light are performed when the absorbance measurement system has the configuration in which the irradiation of the light and the measurement of the reflected light are performed on the same substrate side and the magnetic material is used as the solid phase 301. In addition, the method of immobilizing the primary antibody 302 on the substrate is preferably used when the absorbance measurement system has the configuration in which the light having the predetermined wavelength is emitted from one substrate side between the base substrate 102 and the cover substrate 106 to measure the transmitted light from the other substrate side.

### Modification 1

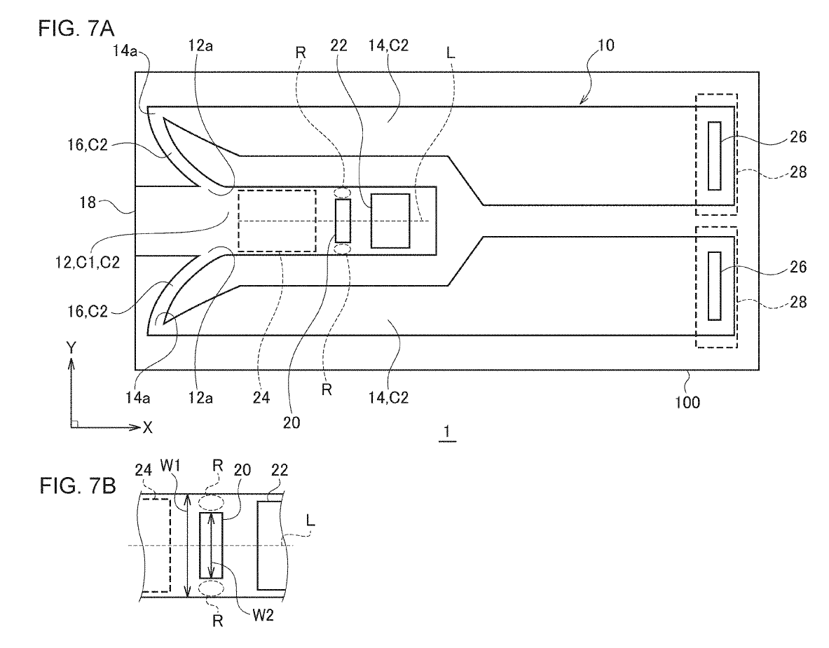

The sensor 1 according to the first embodiment described above can have Modification 1. Hereinafter, a configuration of the sensor 1 according to Modification 1 different from that of the first embodiment will be mainly described. The same configuration as that of the first embodiment will be denoted by the same reference numerals, and the description thereof will be simplified or omitted as appropriate. FIG. 7(A) is a plan view schematically illustrating an internal structure of the sensor 1 according to Modification 1 when viewed from the cover substrate 106 side. FIG. 7(B) is an enlarged view of the periphery of the first exhaust hole 20 in FIG. 7(A). For convenience of description, the first exhaust hole 20, the second liquid supply port 22, and the second exhaust hole 26 provided in the cover substrate 106 are also illustrated in FIG. 7(A).

In the sensor 1 according to the first embodiment, the second liquid supply port 22 also serves as the first exhaust hole 20. On the other hand, in the sensor 1 according to Modification 1, the second liquid supply port 22 is a separate body from the first exhaust hole 20 although communicating between the first chamber 10 and the outside of the substrate 100. The first exhaust hole 20 is arranged between the second liquid supply port 22 and the analyte trap 24 in the second flow channel C2 when viewed from the direction (Z-axis direction in FIG. 2) orthogonal to the main surface (for example, the second main surface 102b and the first main surface 106a) of the substrate 100.

In addition, the analyte trap 24 is arranged between the position 12a in the first part 12 to which the coupler 16 is connected and a position where the first exhaust hole 20 is provided in a direction in which the second liquid flows in the second flow channel C2. Therefore, the first exhaust hole 20 is arranged to be closer to the second liquid supply port 22 side than the position 12a of the first part 12 in the first flow channel C1 or the second flow channel C2. The first exhaust hole 20 may be provided on the base substrate 102 side or on the cover substrate 106 side.

In addition, the second flow channel C2 has a region R that does not overlap with the first exhaust hole 20 in a direction (Y-axis direction in FIG. 7(A)) orthogonal to a center line L of the second flow channel C2 at a position overlapping with the first exhaust hole 20 in a direction (X-axis direction in FIG. 7(A)) parallel to the center line L. In other words, a flow channel width (length W1 in a flow channel width direction) of a portion of the second flow channel C2 where the first exhaust hole 20 is positioned is larger than a length W2 of the first exhaust hole 20 in a direction parallel to the flow channel width direction. Alternatively, the length W2 of the first exhaust hole 20 in the direction orthogonal to the center line L of the second flow channel C2 is shorter than the length W1 in the corresponding direction of the portion of the second flow channel C2 where the first exhaust hole 20 is positioned. Alternatively, the length W2 of the first exhaust hole 20 in the direction orthogonal to the flow of the second liquid is shorter than the length W1 in the corresponding direction of the portion of the second flow channel C2 where the first exhaust hole 20 is positioned.

When the first exhaust hole 20 extends from one end side of the first part 12 to the other end side in the Y-axis direction of FIG. 7(A), it is difficult for the second liquid spotted to the second liquid supply port 22 to move to the analyte trap 24 side beyond the first exhaust hole 20. On the other hand, it is possible to prevent the movement of the second liquid from being inhibited by the first exhaust hole 20 by forming the region R that does not overlap with the first exhaust hole 20 in the second flow channel C2.

Incidentally, when the first exhaust hole 20 extends from one end side of the first part 12 to the other end side in the Y-axis direction of FIG. 7(A), it is necessary to close at least a part of the first exhaust hole 20 at the time of transferring the second liquid.

Next, a method of analyzing an analyte according to this embodiment will be described by exemplifying the sensor 1 according to Modification 1. The analyte analysis method according to the present embodiment includes the following steps A to C.

Step A: A first liquid F1 is supplied to the first liquid supply port 18 in a state where the second exhaust hole 26 is closed.

Step B: A second liquid F2 is supplied to the second liquid supply port 22 after Step A.

Step C: The second exhaust hole 26 is opened after the step A and before, after, or simultaneously with the step B.

In the step A, the first liquid F1 is transferred to the analyte trap 24 due to a capillary phenomenon and is further transferred to the first exhaust hole 20. In addition, the second liquid F2 is transferred from the second liquid supply port 22 to the analyte trap 24 due to the capillary phenomenon in the step B and the step C. Then, the second liquid F2 passes through the analyte trap 24, and the first liquid F1 is removed from the analyte trap 24. The second liquid F2 having passed through the analyte trap 24 is further transferred to the second exhaust hole 26.

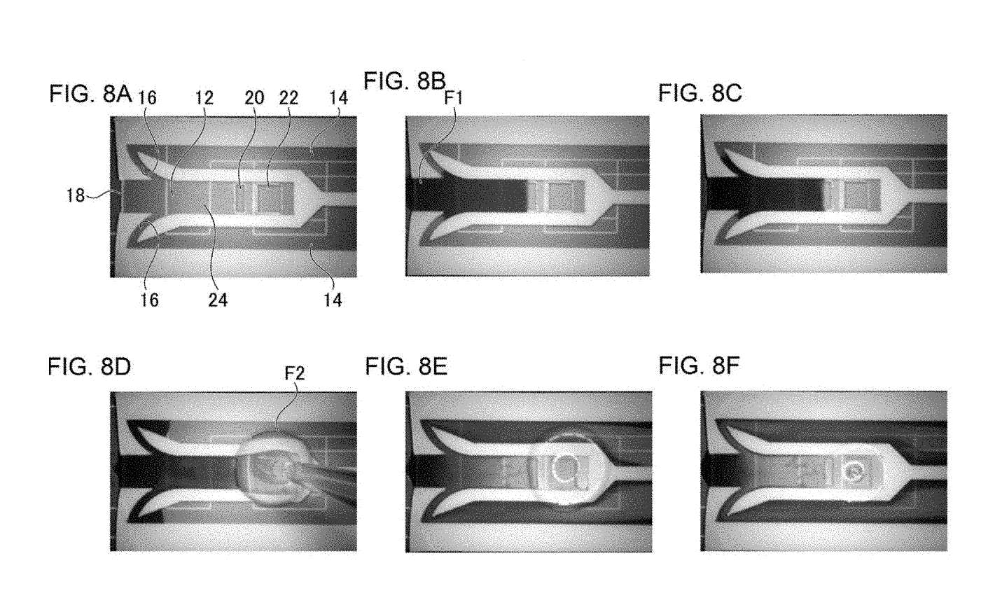

The inventor has actually confirmed the transfer of the first liquid and the second liquid using the sensor 1 according to Modification 1. FIGS. 8(A) to 8(F) are photographs illustrating a state where the first liquid and the second liquid are transferred in the sensor 1 according to Modification 1. Incidentally, the inventor has confirmed that the same result can be also obtained with the sensor 1 according to the first embodiment.

FIG. 8(A) is the photograph of the state of the sensor 1 before the first liquid F1 and the second liquid F2 are spotted to the first liquid supply port 18 and the second liquid supply port 22, respectively. Although the second exhaust hole 26 and the sealing member 28 are not illustrated, the second exhaust hole 26 is in the state of being closed by the sealing member 28. The first exhaust hole 20 is in the opened state.

FIG. 8(B) is the photograph of a state where the first liquid F1 is spotted to the first liquid supply port 18. When being spotted to the first liquid supply port 18, the first liquid F1 is drawn into the first part 12 due to the capillary phenomenon and is transferred to the first exhaust hole 20. Incidentally, the whole blood was used as the first liquid F1 in this experiment.

FIG. 8(C) is the photograph of a state where the second exhaust hole 26 is opened. When the second exhaust hole 26 is opened, the first liquid F1 is slightly transferred to the second exhaust hole 26 side. In this experiment, the first liquid F1 has moved to the inside of the coupler 16.

FIGS. 8(D) to 8(F) are the photographs of state changes over time after the second liquid F2 has been spotted to the second liquid supply port 22. Time has elapsed in the order of FIGS. 8(D), 8(E), and 8(F). Incidentally, a wash solution was used as the second liquid F2 in this experiment. When the second liquid F2 is spotted to the second liquid supply port 22, the first liquid supply port 18 is closed by the first liquid F1 as illustrated in FIG. 8(D). In addition, the second exhaust hole 26 is opened. Thus, the second liquid F2 spotted to the second liquid supply port 22 is drawn into the first part 12 due to the capillary phenomenon as illustrated in FIG. 8(E). As a result, the first liquid F1 existing in the analyte trap 24 is pushed out by the second liquid F2 and transferred to the second part 14.

Then, the second liquid F2 is further drawn into the first part 12 with the lapse of time as illustrated in FIG. 8(F). Accordingly, the first liquid F1 and the second liquid F2 are transferred to the second part 14. As a result, the first liquid F1 is almost completely removed from the analyte trap 24. In this experiment, it was confirmed that most of the first liquid F1 was transferred to the second part 14, and the first liquid F1 existing in the analyte trap 24 was almost completely replaced with the second liquid F2.

Therefore, it is possible to wash the composite 308 existing in the analyte trap 24 with the second liquid F2 if the composite 308 is formed by the antigen-antibody reaction among the solid-phase immobilized antibody 303, the antigen 304, and the labeled antibody 307. That is, it is possible to perform the B/F separation only by spotting of the first liquid F1 and the second liquid F2 and opening of the second exhaust hole 26 according to the sensor 1.

### Example 1

The inventor has actually analyzed an analyte using the sensor 1 and measured an obtained signal in order to confirm that an analyte can be analyzed and measured using the sensor 1. In the present example, TnT was used as the analyte. In addition, a sandwich immunoassay method using magnetic particles as a solid phase was used for the analysis of TnT. In addition, an electrochemiluminescence method was used for measurement of a signal obtained by the analysis.

### Structure of Sensor

The sensor 1 according to Modification 1 was used in the present example. This sensor 1 has the electrode pattern illustrated in FIG. 5 on the first main surface 102a of the base substrate 102. An electrode material was platinum. In addition, a magnet was fixed at a position corresponding to the analyte trap 24 on the second main surface 102b of the base substrate 102. Accordingly, magnetic particles contained in the first liquid F1 are captured by the analyte trap 24. The magnet is in the state of being coupled to the sensor 1 throughout the analysis and measurement of TnT.

### Preparation of First Liquid

First, the following reagent to be used for preparation of the first liquid was prepared.

### (a) TnT Solution (Antigen 304)

TnT (30C-CP3037, manufactured by Fitzgerald Industries International) was dissolved in plasma components to have each final concentration of TnT of 0 nM, 0.1 nM (1.0×10⁻¹⁰ M), 1.0 nM (1.0×10⁻⁹ M), and 10 nM (1.0×10⁻⁸ M). Blood cell components were added to each of four solutions having different TnT concentrations to obtain a TnT solution with a hematocrit value of 45%.

### (b) TnT Antibody-Labeled Magnetic Particle Solution (Solid-Phase Immobilized Antibody 303)

A first troponin antibody (10-T85A, manufactured by Fitzgerald Industries International) was dissolved in phosphate buffered saline (PBS) at pH 7.4 to prepare 1 ml of a first troponin antibody solution having a concentration of the first troponin antibody of 2 μM. In addition, NHS-Biotin (21425, manufactured by PIERCE) was dissolved in PBS to prepare a NHS-Biotin solution having a final concentration of NHS-Biotin of 20 mM. Here, NHS is N-hydroxysuccinimide.

Then, 2 μl of the NHS-Biotin solution was added to 1 ml of the first troponin antibody solution, and the resultant was mixed by inversion at room temperature for 30 minutes. Thereafter, 1 ml of blocking buffer (0.5 M of glycine (077-00735, manufactured by Wako Pure Chemical Industries, Ltd.), 0.5 M of NaCl (191-01665 manufactured by Wako Pure Chemical Industries, Ltd.), pH 8.3) was added. Then, the resultant was mixed by inversion at room temperature for 30 minutes to prepare a biotinylated antibody solution (the primary antibody 302).

Further, PBS was added to the biotinylated antibody solution to prepare a solution having a final concentration of the first troponin antibody of 0.15 μM. Then, avidin-labeled magnetic particles (manufactured by Merck Ltd., a particle diameter of 2.6 μm, a solid content of 0.1%, also referred to as streptavidin-immobilized magnetic particles) were subjected to buffer displacement in PBS. A solution of the avidin-labeled magnetic particles (solid phase 301) whose buffer was replaced in PBS and the biotinylated antibody solution were added at a volume ratio of 1:2 to obtain a TnT antibody-labeled magnetic particle solution.

### (c) Ruthenium Complex-Labeled Antibody Solution (Labeled Antibody 307)

A second troponin antibody solution (4T-19, manufactured by Hytest, Ltd.) was dissolved in PBS to prepare a second troponin antibody solution having a final concentration of the second troponin antibody of 0.1 mM. In addition, NHS and WSC (1-ethyl-3-(3-dimethylaminopropyl) carbodiimide) were dissolved in PBS to prepare an NHS solution and a WSC solution, respectively, having a final concentration of 10 mM.

Then, 100 μl of each of the NHS solution and the WSC solution was added to 1000 μl of the second troponin antibody solution, and the resultant was mixed by inversion for one hour at room temperature and subjected to activation processing. In addition, ruthenium (2,2′-bipyridyl)₂ (4-[3-(N-hydroxysuccinimidyl-carboxy)propyl]-4′-methyl-2,2′-bipyridine)) (hereinafter, referred to as a “ruthenium complex”) was dissolved in PBS to prepare a ruthenium complex solution having a concentration of the ruthenium complex of 50 mM.

The second troponin antibody solution that has been subjected to the activation treatment was added with 20 μl of the obtained ruthenium complex solution. Then, the resultant was mixed by inversion at room temperature for 30 minutes to prepare a ruthenylated antibody solution. The ruthenylated antibody solution was caused to pass through a desalting column to remove the ruthenium complex that has not bound to the second troponin antibody. In addition, buffer replacement with PBS was performed. The antibody solution thus obtained was adjusted to have the final concentration of the antibody of 0.15 μM, thereby obtaining a ruthenium complex-labeled antibody solution.

In a reaction vessel different from the sensor 1, 10 μl of the TnT antibody-labeled magnetic particle solution and 10 μl of the ruthenium complex-labeled antibody solution were added and mixed with 10 μl of each TnT solution having the final concentration of TnT of 0 nM (negative control), 0.1 nM, 1.0 nM, and 10 nM, thereby obtaining plural types of the first liquid F1 having different TnT concentrations. Each of the first liquids F1 is a reaction solution in which an antigen-antibody reaction of TnT and a biotin-avidin reaction have been substantially completed. Each of the first liquids F1 contains a reaction product and an unreacted substance.

### Preparation of Second Liquid

A wash/TPA solution was prepared as the second liquid F2. Specifically, TPA and TWEEN (registered trademark) 20 were added to 0.1 M of phosphate buffer (pH 6.0) so as to have a concentration of TPA of 0.1% and a concentration of the TWEEN (registered trademark) 20 of 1%, thereby obtaining the wash/TPA solution.

### Analysis and Measurement of Analyte

Then, TnT was analyzed and measured according to the following procedures (1) to (3).

(1) After the preparation of the first liquid, 6 μl of the first liquid F1 was promptly spotted to the first liquid supply port 18 of the sensor 1, and the resultant was left at room temperature for five minutes.

(2) After the lapse of five minutes, 40 μl of the wash/TPA solution as the second liquid F2 was spotted to the second liquid supply port 22 of the sensor 1. Further, a hole was formed in the sealing member 28 using a needle to make the opened state of the second exhaust hole 26.

(3) The working electrode 30 and the counter electrode 32 of the sensor 1 were connected to a power supply, and a voltage of 2.4 V was applied. An intensity of luminescence accompanying with the application of voltage was measured using Infinite 200 (manufactured by Tecan Group Ltd.).

An analyte was analyzed and measured three times for each TnT concentration. One sensor 1 was used for one time of analysis and measurement. Therefore, twelve sensors 1 in total were used.

### Experimental Result

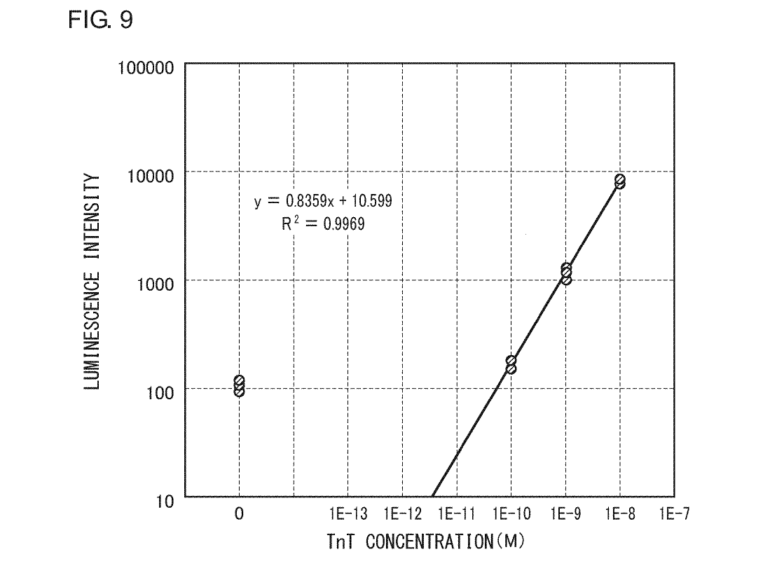

FIG. 9 is a graph illustrating measurement results of TnT in Example 1. As illustrated in FIG. 9, a difference in luminescence intensity was found between a sample with the negative control and a sample having the TnT concentration of 0.1 nM (1.0×10⁻¹⁰ M). In addition, it was confirmed that a calibration curve (y=0.8359x+10.599; R²=0.9969) prepared at three concentrations of 0.1 nM, 1.0 nM (1.0×10⁻⁹ M), and 10 nM (1.0×10⁻⁸ M) also depends on the TnT concentration. In addition, it was confirmed that there was little variation among samples having the same concentration and the reproducibility was high.

Based on this result, it was indicated that the sensor 1 according to Modification 1 can sufficiently perform the B/F separation and analyze and measure the analyte. Incidentally, it is possible to understand that the sensor 1 according to the first embodiment can also analyze and measure an analyte similarly to the sensor 1 according to Modification 1 based on the result of Example 1.

According to the sensor 1 according to the first embodiment or Modification 1 described above, it is possible to perform the B/F separation and analyze and measure the analyte with high accuracy only by spotting of the first liquid F1 to the first liquid supply port 18, spotting of the second liquid F2 to the second liquid supply port 22, and opening of the second exhaust hole 26. Therefore, it is possible to achieve both the simplification of the device used for the analyte measurement and the ease of the analyte measurement.

### Second Embodiment

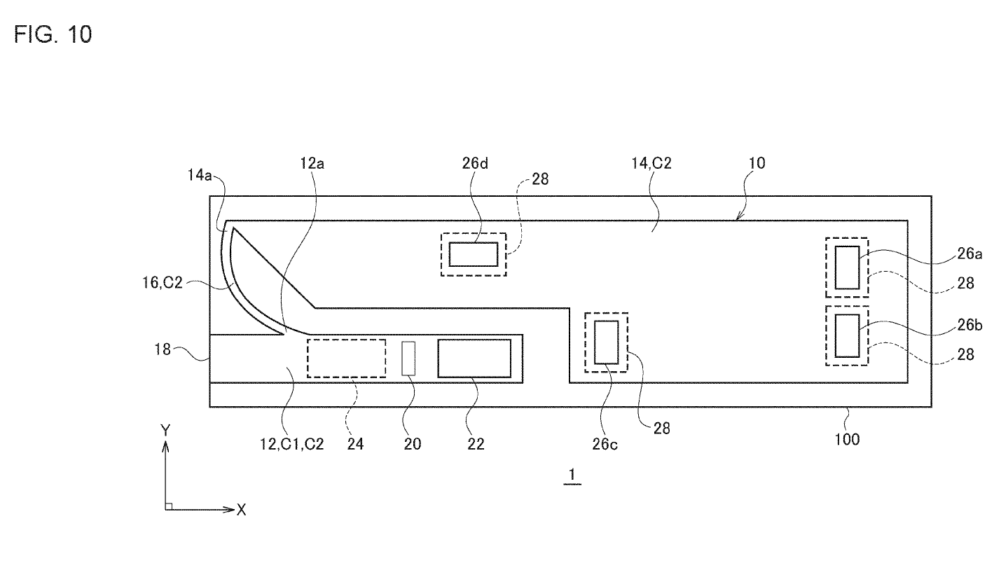

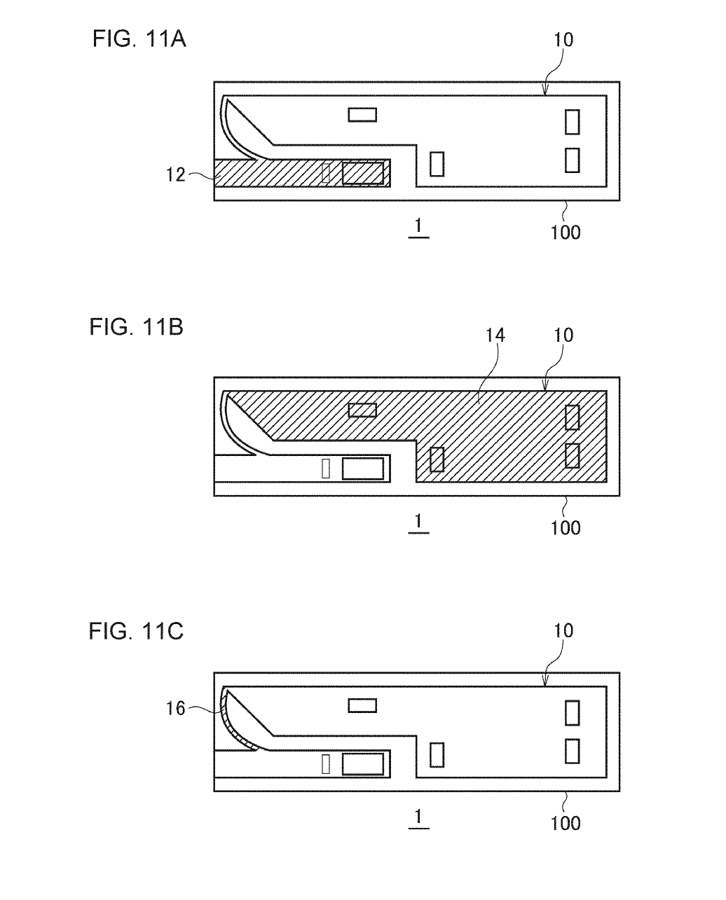

A sensor 1 according to the second embodiment has a configuration that is substantially common to that of the sensor 1 according to Modification 1 except that each number of the second part 14 and the coupler 16, a position and the number of the second exhaust holes 26 are different. Hereinafter, the sensor 1 according to the present embodiment will be described focusing on a configuration different from Modification 1. The same configuration as that of Modification 1 will be denoted by the same reference numerals, and the description thereof will be simplified or omitted as appropriate. FIGS. 10 and 11A, 11B and 11C are plan views schematically illustrating an internal structure of the sensor 1 according to the second embodiment when viewed from a cover substrate 106 side. For convenience of description, a first exhaust hole 20, a second liquid supply port 22, and a second exhaust hole 26 provided in the cover substrate 106 are also illustrated in FIGS. 10 and 11A, 11B and 11C.

The sensor 1 has a substrate 100 configured of a base substrate 102, a spacer member 104, and the cover substrate 106 (see FIG. 2). In the substrate 100, a first chamber 10 is provided. A lower surface of the first chamber 10 is defined by a first main surface 102a of the base substrate 102, a side surface is defined by a slit 104a of the spacer member 104, and an upper surface is defined by a second main surface 106b of the cover substrate 106.

The first chamber 10 includes a first part 12, a second part 14, and a coupler 16 connecting the first part 12 and the second part 14. The first part 12 is a hatched region in FIG. 11). The second part 14 is a hatched region in FIG. 11B. The coupler 16 is a hatched region in FIG. 11C. The sensor 1 according to the present embodiment has one first part 12, one second part 14, and one coupler 16.

The sensor 1 has a first liquid supply port 18, the first exhaust hole 20, and the second liquid supply port 22. Each of the first liquid supply port 18, the first exhaust hole 20, and the second liquid supply port 22 communicates between the first part 12 and the outside. The first liquid supply port 18 is defined by the first main surface 102a of the base substrate 102, the slit 104a of the spacer member 104, and the second main surface 106b of the cover substrate 106. In addition, the first exhaust hole 20 and the second liquid supply port 22 are provided as separately bodies in a region overlapping with the first part 12 of the cover substrate 106 in the normal direction of a main surface of the substrate 100. Incidentally, the second liquid supply port 22 may also serve as the first exhaust hole 20, which is similar to the first embodiment.

A space from the first liquid supply port 18 to the first exhaust hole 20 in the first part 12 is a space that causes a capillary phenomenon in a state where the first exhaust hole 20 is opened and forms a first flow channel C1. When a first liquid F1 is spotted to the first liquid supply port 18, the first liquid F1 is drawn into the first part 12 by the capillary phenomenon. The first liquid F1 drawn into the first part 12 is transferred to the first exhaust hole 20. An analyte trap 24 is arranged in the space between the first liquid supply port 18 and the first exhaust hole 20 in the first flow channel C1. Further, the analyte trap 24 is arranged in a space between a position 12a in the first part 12 to which the coupler 16 is connected and the first exhaust hole 20. A second liquid F2 is spotted to the second liquid supply port 22.

The sensor 1 has the second exhaust hole 26 communicating between the second part 14 and the outside. In the present embodiment, four second exhaust holes 26a, 26b, 26c, and 26d are provided as the second exhaust holes 26. Each of the second exhaust holes 26a to 26d are closed by the sealing member 28. The second exhaust hole 26a and the second exhaust hole 26b are arranged in the vicinity of an end portion on the opposite side of a position 14a in the second part 14 to which the coupler 16 is connected. Both the second exhaust hole 26c and the second exhaust hole 26d are arranged to be closer to the position 14a than the second exhaust holes 26a and 26b. The second exhaust hole 26d is arranged to be closer to the position 14a than the second exhaust hole 26c. The coupler 16 has one end portion connected to the first part 12, extends in a direction intersecting an extending direction of the first part 12, and has the other end portion connected to the second part 14.

A space formed of a portion from the second liquid supply port 22 to the position 12a in the first part 12, the coupler 16, and a portion from the position 14a to the second exhaust holes 26a to 26d in the second part 14 is a space that causes a capillary phenomenon in a state where the first liquid supply port 18 is closed and any of the second exhaust holes 26a to 26d is opened, and forms the second flow channel C2. When the second liquid F2 is spotted to the second liquid supply port 22, the second liquid F2 is drawn into the first part 12 due to the capillary phenomenon. The second liquid F2 drawn into the first part 12 passes through the coupler 16 due to the capillary phenomenon and is transferred to the opened exhaust hole side among the second exhaust holes 26a to 26d. The analyte trap 24 is arranged in a space between the second liquid supply port 22 and the coupler 16 in the second flow channel C2.

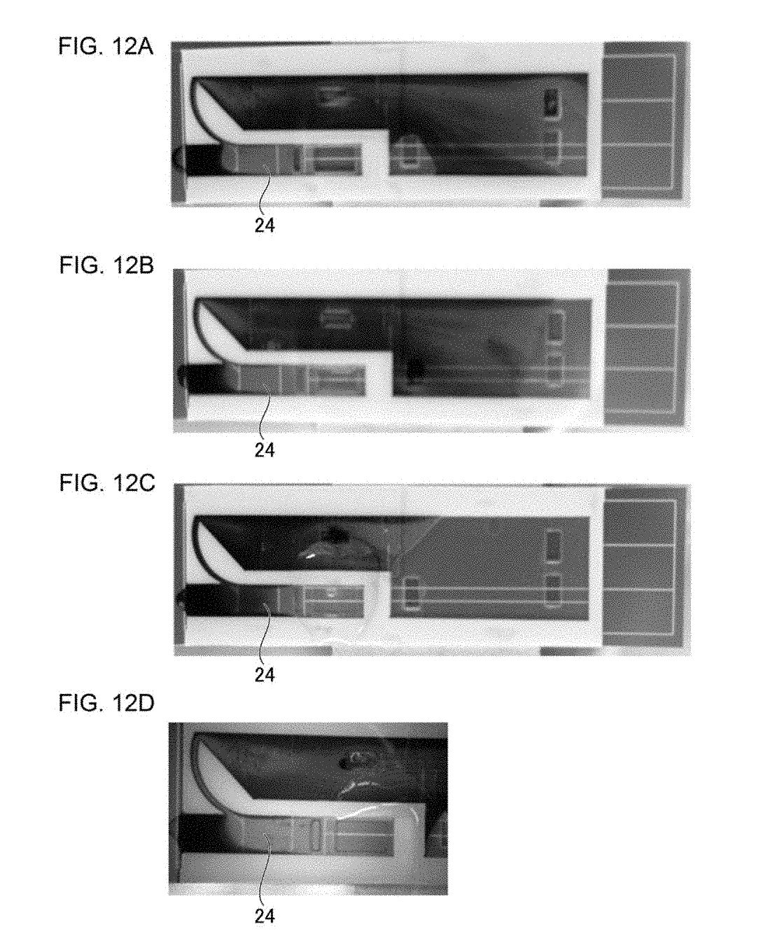

The inventor has actually confirmed the transfer of the first liquid F1 and the second liquid F2 using the sensor 1 according to the second embodiment. FIGS. 12A, 12B, 12C and 12D are photographs illustrating a state where the first liquid and the second liquid are transferred in the sensor 1 according to the second embodiment. FIGS. 12A, 12B, 12C and 12D illustrates the state after a wash solution as the second liquid F2 is spotted to the second liquid supply port 22 as any of the second exhaust holes 26a to 26d is opened after the first liquid F1 is spotted to the first liquid supply port 18. FIG. 12A illustrates a state when only the second exhaust hole 26b is opened. FIG. 12B illustrates a state when only the second exhaust hole 26c is opened. FIG. 12C illustrates a state when only the second exhaust hole 26d is opened. FIG. 12D illustrates a state where the second exhaust hole 26a and the second exhaust hole 26b are opened after the second exhaust hole 26d is opened (after the state in FIG. 12C).

It has been confirmed that the first liquid F1 and the second liquid F2 are transferred to the second part 14 so that the first liquid F1 existing in the analyte trap 24 is replaced with the second liquid F2 in a case where the second exhaust hole 26b or the second exhaust hole 26c is opened as illustrated in FIGS. 12A and 12B. Incidentally, it is possible to understand that the first liquid F1 existing in the analyte trap 24 can be replaced with the second liquid F2 even in a case where the second exhaust hole 26a is opened based on a result of the case where the second exhaust hole 26b is opened.

It has been confirmed that the first liquid F1 and the second liquid F2 are transferred to the second part 14 in a case where the second exhaust hole 26d is opened as illustrated in FIG. 12C. However, the first liquid F1 existing in the analyte trap 24 was not completely removed when the second exhaust hole 26d was opened.

The second exhaust hole 26d is arranged to be closer to the analyte trap 24 than the second exhaust holes 26a to 26c in the second flow channel C2. Therefore, a volume between the analyte trap 24 and the second exhaust hole 26d in the second flow channel C2 is smaller than a volume between the analyte trap 24 and the second exhaust holes 26a to 26c. It is considered that the first liquid F1 existing in the analyte trap 24 was not completely removed since this volume was smaller than a sum of the amount of the second liquid F2, necessary to remove the first liquid F1 existing in the analyte trap 24 from the analyte trap 24, and the amount of the first liquid F1 transferred to the second part 14 side by the second liquid F2.

This is supported by a result illustrated in FIG. 12D. That is, it has been confirmed that the first liquid F1 existing in the analyte trap 24 is replaced with the second liquid F2 when the second exhaust hole 26a and the second exhaust hole 26b are opened in the state where the second exhaust hole 26d is opened as illustrated in FIG. 12D. It is considered that such a result is obtained since the amount of the first liquid F1 and the second liquid F2 to be transferred to the second part 14 due to the capillary phenomenon is increased by the opening of the second exhaust hole 26a and the second exhaust hole 26b.

Based on the above experimental results, it has been confirmed that the first liquid F1 existing in the analyte trap 24 can be replaced with the second liquid F2 even when the only one second part 14 is provided. In addition, it has been confirmed that the position of the second exhaust hole 26 can be set as appropriate with a condition that the volume between the analyte trap 24 and the second exhaust hole 26 in the second flow channel C2 is equal to or larger than the volume of the sum of the amount of the second liquid F2 necessary to remove the first liquid F1 and the amount of the first liquid F1 to be removed. In addition, it has been confirmed that the plurality of second exhaust holes 26 may be provided. According to the present embodiment, it is possible to achieve the reduction in size of the sensor 1 since the number of the second parts 14 can be reduced.

### Third Embodiment

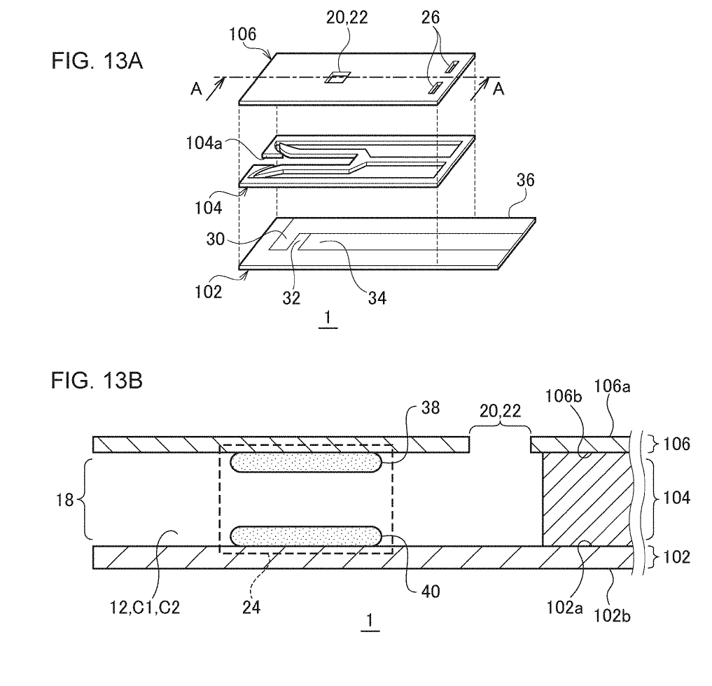

A sensor 1 according to the third embodiment has a configuration that is substantially common to the sensor 1 according to the first embodiment except that a first reagent layer 38 and a second reagent layer 40 are provided in a first part 12. Hereinafter, the sensor 1 according to the present embodiment will be described focusing on a configuration different from the first embodiment. The same configuration as that of the first embodiment will be denoted by the same reference numerals, and the description thereof will be simplified or omitted as appropriate. FIG. 13A is an exploded perspective view of the sensor 1 according to the third embodiment. FIG. 13B is an enlarged view of the periphery of an analyte trap 24 in a cross section taken along a line A-A of FIG. 13A. For example, the sensor 1 according to the present embodiment includes the electrode illustrated in FIG. 5 on a base substrate 102. Incidentally, the presence or absence of the electrode can be appropriately set in accordance with a measurement system to be adopted.

The sensor 1 includes the first reagent layer 38 and the second reagent layer 40 in a first flow channel C1. In the present embodiment, the first reagent layer 38 and the second reagent layer 40 are arranged in a space including the analyte trap 24 in the first flow channel C1. The first reagent layer 38 is fixed to a second main surface 106b of a cover substrate 106 and the second reagent layer 40 is fixed to a first main surface 102a of the base substrate 102. Incidentally, the first reagent layer 38 and the second reagent layer 40 may be arranged in a region other than the analyte trap 24 inside the first flow channel C1.

The first reagent layer 38 is, for example, a reagent layer containing a ruthenium complex-labeled antibody. The first reagent layer 38 is formed by, for example, dropping a predetermined amount of a ruthenium complex-labeled antibody solution onto the second main surface 106b of the cover substrate 106, and air-drying the solution. The second reagent layer 40 is, for example, a reagent layer containing a TnT antibody-labeled magnetic particle. The second reagent layer 40 is formed by, for example, dropping a predetermined amount of a TnT antibody-labeled magnetic particle solution onto the first main surface 102a of the base substrate 102, and air-drying the solution. The sensor 1 can be manufactured by forming the first reagent layer 38 and the second reagent layer 40 on the base substrate 102 and the cover substrate 106 before being bonded to each other, and then, bonding the base substrate 102, the spacer member 104, and the cover substrate 106 to each other.

Although the ruthenium complex-labeled antibody and the TnT antibody-labeled magnetic particle are contained in the separate reagent layers in the present embodiment, the invention is not limited to this configuration. For example, the sensor 1 may include only the first reagent layer 38 containing the ruthenium complex-labeled antibody and the TnT antibody-labeled magnetic particle. Alternatively, the sensor 1 may include only the second reagent layer 40 containing the ruthenium complex-labeled antibody and the TnT antibody-labeled magnetic particle. In addition, the first reagent layer 38 may contain the TnT antibody-labeled magnetic particle and the second reagent layer 40 may contain the ruthenium complex-labeled antibody. Both the first reagent layer 38 and the second reagent layer 40 may contain the TnT antibody-labeled magnetic particle and ruthenium s15 complex-labeled antibody.

In addition, magnetic particles are used as a solid phase 301 in the present embodiment, but TnT antibodies (primary antibodies 302) may be immobilized on a surface of a substrate forming the first flow channel C1, and a set of the immobilized TnT antibodies may be used as a reagent layer.

According to the sensor 1 of the present embodiment, it is possible to analyze and measure an analyte only by introducing an unprocessed specimen solution, such as blood, as a first liquid F1 into a first chamber 10 and then introducing a second liquid F2. Thus, it is possible to more easily analyze and measure the analyte.

### Example 2

The inventor has performed analysis of an analyte and measurement of an obtained signal using the sensor 1 according to the third embodiment. In the present example, TnT was used as the analyte. In addition, a sandwich immunoassay method using magnetic particles as a solid phase was used for the analysis of TnT. In addition, an electrochemiluminescence method was used for measurement of a signal obtained by the analysis.

### Preparation of Sensor

The sensor 1 according to the third embodiment was used in the present example. This sensor 1 has the electrode pattern illustrated in FIG. 5 on the first main surface 102a of the base substrate 102. An electrode material was platinum. In addition, a magnet was fixed at a position corresponding to the analyte trap 24 on the second main surface 102b of the base substrate 102. Accordingly, magnetic particles contained in the first liquid F1 are captured by the analyte trap 24. The magnet is in the state of being coupled to the sensor 1 throughout the analysis and measurement of TnT.

In addition, the first reagent layer 38 and the second reagent layer 40 were formed on the second main surface 106b of the cover substrate 106 and the first main surface 102a of the base substrate 102 according to the following procedure.

First, the following reagents to be used for the first reagent layer 38 and the second reagent layer 40 were prepared.

### (a) TnT Antibody-Labeled Magnetic Particle Solution (Solid-Phase Immobilized Antibody 303)

A first troponin antibody (10-T85A, manufactured by Fitzgerald Industries International) was dissolved in PBS having pH 7.4 to prepare 1 ml of a first troponin antibody solution having a concentration of the first troponin antibody of 2 μM. In addition, NHS-Biotin (21425, manufactured by PIERCE) was dissolved in PBS to prepare a NHS-Biotin solution having a final concentration of NHS-Biotin of 20 mM.

Then, 2 μl of the NHS-Biotin solution was added to 1 ml of the first troponin antibody solution, and the resultant was mixed by inversion at room temperature for 30 minutes. Thereafter, 1 ml of blocking buffer (0.5 M of glycine (077-00735, manufactured by Wako Pure Chemical Industries, Ltd.), 0.5 M of NaCl (191-01665 manufactured by Wako Pure Chemical Industries, Ltd.), pH 8.3) was added. Then, the resultant was mixed by inversion at room temperature for 30 minutes to prepare a biotinylated antibody solution (the primary antibody 302).

Further, PBS was added to the biotinylated antibody solution to prepare a solution having a final concentration of the first troponin antibody of 0.15 μM. Then, avidin-labeled magnetic particles (manufactured by Merck Ltd., a particle diameter of 2.6 μm, a solid content of 0.1%) were subjected to buffer displacement in PBS. An avidin-labeled magnetic particle solution (solid phase 301) whose buffer was replaced in PBS and the biotinylated antibody solution were added at a volume ratio of 1:2 to obtain a TnT antibody-labeled magnetic particle solution.

### (b) Ruthenium Complex-Labeled Antibody Solution (Labeled Antibody 307)

A second troponin antibody solution (4T-19, manufactured by Hytest, Ltd.) was dissolved in PBS to prepare a second troponin antibody solution having a final concentration of the second troponin antibody of 0.1 mM. In addition, NHS and WSC were dissolved in PBS to prepare an NHS solution and a WSC solution, respectively, having a final concentration of 10 mM.

Then, 100 μl of each of the NHS solution and the WSC solution was added to 1000 μl of the second troponin antibody solution, and the resultant was mixed by inversion for one hour at room temperature and subjected to activation processing. In addition, the ruthenium complex was dissolved in PBS to prepare a ruthenium complex solution having a concentration of the ruthenium complex of 50 mM. The second troponin antibody solution that has been subjected to the activation treatment was added with 20 μl of the obtained ruthenium complex solution. Then, the resultant was mixed by inversion at room temperature for 30 minutes to prepare a ruthenylated antibody solution.

The ruthenylated antibody solution was caused to pass through a desalting column to remove the ruthenium complex that has not bound to the second troponin antibody. In addition, buffer replacement with PBS was performed. The antibody solution thus obtained was adjusted to have the final concentration of the antibody of 0.15 μM, thereby obtaining a ruthenium complex-labeled antibody solution.

The TnT antibody-labeled magnetic particle solution and the ruthenium complex-labeled antibody solution thus obtained were mixed so as to have each antibody final concentration of 0.05 μM. Sucrose was added to this mixed solution so as to have the final concentration of 5%, and bovine serum albumin (BSA) was added so as to have the final concentration of 1%, thereby obtaining an antibody solution for formation of a reagent layer. Then, 3 μl of the obtained antibody solution was dropped onto a predetermined region of each of the base substrate 102 and the cover substrate 106. Thereafter, each of the substrates was placed inside a constant temperature bath at temperature of 50° C. and left for three minutes. The first reagent layer 38 and the second reagent layer 40 were formed through the above steps. Then, these substrates and the spacer member 104 were bonded to each other to manufacture the sensor 1.

### Preparation of First Liquid

A TnT (antigen) solution was prepared as the first liquid F1. Specifically, TnT (30C-CP3037, manufactured by Fitzgerald Industries International) was dissolved in standard serum to prepare five TnT solutions each of which has a different TnT concentration so as to have a TnT final concentration of 0 nM (negative control), 0.01 nM, 0.1 nM, 1.0 nM, and 10 nM.

### Preparation of Second Liquid

A wash/TPA solution was prepared as the second liquid F2. Specifically, TPA and TWEEN (registered trademark) 20 were added to 0.1 M of phosphate buffer (pH 6.0) so as to have a concentration of TPA of 0.1% and a concentration of the TWEEN (registered trademark) 20 of 1%, thereby obtaining the wash/TPA solution.

### Analysis and Measurement of Analyte

Then, TnT was analyzed and measured according to the following procedures (1) to (3).

(1) 6 μl of the TnT solution as the first liquid F1 was spotted to the first liquid supply port 18 of the sensor 1, and the resultant was left at room temperature for five minutes.

(2) After the lapse of five minutes, 40 μl of the wash/TPA solution as the second liquid F2 was spotted to the second liquid supply port 22 of the sensor 1. Further, a hole was formed in the sealing member 28 using a needle to make the opened state of the second exhaust hole 26.

(3) The working electrode 30 and the counter electrode 32 of the sensor 1 were connected to a power supply, and a voltage of 2.4 V was applied. An intensity of luminescence accompanying with the application of voltage was measured using Infinite 200 (manufactured by Tecan Group Ltd.).

An analyte was analyzed and measured once for each TnT concentration. One sensor 1 was used for one time of analysis and measurement. Therefore, five sensors 1 in total were used.

### Experimental Result

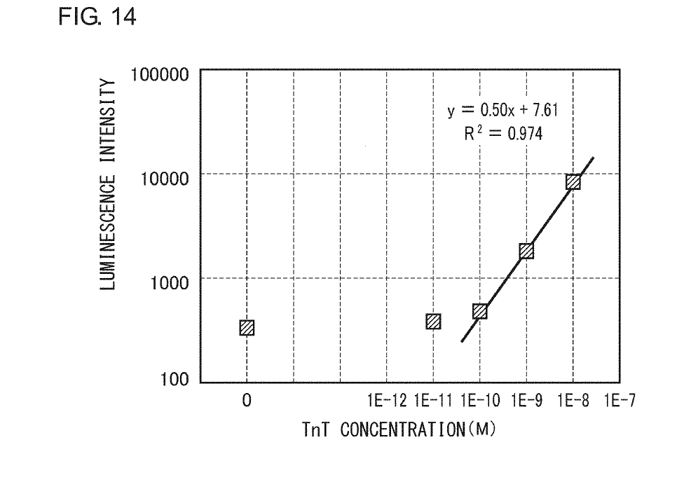

FIG. 14 is a graph illustrating measurement results of TnT in Example 2. As illustrated in FIG. 14, a difference in luminescence intensity was found between a sample with the negative control and a sample having the TnT concentration of 0.1 nM (1.0×10⁻¹⁰ M). In addition, it was confirmed that a calibration curve (y=0.50x+7.61; R²=0.974) prepared at three concentrations of 0.1 nM, 1.0 nM (1.0×10⁻⁹ M), and 10 nM (1.0×10⁻⁸ M) also depends on the TnT concentration.

Based on this result, it was indicated that the sensor 1 according to the third embodiment can sufficiently perform the B/F separation and analyze and measure the analyte. Therefore, according to the sensor 1 according to the third embodiment, it is possible to perform the B/F separation and analyze and measure the analyte with high accuracy only by spotting of the first liquid F1 to the first liquid supply port 18, spotting of the second liquid F2 to the second liquid supply port 22, and opening of the second exhaust hole 26. Therefore, it is possible to achieve both the simplification of the device used for the analyte measurement and the ease of the analyte measurement. In addition, the solid-phase immobilized antibody 303 and the labeled antibody 307 are provided in the sensor 1 in the present embodiment. As a result, it is possible to omit pretreatment of a specimen solution such as blood, and thus, it is possible to further simplify the preparation of the first liquid. Accordingly, it is possible to more easily analyze and measure the analyte.

### Fourth Embodiment

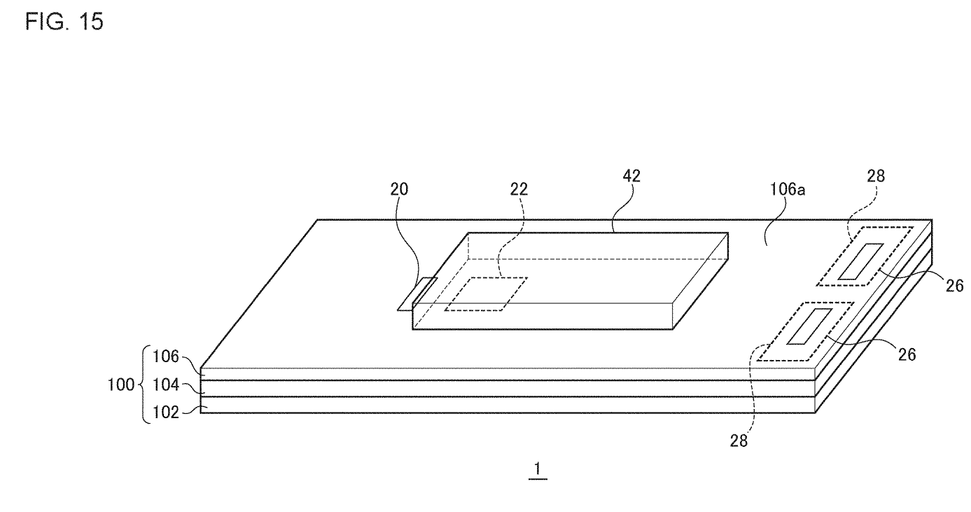

A sensor 1 according to the fourth embodiment has a configuration that is substantially common to the sensor 1 according to Modification 1 except that a container of a second liquid is provided. Hereinafter, the sensor 1 according to the present embodiment will be described focusing on a configuration different from Modification 1. The same configuration as that of Modification 1 will be denoted by the same reference numerals, and the description thereof will be simplified or omitted as appropriate. FIG. 15 is a perspective view illustrating a schematic structure of the sensor according to the fourth embodiment.

The sensor 1 according to the present embodiment has a substrate 100 configured of a base substrate 102, a spacer member 104, and a cover substrate 106. The cover substrate 106 is provided with a first exhaust hole 20, a second liquid supply port 22, and a second exhaust hole 26 that communicate between a first chamber 10 (see FIG. 7(A)) and the outside of the substrate 100. In addition, the sensor 1 is provided with a container 42 of a second liquid F2. The container 42 is, for example, a liquid holding bag, is arranged on an outer surface of the substrate 100, and is connected to the second liquid supply port 22. The container 42 is not particularly limited as long as being capable of holding a liquid, and examples thereof include an aluminum packaging material, a bag formed using a resin material such as polyethylene terephthalate (PET), polypropylene, and polyethylene, and the like.

The container 42 is fixed, for example, at a position, which covers the second liquid supply port 22, on a first main surface 106a of the cover substrate 106. In addition, the container 42 is fixed at a position that does not cover the first exhaust hole 20. Then, a needle is pierced through the container part 42 from the outside, for example, at a position where the container 42 and the second liquid supply port 22 overlap with each other in a stacking direction of the substrate 100 and the container 42, thereby forming a through-hole connecting the inside of the container 42 and the second liquid supply port 22 and a through-hole connecting the inside and the outside of the container 42. As a result, the second liquid F2 in the container 42 can be introduced into the first chamber 10 from the second liquid supply port 22 by a capillary force generated by opening of the second exhaust hole 26. Since the sensor 1 according to the present embodiment includes the container 42 of the second liquid F2, the analysis and measurement of the analyte can be further simplified.

Although the first exhaust hole 20 and the second liquid supply port 22 are separate bodies in the present embodiment, the first exhaust hole 20 and the second liquid supply port 22 may be integrated. That is, the container 42 may be provided in the sensor 1 according to the first embodiment. In this case, the container 42 is arranged so as to cover a part of the second liquid supply port 22. As a result, it is possible to secure a function of the first exhaust hole 20 provided in the second liquid supply port 22, that is, the function of generating a capillary force to transfer the first liquid F1 to the analyte trap 24.

### Fifth Embodiment

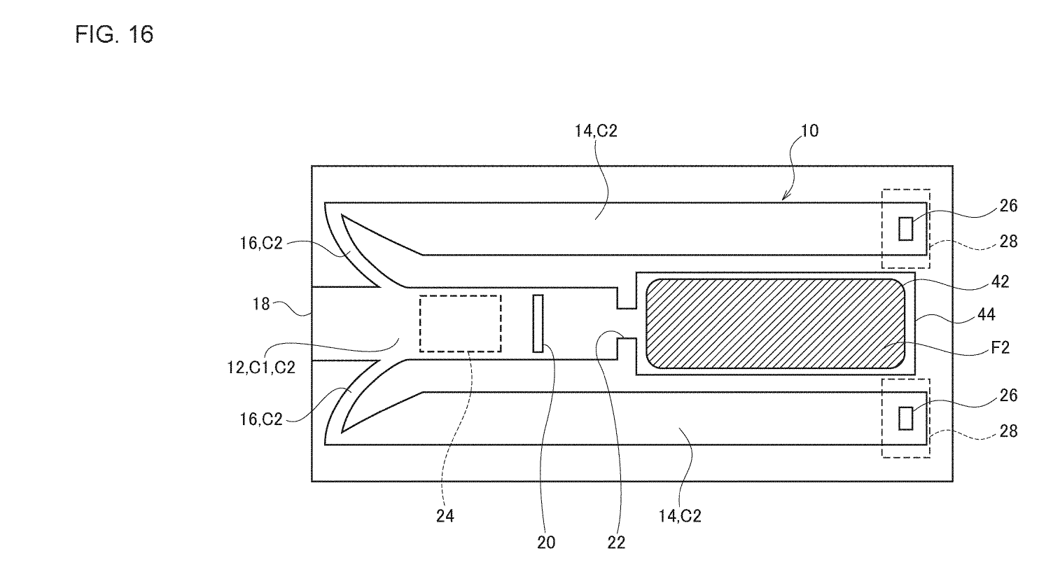

A sensor 1 according to the fifth embodiment has a configuration that is substantially common to the sensor 1 according to Modification 1 except that a second chamber 44 is provided and a first chamber 10 and a second chamber 44 communicate with each other. Hereinafter, the sensor 1 according to the present embodiment will be described focusing on a configuration different from Modification 1. The same configuration as that of Modification 1 will be denoted by the same reference numerals, and the description thereof will be simplified or omitted as appropriate. FIG. 16 is a plan view schematically illustrating an internal structure of the sensor 1 according to the fifth embodiment when viewed from a cover substrate 106 side. For convenience of description, a first exhaust hole 20 and a second exhaust hole 26 provided in the cover substrate 106 are also illustrated in FIG. 16.

The sensor 1 according to the present embodiment is also common to the sensor 1 according to the fourth embodiment in terms of holding a second liquid F2. However, the sensor 1 according to the present embodiment holds the second liquid F2 inside a substrate 100 while the sensor 1 according to the fourth embodiment holds the second liquid F2 outside the substrate 100.

Specifically, the sensor 1 according to the present embodiment includes the second chamber 44, which accommodates the second liquid F2, inside the substrate 100. The second chamber 44 is defined by a first main surface 102a of a base substrate 102, a slit 104a of a spacer member 104, and a second main surface 106b of the cover substrate 106. Then, a second liquid supply port 22 communicates between the first chamber 10 and the second chamber 44. The second liquid supply port 22 is defined by the first main surface 102a of the base substrate 102, the slit 104a of the spacer member 104, and the second main surface 106b of the cover substrate 106. Inside the second chamber 44, a container 42 having a volume to be fitted in the second chamber 44 is arranged, and the second liquid F2 is accommodated in the container 42.

In such a configuration, for example, a through-hole connecting the inside of the container 42 and the inside of the second chamber 44 is formed by piercing a needle through the container 42 from the outside of the substrate 100. As a result, the second liquid F2 in the container 42 can be introduced into the first chamber 10 from the second liquid supply port 22 by a capillary force generated by opening of the second exhaust hole 26. Since the sensor 1 according to the present embodiment includes the second chamber 44 that accommodates the second liquid F2, the analysis and measurement of the analyte can be further simplified.

### Sixth Embodiment

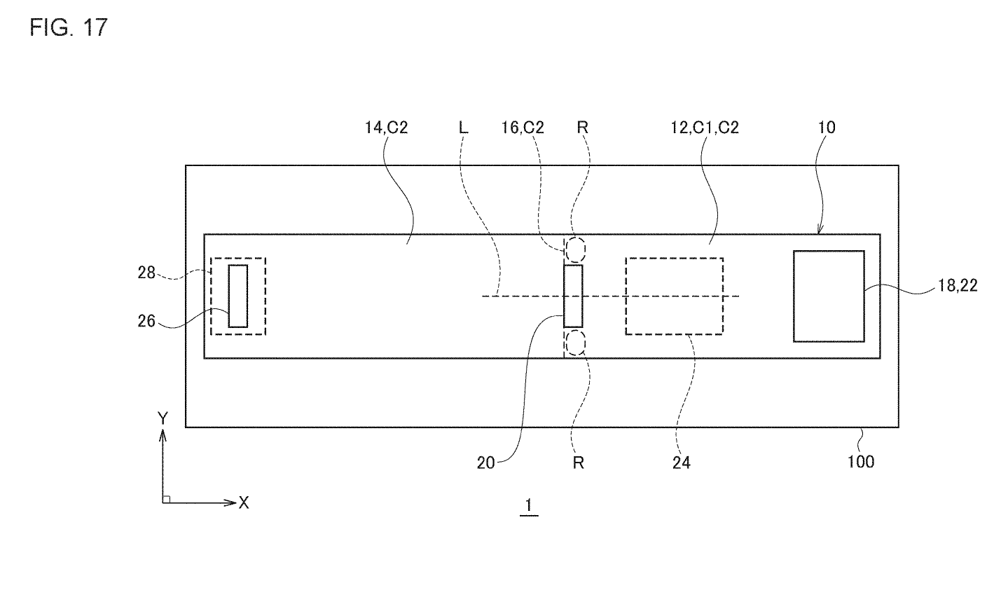

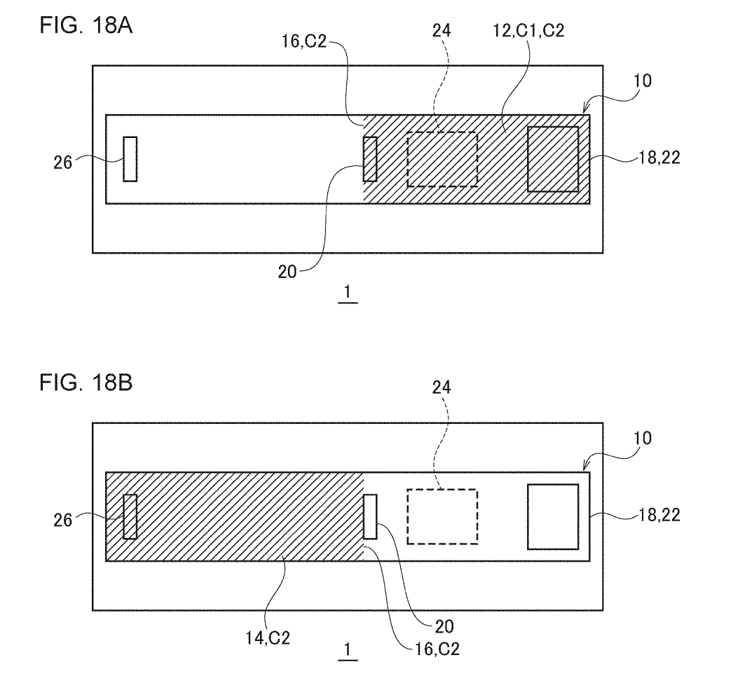

A sensor 1 according to the sixth embodiment has a configuration that is substantially common to the sensor 1 according to Modification 1 except that a second liquid supply port 22 also serves as a first liquid supply port 18 and that a second flow channel C2 has a substantially linear shape. Hereinafter, the sensor 1 according to the present embodiment will be described focusing on a configuration different from Modification 1. The same configuration as that of Modification 1 will be denoted by the same reference numerals, and the description thereof will be simplified or omitted as appropriate. FIGS. 17, 18A, and 18B are plan views schematically illustrating an internal structure of the sensor 1 according to the sixth embodiment when viewed from a cover substrate 106 side. For convenience of description, the first liquid supply port 18, a first exhaust hole 20, the second liquid supply port 22, and a second exhaust hole 26 provided in the cover substrate 106 are also illustrated in FIGS. 17, 18A, and 18B.

The sensor 1 according to the present embodiment has a first chamber 10 inside a substrate 100 formed by stacking a base substrate 102, a spacer member 104, and the cover substrate 106 (see FIG. 2). The first chamber 10 includes a first part 12, a second part 14, and a coupler 16 connecting the first part 12 and the second part 14. The first part 12 is a hatched region in FIG. 18A. The second part 14 is a hatched region in FIG. 18B. The coupler 16 is a boundary part between the first part 12 and the second part 14. In the sensor 1 of the present embodiment, the first part 12 and the second part 14 are adjacent to each other with the coupler 16 interposed therebetween and form one rectangular space. That is, the first part 12, the coupler 16, and the second part 14 are linearly arranged. In addition, the first part 12 and the second part 14 may extend in directions intersecting each other.

In addition, the sensor 1 includes the first liquid supply port 18, the first exhaust hole 20, the second liquid supply port 22, an analyte trap 24, and the second exhaust hole 26. The first liquid supply port 18 is a through-hole that communicates between the first chamber 10 and an outside of the substrate 100. More specifically, the first liquid supply port 18 communicates between the first part 12 of the first chamber 10 and the outside of the substrate 100. In the present embodiment, the first liquid supply port 18 is configured using a through-hole provided in the cover substrate 106.

The first exhaust hole 20 is a through-hole that communicates between the first chamber 10 and the outside of the substrate 100. More specifically, the first exhaust hole 20 communicates between the first part 12 and the outside of the substrate 100. In the present embodiment, the first exhaust hole 20 is configured using a through-hole provided in the cover substrate 106.

The second liquid supply port 22 is a through-hole that communicates between the first chamber 10 and the outside of the substrate 100. More specifically, the second liquid supply port 22 communicates between the first part 12 and the outside of the substrate 100. In addition, the second liquid supply port 22 also serves as the first liquid supply port 18. That is, the through-hole provided in the cover substrate 106 also serves as the first liquid supply port 18 and the second liquid supply port 22. The first part 12 is configured of a portion overlapping with the first liquid supply port 18 (the second liquid supply port 22), a portion between the first liquid supply port 18 and the first exhaust hole 20, and a portion overlapping with the first exhaust hole 20 when viewed from a direction (normal direction of a main surface) orthogonal to the main surface of the substrate 100.

The analyte trap 24 is a region which is positioned inside the first chamber 10 and by which an analyte in a first liquid F1 is captured. The analyte trap 24 is positioned in the first part 12.

The second exhaust hole 26 is a through-hole that communicates between the first chamber 10 and the outside of the substrate 100. More specifically, the second exhaust hole 26 communicates between the second part 14 of the first chamber 10 and the outside of the substrate 100. In the present embodiment, the second exhaust hole 26 is configured using a through-hole provided in the cover substrate 106. In the second exhaust hole 26, a sealing member 28 is provided. The second exhaust hole 26 can be switched from a closed state to an opened state by removing or perforating the sealing member 28. The second part 14 is configured of a portion between the first exhaust hole 20 and the second exhaust hole 26 and a portion overlapping with the second exhaust hole 26 when viewed from the direction orthogonal to the main surface of the substrate 100.

In the first chamber 10, a first flow channel C1 connecting the first liquid supply port 18, the analyte trap 24, and the first exhaust hole 20 is provided. More specifically, the first flow channel C1 is arranged in the first part 12. That is, a region of the first part 12 from the first liquid supply port 18 to the first exhaust hole 20 forms the first flow channel C1. The first liquid supply port 18 and the first exhaust hole 20 are arranged with the analyte trap 24 interposed therebetween in the first flow channel C1.

The second flow channel C2 connecting the second liquid supply port 22, the analyte trap 24, and the second exhaust hole 26 is provided inside the first chamber 10. More specifically, the second flow channel C2 is arranged across the first part 12, the coupler 16, and the second part 14. Therefore, the first flow channel C1 and the second flow channel C2 overlap with each other in a region between the first liquid supply port 18 and the first exhaust hole 20 in the first part 12. The second liquid supply port 22 and the second exhaust hole 26 are arranged with the analyte trap 24 interposed therebetween in the second flow channel C2.

In addition, the first exhaust hole 20 is arranged between the second exhaust hole 26 and the analyte trap 24 when viewed from the direction orthogonal to the main surface of the substrate 100. The second flow channel C2 has a region R that does not overlap with the first exhaust hole 20 in a direction (Y-axis direction in FIG. 17) orthogonal to a center line L of the second flow channel C2 at a position overlapping with the first exhaust hole 20 in a direction (X-axis direction in FIG. 17) parallel to the center line L. In other words, a flow channel width (length in the Y-axis direction of FIG. 17) of a portion of the second flow channel C2 where the first exhaust hole 20 is positioned is larger than a length of the first exhaust hole 20 in the flow channel width direction. Alternatively, a length of the first exhaust hole 20 in the direction orthogonal to the center line L of the second flow channel C2 is shorter than a length in the corresponding direction of the portion of the second flow channel C2 where the first exhaust hole 20 is positioned. Alternatively, the length of the first exhaust hole 20 in the direction orthogonal to the flow of a second liquid F2 is shorter than the length in the corresponding direction of the portion of the second flow channel C2 where the first exhaust hole 20 is positioned.

When the first exhaust hole 20 extends from one end side of the first part 12 to the other end side in the Y-axis direction of FIG. 17, it is difficult for the second liquid F2 spotted to the second liquid supply port 22 to move to the second exhaust hole 26 side beyond the first exhaust hole 20. On the other hand, it is possible to prevent the movement of the second liquid F2 from being inhibited by the first exhaust hole 20 by forming the region R that does not overlap with the first exhaust hole 20 in the second flow channel C2.

When the first liquid F1 is supplied to the first liquid supply port 18 in a state where the first liquid supply port 18 and the first exhaust hole 20 are opened and the second exhaust hole 26 is closed, the first liquid F1 is drawn into the first flow channel C1 from the first liquid supply port 18 along with discharge from the first exhaust hole 20. Then, the first liquid F1 reaches the analyte trap 24 and further moves to the first exhaust hole 20. That is, the first liquid F1 moves through the first flow channel C1 due to a capillary phenomenon, reaches the analyte trap 24, and is further drawn to the first exhaust hole 20.

When the second liquid F2 is supplied to the second liquid supply port 22 in a state where the second exhaust hole 26 is opened, the second liquid F2 is drawn into the second flow channel C2 from the second liquid supply port 22 along with discharge from the second exhaust hole 26. Then, the second liquid F2 passes through the analyte trap 24 and moves to the second exhaust hole 26 side. That is, the second liquid F2 moves through the second flow channel C2 due to a capillary phenomenon, passes through the analyte trap 24, and reaches the second part 14. The second liquid reaching the second part 14 is transferred to the second exhaust hole 26. As the second liquid F2 passes through the analyte trap 24, the first liquid F1 can be removed from the analyte trap 24. According to the present embodiment, it is possible to achieve further reduction in size of the sensor 1.

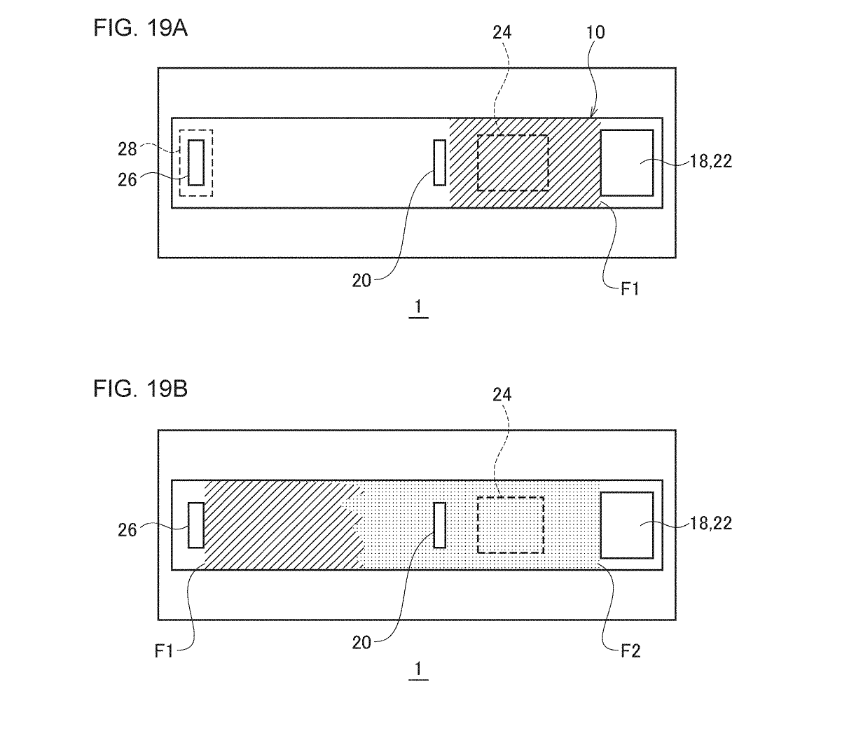

Next, a method of analyzing an analyte using the sensor 1 according to the sixth embodiment will be described. FIGS. 19A and 19B are plan views schematically illustrating a state where the first liquid F1 and the second liquid F2 are transferred in the sensor 1 according to the sixth embodiment.

The analyte analysis method using the sensor 1 according to the present embodiment includes the following steps A to C.

Step A: A first liquid F1 is supplied to the first liquid supply port 18 in a state where the second exhaust hole 26 is closed.

Step B: A second liquid F2 is supplied to the second liquid supply port 22 after Step A.

Step C: The second exhaust hole 26 is opened after the step A and before, after, or simultaneously with the step B.

By the step A, the first liquid F1 is drawn into the first chamber 10 via the first liquid supply port 18. Then, the first liquid F1 is transferred to the analyte trap 24 due to the capillary phenomenon and is further transferred to the first exhaust hole 20 as illustrated in FIG. 19A. In addition, the second liquid F2 is drawn into the first chamber 10 via the second liquid supply port 22 by the step B and the step C. Then, the second liquid F2 is transferred to the analyte trap 24 due to the capillary phenomenon and is further drawn into the second exhaust hole 26 side beyond the analyte trap 24 as illustrated in FIG. 19B. During this process, the first liquid F1 existing in the analyte trap 24 is pushed out by the second liquid F2 and removed from the analyte trap 24.

### Modification 2

The sensor 1 according to Modification 2 is common to the sensor 1 according to the sixth embodiment in terms of sharing the first liquid supply port 18 and the second liquid supply port 22. In addition, the sensor 1 according to Modification 2 is common to the sensor 1 according to the first embodiment except that a position of the first liquid supply port 18 and a position of the first exhaust hole 20 are interchanged with each other. Hereinafter, a configuration of the sensor 1 according to the present modification different from that of the first or sixth embodiment will be mainly described. The same configuration as that of the first or sixth embodiment will be denoted by the same reference numerals, and the description thereof will be simplified or omitted as appropriate.

In the sensor 1 according to the present modification, the first exhaust hole 20 in the sensor 1 illustrated in FIG. 3 functions as a supply port of the first liquid F1 (hereinafter referred to as a “first liquid supply port 18′”), and the first liquid supply port 18 functions as an exhaust hole (hereinafter referred to as a “first exhaust hole 20′”). Therefore, the second liquid supply port 22 also serves as the first liquid supply port 18′ in the sensor 1 according to the present modification. Even in the present modification, the first liquid supply port 18′ and the first exhaust hole 20′ are arranged with the analyte trap 24 sandwiched therebetween in the first flow channel C1. Then, the first liquid F1 spotted to the first liquid supply port 18′ is drawn into the first flow channel C1 along with discharge from the first exhaust hole 20′, reaches the analyte trap 24, and is further transferred to the first exhaust hole 20′ in a state where the second exhaust hole 26 is closed. A configuration of the second flow channel C2 is the same as that of the sensor 1 according to the first embodiment.

The analysis of an analyte using the sensor 1 according to the present modification includes the following steps A to C.

Step A: The first liquid F1 is spotted to the second liquid supply port 22, that is, the first liquid supply port 18′ in the state where the second exhaust hole 26 is closed.

Step B: A second liquid F2 is supplied to the second liquid supply port 22 after Step A.

Step C: The second exhaust hole 26 is opened after the step A and before, after, or simultaneously with the step B.

When the step A is performed, the first liquid F1 is transferred to the first exhaust hole 20′ via the analyte trap portion 24 due to the capillary phenomenon. In addition, when the step B and the step C are performed, the second liquid F2 is transferred from the second liquid supply port 22 to the second exhaust hole 26 side via the analyte trap portion 24 due to the capillary phenomenon. During this process, the first liquid F1 existing in the analyte trap 24 is pushed out by the second liquid F2 and moved to the second part 14. As a result, the B/F separation is implemented.

### Seventh Embodiment

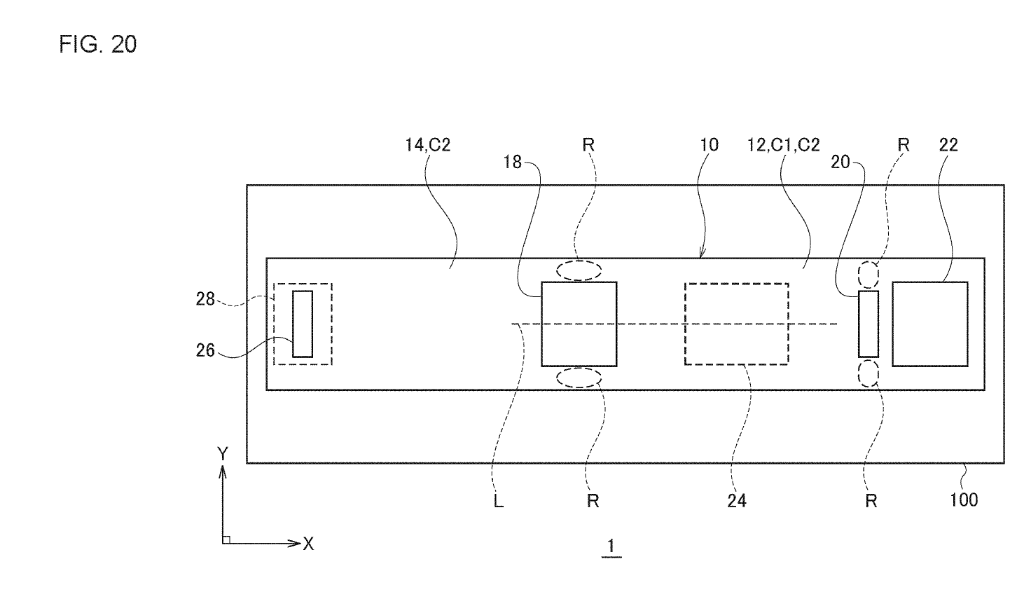

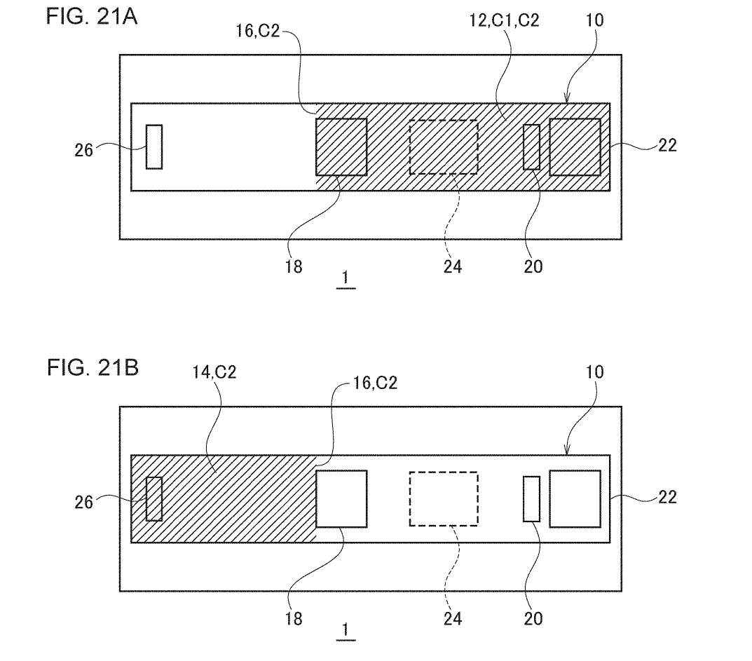

A sensor 1 according to the seventh embodiment has a configuration that is substantially common to the sensor 1 according to the sixth embodiment except that a first liquid supply port 18 and a second liquid supply port 22 are provided as separate bodies and that a first exhaust hole 20 is arranged to be closer to the second liquid supply port 22 than an analyte trap 24 and the first liquid supply port 18 is arranged to be closer to a second exhaust hole 26 than the analyte trap 24. Hereinafter, the sensor 1 according to the present embodiment will be described focusing on a configuration different from the sixth embodiment. The same configuration as that of the sixth embodiment will be denoted by the same reference numerals, and the description thereof will be simplified or omitted as appropriate. FIGS. 20, 21A, and 21B are plan views schematically illustrating an internal structure of the sensor 1 according to the seventh embodiment when viewed from a cover substrate 106 side. For convenience of description, the first liquid supply port 18, the first exhaust hole 20, the second liquid supply port 22, and the second exhaust hole 26 provided in the cover substrate 106 are also illustrated in FIGS. 20, 21A, and 21B.

The sensor 1 according to the present embodiment has a first chamber 10 inside a substrate 100 formed by stacking a base substrate 102, a spacer member 104, and the cover substrate 106 (see FIG. 2). The first chamber 10 includes a first part 12, a second part 14, and a coupler 16 connecting the first part 12 and the second part 14. The first part 12 is a hatched region in FIG. 21A. The second part 14 is a hatched region in FIG. 21B. The coupler 16 is a boundary part between the first part 12 and the second part 14. In the sensor 1 of the present embodiment, the first part 12 and the second part 14 are adjacent to each other with the coupler 16 interposed therebetween and form one rectangular space. That is, the first part 12, the coupler 16, and the second part 14 are linearly arranged. In addition, the first part 12 and the second part 14 may extend in directions intersecting each other.

In addition, the sensor 1 includes the first liquid supply port 18, the first exhaust hole 20, the second liquid supply port 22, an analyte trap 24, and the second exhaust hole 26. The first liquid supply port 18 is a through-hole that communicates between the first chamber 10 and an outside of the substrate 100. More specifically, the first liquid supply port 18 communicates between the first part 12 of the first chamber 10 and the outside of the substrate 100. In the present embodiment, the first liquid supply port 18 is configured using a through-hole provided in the cover substrate 106.

The first exhaust hole 20 is a through-hole that communicates between the first chamber 10 and the outside of the substrate 100. More specifically, the first exhaust hole 20 communicates between the first part 12 and the outside of the substrate 100. In the present embodiment, the first exhaust hole 20 is configured using a through-hole provided in the cover substrate 106.

The second liquid supply port 22 is a through-hole that communicates between the first chamber 10 and the outside of the substrate 100. More specifically, the second liquid supply port 22 communicates between the first part 12 and the outside of the substrate 100. In addition, the second liquid supply port 22 is the separate body from the first liquid supply port 18 in the present embodiment. The second liquid supply port 22 is also separated from the first exhaust hole 20. The second liquid supply port 22 is configured using a through-hole provided in the cover substrate 106. The first part 12 is configured of a portion overlapping with the first liquid supply port 18, a portion between the first liquid supply port 18 and the second liquid supply port 22, and a portion overlapping with the second liquid supply port 22 when viewed from a direction (normal direction of a main surface) orthogonal to the main surface of the substrate 100. Incidentally, the second liquid supply port 22 may also serve as the first exhaust hole 20, which is similar to Modification 1.

The analyte trap 24 is a region which is positioned inside the first chamber 10 and by which an analyte in a first liquid F1 is captured. The analyte trap 24 is positioned in the first part 12.

The second exhaust hole 26 is a through-hole that communicates between the first chamber 10 and the outside of the substrate 100. More specifically, the second exhaust hole 26 communicates between the second part 14 of the first chamber 10 and the outside of the substrate 100. In the present embodiment, the second exhaust hole 26 is configured using a through-hole provided in the cover substrate 106. In the second exhaust hole 26, a sealing member 28 is provided. The second exhaust hole 26 can be switched from a closed state to an opened state by removing or perforating the sealing member 28. The second part 14 is configured of a portion between the first liquid supply port 18 and the second exhaust hole 26 and a portion overlapping with the second exhaust hole 26 when viewed from the direction orthogonal to the main surface of the substrate 100.

In the first chamber 10, a first flow channel C1 connecting the first liquid supply port 18, the analyte trap 24, and the first exhaust hole 20 is provided. More specifically, the first flow channel C1 is arranged in the first part 12. That is, a region of the first part 12 from the first liquid supply port 18 to the first exhaust hole 20 forms the first flow channel C1. The first liquid supply port 18 and the first exhaust hole 20 are arranged with the analyte trap 24 interposed therebetween in the first flow channel C1.

The second flow channel C2 connecting the second liquid supply port 22, the analyte trap 24, and the second exhaust hole 26 is provided inside the first chamber 10. More specifically, the second flow channel C2 is arranged across the first part 12, the coupler 16, and the second part 14. Therefore, the first flow channel C1 and the second flow channel C2 overlap with each other in a region between the first liquid supply port 18 and the first exhaust hole 20 in the first part 12. The second liquid supply port 22 and the second exhaust hole 26 are arranged with the analyte trap 24 interposed therebetween in the second flow channel C2.

In addition, the second liquid supply port 22 and the second exhaust hole 26 are arranged such that the first liquid supply port 18, the analyte trap 24, and the first exhaust hole 20 are interposed therebetween when viewed from the direction orthogonal to the main surface of the substrate 100. In addition, with respect to the analyte trap 24, the first liquid supply port 18 and the second exhaust hole 26 are arranged on the same side, and the second liquid supply port 22 and the first exhaust hole 20 are arranged on the same side.

In addition, the second flow channel C2 has a region R that does not overlap with the first liquid supply port 18 in a direction (Y-axis direction in FIG. 20) orthogonal to a center line L of the second flow channel C2 at a position overlapping with the first liquid supply port 18 in a direction (X-axis direction in FIG. 20) parallel to the center line L when viewed from the direction orthogonal to the main surface of the substrate 100. In other words, a flow channel width of a portion of the second flow channel C2 where the first liquid supply port 18 is positioned is larger than a length of the first exhaust hole 20 in the flow channel width direction. Alternatively, a length of the first liquid supply port 18 in the direction orthogonal to the center line L of the second flow channel C2 is shorter than a length in the corresponding direction of the portion of the second flow channel C2 where the first liquid supply port 18 is positioned. Alternatively, a length of the first liquid supply port 18 in the direction orthogonal to the flow of the second liquid is shorter than a length in the corresponding direction of the portion of the second flow channel C2 where the first liquid supply port 18 is positioned.

In addition, the second flow channel C2 has a region R that does not overlap with the first exhaust hole 20 in the direction orthogonal to the center line L at the position overlapping with the first exhaust hole 20 in the direction parallel to the center line L when viewed from the direction orthogonal to the main surface of the substrate 100. In other words, a flow channel width of a portion of the second flow channel C2 where the first exhaust hole 20 is positioned is larger than a length of the first exhaust hole 20 in the flow channel width direction. Alternatively, a length of the first exhaust hole 20 in the direction orthogonal to the center line L of the second flow channel C2 is shorter than a length in the corresponding direction of the portion of the second flow channel C2 where the first exhaust hole 20 is positioned. Alternatively, the length of the first exhaust hole 20 in the direction orthogonal to the flow of a second liquid F2 is shorter than the length in the corresponding direction of the portion of the second flow channel C2 where the first exhaust hole 20 is positioned.

When the first liquid supply port 18 extends from one end side of the first part 12 to the other end side in the Y-axis direction of FIG. 20, it is difficult for the second liquid F2 spotted to the second liquid supply port 22 to move to the second exhaust hole 26 side beyond the first liquid supply port 18. Similarly, when the first exhaust hole 20 extends from one end side of the first part 12 to the other end side, it is difficult for the second liquid F2 spotted to the second liquid supply port 22 to move to the second exhaust hole 26 side beyond the first exhaust hole 20. On the other hand, it is possible to prevent the movement of the second liquid F2 from being inhibited by the first liquid supply port 18 and the first exhaust hole 20 by forming the region R that does not overlap with the first liquid supply port 18 and the region R that does not overlap with the first exhaust hole 20 in the second flow channel C2.

When the first liquid F1 is supplied to the first liquid supply port 18 in a state where the first liquid supply port 18 and the first exhaust hole 20 are opened and the second exhaust hole 26 is closed, the first liquid F1 is drawn into the first flow channel C1 from the first liquid supply port 18 along with discharge from the first exhaust hole 20. Then, the first liquid F1 reaches the analyte trap 24 and further moves to the first exhaust hole 20. That is, the first liquid F1 moves through the first flow channel C1 due to a capillary phenomenon, reaches the analyte trap 24, and is further drawn to the first exhaust hole 20.

When the second liquid F2 is supplied to the second liquid supply port 22 in a state where the second exhaust hole 26 is opened, the second liquid F2 is drawn into the second flow channel C2 from the second liquid supply port 22 along with discharge from the second exhaust hole 26. Then, the second liquid F2 passes through the analyte trap 24 and moves to the second exhaust hole 26 side. That is, the second liquid F2 moves through the second flow channel C2 due to a capillary phenomenon, passes through the analyte trap 24, and reaches the second part 14. As the second liquid F2 passes through the analyte trap 24, the first liquid F1 can be removed from the analyte trap 24. According to the present embodiment, it is possible to achieve further reduction in size of the sensor 1.

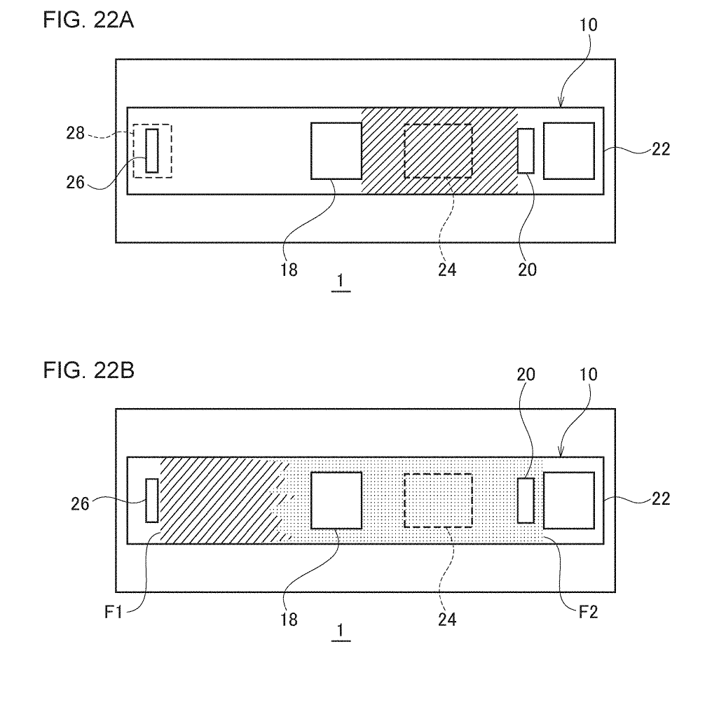

Next, a method of analyzing an analyte using the sensor 1 according to the seventh embodiment will be described. FIGS. 22A and 22B are plan views schematically illustrating a state where the first liquid F1 and the second liquid F2 are transferred in the sensor 1 according to the seventh embodiment.

The analyte analysis method using the sensor 1 according to the present embodiment includes the following steps A to C.

Step A: A first liquid F1 is supplied to the first liquid supply port 18 in a state where the second exhaust hole 26 is closed.

Step B: A second liquid F2 is supplied to the second liquid supply port 22 after Step A.

Step C: The second exhaust hole 26 is opened after the step A and before, after, or simultaneously with the step B.

By the step A, the first liquid F1 is drawn into the first chamber 10 via the first liquid supply port 18. Then, the first liquid F1 is transferred to the analyte trap 24 due to the capillary phenomenon and is further transferred to the first exhaust hole 20 as illustrated in FIG. 22A. In addition, the second liquid F2 is drawn into the first chamber 10 via the second liquid supply port 22 by the step B and the step C. Then, the second liquid F2 is transferred to the analyte trap 24 due to the capillary phenomenon and is further drawn into the second exhaust hole 26 side beyond the analyte trap 24 as illustrated in FIG. 22B. During this process, the first liquid F1 existing in the analyte trap 24 is pushed out by the second liquid F2 and removed from the analyte trap 24.

### Measurement Device

### Eighth Embodiment

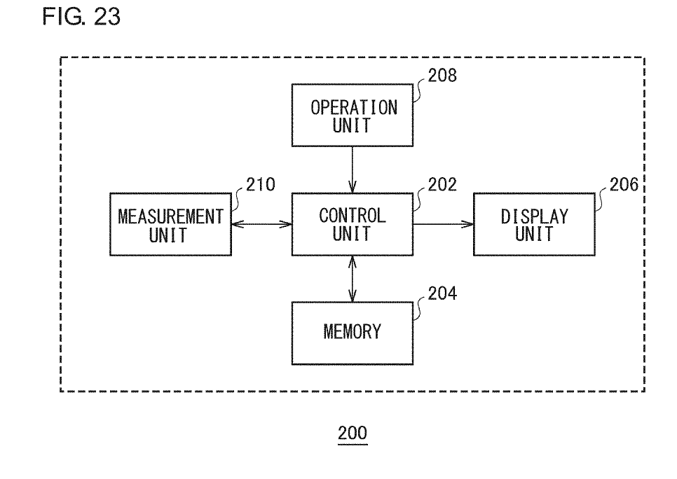

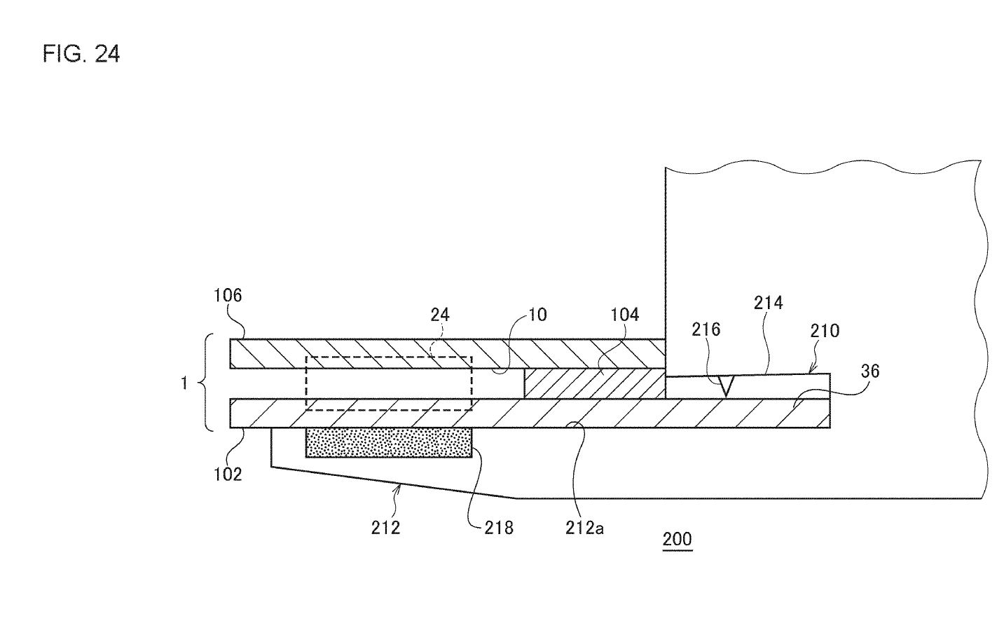

A measurement device using the sensor 1 according to each embodiment or each modification described above will be described. FIG. 23 is a block diagram schematically illustrating a functional configuration of a measurement device according to an eighth embodiment. In FIG. 23, each unit is drawn as a functional block. It is understood by those skilled in the art that such function blocks can be implemented in various forms using combinations of the hardware and the software. FIG. 24 is an enlarged cross-sectional view illustrating the periphery of a sensor support in the measurement device. For example, FIG. 24 illustrates the sensor support of the measurement device used in the sensor 1 for an electrochemical signal measurement system. In addition, FIG. 24 illustrates the sensor support of the measurement device used in a measurement system in which a magnetic material is used as a solid phase 301. In addition, FIG. 24 illustrates a cross section of the sensor 1 taken along the X-axis direction at a predetermined position in the Y-axis direction in FIG. 3.

A measurement device 200 according to the present embodiment is a device that measures an analyte by detecting a signal acquired by analyte analysis using the sensor 1. The measurement device 200 includes a control unit 202, a memory 204, a display unit 206, an operation unit 208, and a measurement unit 210 as illustrated in FIG. 23. In addition, the measurement device 200 includes a sensor support 212 as illustrated in FIG. 24.

### Control Unit

The control unit 202 executes various types of calculation, information processing, and the like, and controls each block of the measurement device 200. The control unit 202 is implemented by elements and circuits including a CPU of a computer as a hardware configuration, and implemented by a computer program or the like as a software configuration. The control unit 202 appropriately reads and executes a control program stored in the memory 204.

### Memory

The memory 204 is configured using, for example, a semiconductor memory, a magnetic recording medium, an optical recording medium, or the like. Various types of information including the control program of the measurement device 200 are stored in the memory 204.

### Display Unit

The display unit 206 is configured using, for example, a liquid crystal display or the like. The display unit 206 displays various types of information under the control of the control unit 202.

### Operation Unit

The operation unit 208 is configured to allow a user of the measurement device 200 to execute various input operations. Information input via the operation unit 208 is sent to the control unit 202 from the operation unit 208. Incidentally, the display unit 206 may also have the function of the operation unit 208. For example, the display unit 206 can also function as the operation unit 208 by including a touch panel including a sensor that detects contact of the user. The user can execute various operations of the measurement device 200 by, for example, touching a soft key displayed on the display unit 206. Incidentally, the configuration of the operation unit 208 incorporated in the display unit 206 is not limited to the touch panel type. In addition, the operation unit 208 may have a configuration in which a hard key is provided, a configuration in which a touch panel type and a hard key are combined, or the like.

### Measurement Unit

The measurement unit 210 detects and measures a signal generated by the sensor 1. The measurement unit 210 has various configurations necessary for measurement in accordance with a measurement system to be adopted in the sensor 1. Hereinafter, a configuration of the measurement unit 210 in accordance with each measurement system will be described.

### Electrochemical Signal Measurement System

When the measurement system of an analyte is an electrochemical signal measurement system, the measurement unit 210 includes a connector 214 as illustrated in FIG. 24. The connector 214 has an electrode 216. When the connection portion 36 of the sensor 1 is inserted into the connector 214, the electrode (see FIG. 5) provided in the sensor 1 is electrically connected to the electrode 216. The measurement unit 210 applies a predetermined voltage to the sensor 1 electrically connected via the electrode 216. As a result, the measurement unit 210 acquires a current value from the sensor 1.

Then, the measurement unit 210 transmits a signal indicating the obtained current value to the control unit 202. A conversion table in which a current value and an analyte concentration are associated with each other is stored in the memory 204. The control unit 202 measures the analyte concentration using the acquired current value and the conversion table. Then, the control unit 202 displays the obtained analyte concentration on the display unit 206, for example.

### Electrochemiluminescence Measurement System

When the analyte measurement system is an electrochemiluminescence measurement system, the measurement unit 210 includes the connector 214 and the electrode 216 similarly to the electrochemical signal measurement system. In addition, the measurement unit 210 includes a photodetector provided at a predetermined position. Examples of the photodetector include a photomultiplier tube (PMT), a charge coupled device (CCD), and the like. For example, the photodetector is arranged at a position facing the cover substrate 106 of the sensor 1 in a state where the sensor 1 is installed in the sensor support 212.

The measurement unit 210 applies a predetermined voltage to the sensor 1 electrically connected via the electrode 216. As a result, electrochemiluminescence occurs in the analyte trap 24 of the sensor 1. The measurement unit 210 detects this electrochemiluminescence by the photodetector. The measurement unit 210 transmits a signal indicating a light amount of detected electrochemiluminescence to the control unit 202. A conversion table in which a light amount and an analyte concentration are associated with each other is stored in the memory 204. The control unit 202 determines the analyte concentration using the acquired light amount and the conversion table. Then, the control unit 202 displays the obtained analyte concentration on the display unit 206, for example.

### Chemiluminescence/Bioluminescence Measurement System

When the analyte measurement system is a chemiluminescence/bioluminescence measurement system, the measurement unit 210 includes a photodetector similarly to the electrochemiluminescence measurement system. Since a configuration of the photodetector and a method of determining an analyte concentration by the control unit 202 are substantially similar to those of the electrochemiluminescence measurement system, the description thereof will be omitted.

### Fluorescence Measurement System

When the analyte measurement system is a fluorescence measurement system, the measurement unit 210 includes a light source and a photodetector provided at predetermined positions. The light source irradiates the sensor 1 with light having a predetermined wavelength to excite a fluorescent substance. Fluorescence occurs in the analyte trap 24 of the sensor 1 by the light emitted from the light source. The measurement unit 210 detects this fluorescence by the photodetector and transmits a signal indicating a light amount to the control unit 202. Since a configuration of the photodetector and a method of determining an analyte concentration by the control unit 202 are substantially similar to those of the electrochemiluminescence measurement system, the description thereof will be omitted.

### Absorbance Measurement System

When the analyte measurement system is an absorbance measurement system, the measurement unit 210 includes a light source and a light receiving element provided at predetermined positions. The light receiving element is configured using, for example, a photodiode or the like. The light source irradiates the sensor 1 with light having a predetermined wavelength. The light receiving element receives the light of the light source that has passed through the analyte trap 24 of the sensor 1. As a result, the measurement unit 210 acquires information on an absorbance.

The measurement unit 210 transmits a signal indicating the acquired information on the absorbance to the control unit 202. A conversion table in which an absorbance and an analyte concentration are associated with each other is stored in the memory 204. The control unit 202 determines the analyte concentration using the acquired absorbance and the conversion table. Then, the control unit 202 displays the obtained analyte concentration on the display unit 206, for example.

Next, a structure of the sensor support 212 will be described. The measurement device 200 has the sensor support 212 at a predetermined position as illustrated in FIG. 24. The sensor 1 is placed on the sensor support 212. For example, the sensor support 212 has a sensor mounting surface 212a. The sensor 1 is placed on the sensor mounting surface 212a such that the base substrate 102 is in contact with the sensor mounting surface 212a. The measurement unit 210 is adjacent to the sensor support 212. The sensor 1 is mounted on the sensor support 212, and the connection portion 36 is inserted into the connector 214.

In addition, the measurement device 200 includes a magnet 218 provided in the sensor support 212. The magnet 218 is arranged in the vicinity of the analyte trap 24 in a state where the sensor 1 is supported by the sensor support 212. For example, the magnet 218 is arranged at a position overlapping the analyte trap 24 when viewed from a direction in which the sensor support 212 and the sensor 1 are arrayed. When a magnetic material is used as the solid phase 301, that is, when the magnetic material is bonded to the analyte, the magnetic material is magnetized by the magnet 218 in the analyte trap 24. Accordingly, the analyte is captured in the analyte trap 24. As a result, it is possible to keep the analyte in the analyte trap 24 when the first liquid F1 existing in the analyte trap 24 is removed by the second liquid F2. Incidentally, a mechanism configured to perform perforating in the sealing member 28 or the container 42 or a mechanism configured to cause the second liquid F2 to be spotted to the second liquid supply port 22 may be provided in the sensor support 212 or the measurement unit 210.

Hereinafter, an analysis method that can be adopted by the above-described sensor 1 will be exemplified.

### (1) A Case where a Magnetic Material to which the Primary Antibody 302 is Bound is Used as the Solid-Phase Immobilized Antibody 303

In this case, the solid-phase immobilized antibody 303 is immobilized to the analyte trap 24 by the magnet 218. Incidentally, the solid-phase immobilized antibody 303 to be immobilized also include an antibody to which no analyte is bound.

### (1-1) A Case where the Entire Antigen-Antibody Reaction is Completed Before Introducing a Specimen Solution Containing an Analyte into the Sensor 1

First, the specimen solution, the solid-phase immobilized antibody 303, and the labeled antibody 307 are mixed to complete the antigen-antibody reaction. Subsequently, the obtained reaction solution is introduced into the sensor 1 as the first liquid F1. Then, the second liquid F2 is introduced into the sensor 1. As a result, the B/F separation and analyte analysis are performed.

### (1-2) A Case where Some Antigen-Antibody Reactions are Completed Before Introducing the Specimen Solution into the Sensor 1

First, the specimen solution and the solid-phase immobilized antibody 303 (or the labeled antibody 307) are mixed to complete a first antigen-antibody reaction. Subsequently, the obtained reaction solution is introduced into the sensor 1 as the first liquid F1. The labeled antibody 307 (or the solid-phase immobilized antibody 303) is previously provided in the sensor 1, and a second antigen-antibody reaction is completed in the sensor 1. Then, the second liquid F2 is introduced into the sensor 1. As a result, the B/F separation and analyte analysis are performed.

### (1-3) A Case where the Antigen-Antibody Reaction is not Carried Out Before Introducing the Specimen Solution into the Sensor 1

The specimen solution is introduced into the sensor 1 as the first liquid F1. The solid-phase immobilized antibody 303 and the labeled antibody 307 are provided in advance in the sensor 1, and the antigen-antibody reaction is completed in the sensor 1. Then, the second liquid F2 is introduced into the sensor 1. As a result, the B/F separation and analyte analysis are performed.

### (2) A Case where the Primary Antibody 302 is Immobilized on a Surface of a Substrate Defining the Analyte Trap 24

### (2-1) A Case where the Antigen-Antibody Reaction is Completed Before Introducing the Specimen Solution into the Sensor 1

First, the specimen solution and the labeled antibody 307 are mixed to complete a first antigen-antibody reaction. Subsequently, the obtained reaction solution is introduced into the sensor 1 as the first liquid F1. With the introduction of the first liquid F1, a second antigen-antibody reaction occurs between the analyte and the solid-phase immobilized antibody 303 immobilized in the sensor 1. Then, the second liquid F2 is introduced into the sensor 1. As a result, the B/F separation and analyte analysis are performed.

### (2-2) A Case where the Antigen-Antibody Reaction is not Carried Out Before Introducing the Specimen Solution into the Sensor 1

The specimen solution is introduced into the sensor 1 as the first liquid F1. The labeled antibody 307 is previously provided in the sensor 1, and further, the primary antibody 302 is immobilized to the substrate. Thus, the antigen-antibody reaction occurs among the analyte, the solid-phase immobilized antibody 303, and the labeled antibody 307 with the introduction of the first liquid F1. Then, the second liquid F2 is introduced into the sensor 1. As a result, the B/F separation and analyte analysis are performed.

### Sensor for Analyzing Analyte According to Another Aspect

Hereinafter, a sensor for analyte analysis according to another aspect will be described by exemplifying ninth to thirteenth embodiments.

### Ninth Embodiment

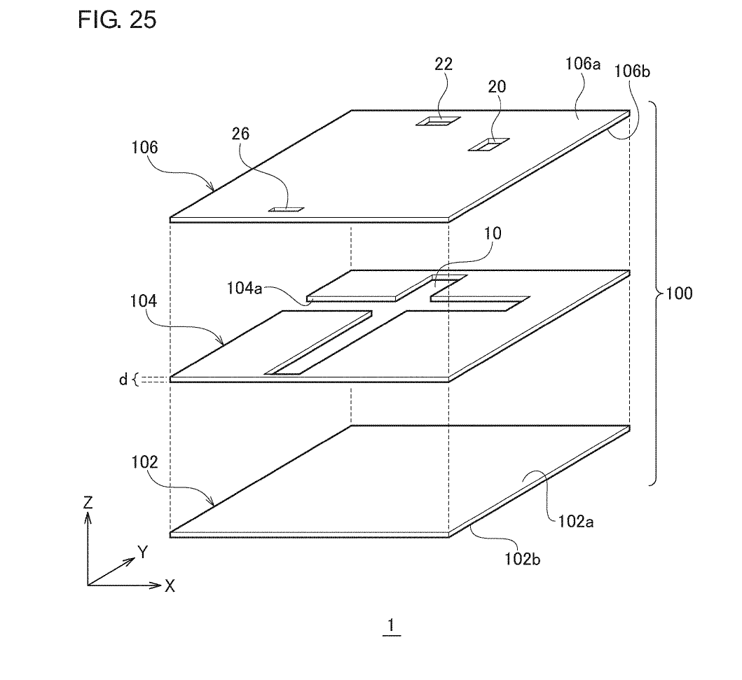

FIG. 25 is an exploded perspective view illustrating a schematic structure of a sensor according to the ninth embodiment. The sensor 1 according to the present embodiment is a sensor that analyzes an analyte and has a substrate 100. The substrate 100 includes a base substrate 102, a spacer member 104, and a cover substrate 106. The spacer member 104 is arranged on a surface of the base substrate 102. The cover substrate 106 is arranged on a surface of the spacer member 104 on a side opposite to the base substrate 102 side. The substrate 100 is formed by stacking the base substrate 102, the spacer member 104, and the cover substrate 106 in this order, and bonding these substrates to each other with an adhesive or the like.

Incidentally, the base substrate 102 and the spacer member 104 may be integrally formed, and the cover substrate 106 may be bonded to these base substrate 102 and spacer member 104, for example. In addition, the spacer member 104 and the cover substrate 106 may be integrally formed, and the base substrate 102 may be bonded to the spacer member 104 and the cover substrate 106. In addition, for example, a member formed using a resin material such as polyethylene terephthalate (PET), polystyrene, polycarbonate, and acrylic can be adopted as the base substrate 102, the spacer member 104, and the cover substrate 106. In addition, a substrate formed using glass may be adopted as the base substrate 102 and the cover substrate 106.

The respective substrates and member are attached to each other by, for example, an adhesive such as a hot-melt paste and a UV curable paste, or an adhesive tape. In this case, the spacer member 104 may be configured directly using the adhesive or the adhesive tape. That is, the spacer member 104 in the present application includes the adhesive or the adhesive tape. Alternatively, the respective substrates and member may be attached to each other by an ultrasonic welding method.

The base substrate 102 has a flat plate shape and has a first main surface 102a and a second main surface 102b opposite to the first main surface 102a. The spacer member 104 is stacked on the first main surface 102a.

The spacer member 104 is a planar member having a predetermined thickness d in a stacking direction (a Z-axis direction in FIG. 25) of the base substrate 102, the spacer member 104, and the cover substrate 106. In addition, the spacer member 104 has a slit 104a extending in a plane direction (XY directions in FIG. 25) of the spacer member 104. The slit 104a passes through the spacer member 104 in a direction of the thickness d. That is, the spacer member 104 has a shape in which a part of the flat plate is cut out by the slit 104a.

The cover substrate 106 has a flat plate shape and has a first main surface 106a and a second main surface 106b which is opposite to the first main surface 106a. The cover substrate 106 is stacked on the spacer member 104 such that the second main surface 106b faces the spacer member 104 side. The cover substrate 106 is provided with a first exhaust hole 20, a second liquid supply port 22, a second exhaust hole 26, and the like.

In the substrate 100, a first chamber 10 is provided. The first chamber 10 is formed by the first main surface 102a of the base substrate 102, the second main surface 106b of the cover substrate 106, and the slit 104a. That is, the first main surface 102a of the base substrate 102 defines a lower surface of the first chamber 10. A wall surface of the slit 104a of the spacer member 104 defines a side surface of the first chamber 10. The second main surface 106b of the cover substrate 106 defines an upper surface of the first chamber 10. Therefore, the first chamber 10 is a space defined by the base substrate 102, the spacer member 104, and the cover substrate 106.

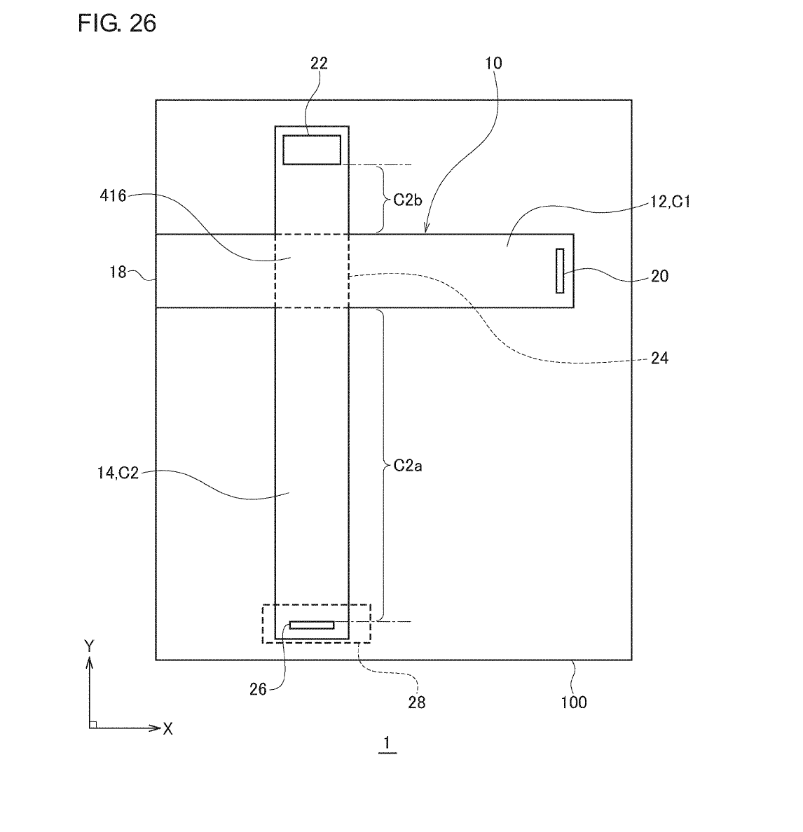

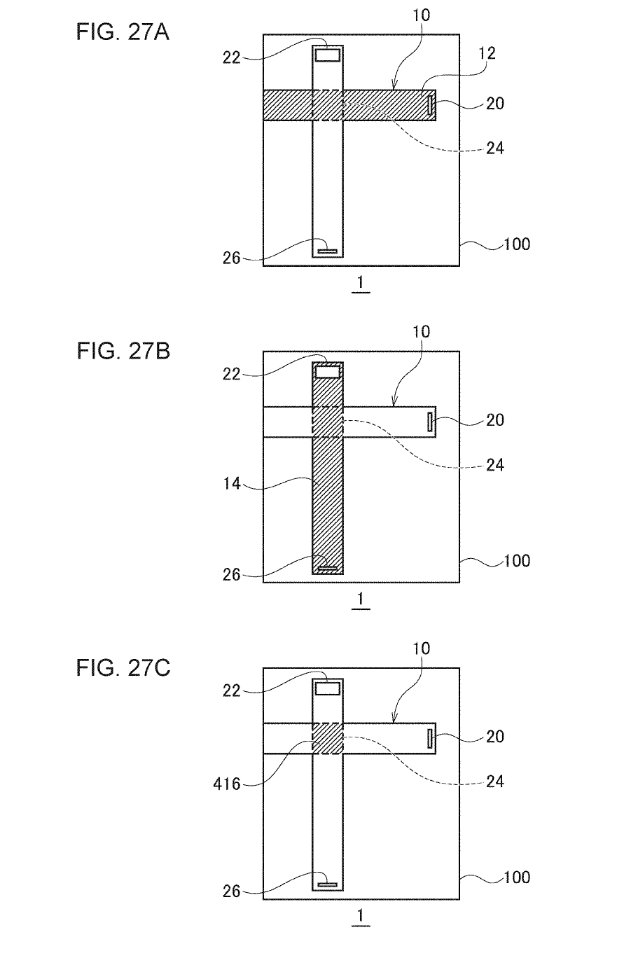

FIGS. 26 and 27A, 27B and 27C are plan views schematically illustrating an internal structure of the sensor 1 according to the ninth embodiment when viewed from a cover substrate 106 side. For convenience of description, a first exhaust hole 20, a second liquid supply port 22, and a second exhaust hole 26 provided in the cover substrate 106 are also illustrated in FIGS. 26 and 27A, 27B and 27C.

The first chamber 10 is arranged inside the substrate 100. The first chamber 10 includes a first part 12, a second part 14, and an intersection part 416 between the first part 12 and the second part 14. The first part 12 is a hatched region in FIG. 27A. The second part 14 is a hatched region in FIG. 27B. The intersection part 416 is a hatched region in FIG. 27C. The first part 12 and the second part 14 are linear and flat spaces, and cross each other to form the intersection part 416. Therefore, the intersection part 416 is included in both the first part 12 and the second part 14. In the present embodiment, the first part 12 and the second part 14 are orthogonal to each other.

In addition, the sensor 1 includes the first liquid supply port 18, the first exhaust hole 20, the second liquid supply port 22, an analyte trap 24, and the second exhaust hole 26. The first liquid supply port 18 is a through-hole that communicates between the first chamber 10 and an outside of the substrate 100. More specifically, the first liquid supply port 18 communicates between the first part 12 of the first chamber 10 and the outside of the substrate 100. In the present embodiment, the slit 104a extends to an outer surface (a side surface connecting the two main surfaces) of the spacer member 104, thereby forming the first liquid supply port 18. A first liquid containing an analyte is spotted to the first liquid supply port 18. As a result, the first liquid flows from the outside of the substrate 100 to the first chamber 10 via the first liquid supply port 18.

The first exhaust hole 20 is a through-hole that communicates between the first chamber 10 and the outside of the substrate 100. More specifically, the first exhaust hole 20 communicates between the first part 12 and the outside of the substrate 100. In the present embodiment, the first exhaust hole 20 is configured using the through-hole extending from the first main surface 106a to the second main surface 106b of the cover substrate 106. A gas in the first chamber 10 can flow to the outside of the substrate 100 via the first exhaust hole 20.

The second liquid supply port 22 is a through-hole that communicates between the first chamber 10 and the outside of the first chamber 10. More specifically, the second liquid supply port 22 communicates between the second part 14 and the outside of the first chamber 10. In the present embodiment, the second liquid supply port 22 communicates between the first chamber 10 and the outside of the substrate 100. In addition, the second liquid supply port 22 is configured using a through-hole extending from the first main surface 106a of the cover substrate 106 to the second main surface 106b. A second liquid containing a wash solution of the analyte trap 24 is spotted to the second liquid supply port 22. As a result, the second liquid flows from the outside of the first chamber 10 to the first chamber 10 via the second liquid supply port 22. Incidentally, the outside of the first chamber 10 to which the second liquid supply port 22 is connected may be another chamber provided inside the substrate 100. That is, the second liquid supply port 22 may communicate between the first chamber 10 and the other chamber in the substrate 100 (see the thirteenth embodiment to be described later).

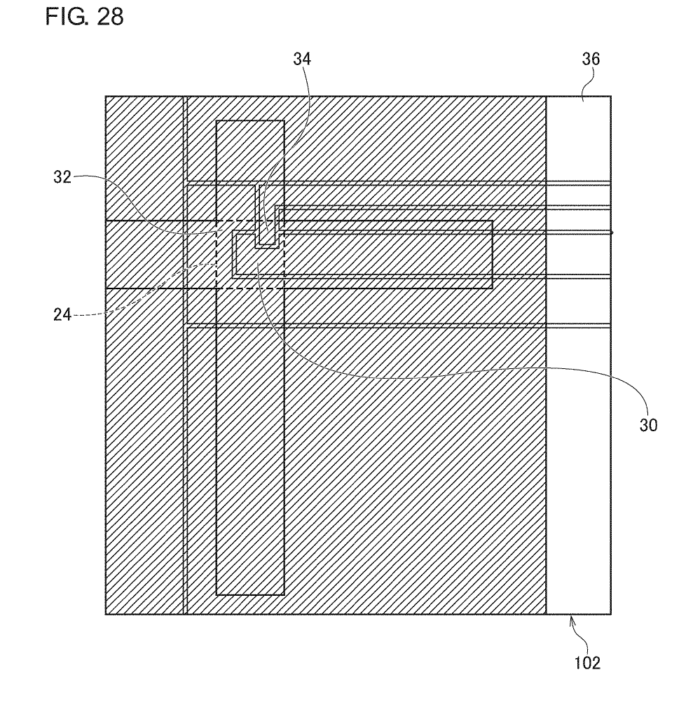

The analyte trap 24 is a region which is positioned inside the first chamber 10 and by which the analyte in the first liquid is captured. More specifically, the analyte trap 24 is arranged in the intersection part 416. Although the analyte trap 24 is arranged over the entire intersection part 416 in the present embodiment, the invention is not particularly limited to this configuration, and the analyte trap 24 may be arranged only in a part of the intersection part 416. For example, the analyte trap 24 corresponds to the solid phase 301, and the primary antibody 302 is immobilized to the surface of the base substrate 102 forming the analyte trap 24. Alternatively, when the solid phase 301 is made of a magnetic material, an analyte bound to the magnetic material is captured by the analyte trap 24 by a magnetic force of a magnet arranged in the vicinity of the analyte trap 24 (incidentally, a magnetic material to which the analyte is not bound is also captured by the analyte trap 24). In the analyte trap 24, the above-described signal of the label substance 305 is generated. That is, the analyte trap 24 corresponds to an analyte acquisition portion. When the label substance 305 is an electron mediator, at least a working electrode and a counter electrode are arranged in the analyte trap 24 (see FIG. 28).

The second exhaust hole 26 is a through-hole that communicates between the first chamber 10 and the outside of the substrate 100. More specifically, the second exhaust hole 26 communicates between the second part 14 of the first chamber 10 and the outside of the substrate 100. In the present embodiment, the second exhaust hole 26 is configured using the through-hole extending from the first main surface 106a to the second main surface 106b of the cover substrate 106. The second exhaust hole 26 can be switched from a closed state to an opened state. The gas in the first chamber 10 can flow to the outside of the substrate 100 via the second exhaust hole 26 in the opened state.

The sensor 1 includes a sealing member 28 that closes the second exhaust hole 26. The sealing member 28 is configured using, for example, an adhesive tape or the like and is provided on the first main surface 106a of the cover substrate 106 so as to cover the second exhaust hole 26. It is possible to switch the second exhaust hole 26 from the closed state to the opened state by removing this sealing member 28 or by making a hole in the sealing member 28.

Incidentally, the second exhaust hole 26 may be closed as the material forming the cover substrate 106 is present inside the second exhaust hole 26. That is, the second exhaust hole 26 may be closed by a part of the cover substrate 106. The part of the cover substrate 106 positioned inside the second exhaust hole 26 corresponds to the sealing member 28. This part may be integrated with another part around the second exhaust hole 26. In this case, the second exhaust hole 26 is opened, for example, as the user forms a hole in a formation region of the second exhaust hole 26 of the cover substrate 106 at the timing of generating a capillary force in the second flow channel C2. The cover substrate 106 is preferably subjected to processing to facilitate the formation of the second exhaust hole 26, such as making a thickness of a position where the second exhaust hole 26 is formed thinner than a thickness of the other region.

In the first chamber 10, a first flow channel C1 connecting the first liquid supply port 18, the analyte trap 24, and the first exhaust hole 20 is provided. More specifically, the first flow channel C1 is arranged in the first part 12. That is, a region of the first part 12 from the first liquid supply port 18 to the first exhaust hole 20 forms the first flow channel C1. The first flow channel C1 is a space extending from the first liquid supply port 18 to the first exhaust hole 20. Therefore, the first flow channel C1 has a linear shape. The first liquid supply port 18 and the first exhaust hole 20 are arranged with the analyte trap 24 interposed therebetween in the first flow channel C1.

When the first liquid is supplied to the first liquid supply port 18 in a state where the first liquid supply port 18 and the first exhaust hole 20 are opened and the second exhaust hole 26 is closed, the first liquid is drawn into the first flow channel C1 from the first liquid supply port 18 along with discharge from the first exhaust hole 20. Then, the first liquid reaches the analyte trap 24 and further moves to the first exhaust hole 20. That is, the first liquid supplied to the first liquid supply port 18 moves through the first flow channel C1 due to a capillary phenomenon, reaches the analyte trap 24, and is further drawn to the first exhaust hole 20.

The first liquid supply port 18 is arranged on a side surface of the substrate 100 in the present embodiment. Thus, the first liquid is spotted from the side of the sensor 1 (the X-axis direction in FIG. 26) to the first liquid supply port 18. Incidentally, the present invention is not particularly limited to this configuration. For example, the base substrate 102 or the cover substrate 106 may be provided with a through-hole communicating between the first chamber 10 and the outside of the substrate 100, and the first liquid supply port 18 may be configured using this through-hole. In this case, the first liquid is spotted to the first liquid supply port 18 from the lower side or the upper side the sensor 1 (the Z-axis direction in FIG. 25).

A size and a shape of the first liquid supply port 18 are not particularly limited as long as having an opening diameter that allows the first liquid spotted to the first liquid supply port 18 to move into the first chamber 10 by the capillary force. A size of the first flow channel C1 is not particularly limited as long as having a cross-sectional area that allows generation of the above-described capillary force. A size and a shape of the first exhaust hole 20 are not particularly limited as long as having an opening diameter that allows air to move from the first chamber 10 to the outside of the substrate 100.

The first liquid is not particularly limited as long as being a liquid containing at least an analyte. For example, the first liquid is a specimen solution collected from a human body such as blood or urine. In addition, the first liquid may be a liquid obtained by performing predetermined pretreatment to this specimen solution, or a mixture of this specimen solution and a reagent or the like.

The second flow channel C2 connecting the second liquid supply port 22, the analyte trap 24, and the second exhaust hole 26 is provided inside the first chamber 10. More specifically, the second flow channel C2 is arranged in the second part 14. That is, a region from the second liquid supply port 22 to the second exhaust hole 26 in the second part 14 forms the second flow channel C2. The second flow channel C2 is a space extending from the second liquid supply port 22 to the second exhaust hole 26. Therefore, the second flow channel C2 has a linear shape. The second liquid supply port 22 and the second exhaust hole 26 are arranged with the analyte trap 24 interposed therebetween in the second flow channel C2. The first flow channel C1 and the second flow channel C2 intersect each other at the analyte trap 24.

When the second liquid is supplied to the second liquid supply port 22 in a state where the first liquid supply port 18 is closed and the second exhaust hole 26 is opened, the second liquid is drawn into the second flow channel C2 from the second liquid supply port 22 along with discharge from the second exhaust hole 26. Then, the second liquid passes through the analyte trap 24 and moves to the second exhaust hole 26 side. That is, the second liquid moves through the second flow channel C2 due to a capillary phenomenon, passes through the analyte trap 24, and is transferred to the second exhaust hole 26. As the second liquid passes through the analyte trap 24, the first liquid can be removed from the analyte trap 24. The first liquid is drawn into a region C2a between the analyte trap 24 and the second exhaust hole 26 in the second flow channel C2 together with the second liquid.

The first liquid supply port 18 is closed as the first liquid is spotted to the first liquid supply port 18. That is, the first liquid supply port 18 is blocked by the first liquid. In addition, the second exhaust hole 26 is switched to the opened state by removing or perforating the sealing member 28 in the present embodiment. Thus, it is possible to easily control the timing at which the capillary force is generated in the second flow channel C2 to draw the second liquid into the second part 14.

In the present embodiment, the second liquid supply port 22 is arranged on the cover substrate 106. Thus, the second liquid is spotted to the second liquid supply port 22 from the upper side of the sensor 1 (the Z-axis direction in FIG. 25). Incidentally, the present invention is not particularly limited to this configuration. For example, the base substrate 102 may be provided with a through-hole communicating between the first chamber 10 and the outside of the substrate 100, and the second liquid supply port 22 may be configured using this through-hole. In this case, the second liquid is spotted to the second liquid supply port 22 from the lower side of the sensor 1 (the Z-axis direction in FIG. 25). In addition, the second liquid supply port 22 may be provided on a side surface of the substrate 100 similarly to the first liquid supply port 18. Similarly, the first exhaust hole 20 and the second exhaust hole 26 may be provided on the side surface of the substrate 100 or the base substrate 102.

A size and a shape of the second liquid supply port 22 are not particularly limited as long as having an opening diameter that allows the second liquid spotted to the second liquid supply port 22 to move into the first chamber 10 by the capillary force. A size of the second flow channel C2 is not particularly limited as long as having a cross-sectional area that allows generation of the above-described capillary force. A size and a shape of the second exhaust hole 26 are not particularly limited as long as having an opening diameter that allows air to move from the first chamber 10 to the outside of the substrate 100.

The second liquid is a liquid containing the wash solution to be used in B/F separation. Examples of the wash solution can include an aqueous solvent containing a surfactant. The surfactant used for the wash solution is preferably one that does not affect a reaction such as an antigen-antibody reaction. Examples of such a surfactant can include a non-ionic surfactant. Examples of the non-ionic surfactant include a TWEEN (registered trademark)-based surfactant (polyoxyethylene sorbitan fatty acid esters), and a TRITON (registered trademark)-based surfactant (polyoxyethylene p-t-octylphenyl ethers). In addition, the second liquid may contain a substrate to generate the signal corresponding to the label substance 305 as well as the wash solution. For example, when the analyte measurement system is a system that measures chemiluminescence or bioluminescence as a signal, the second liquid may contain a luminescent substrate, such as a luminol type and a dioxetane type, together with the wash solution. In addition, when the analyte measurement system is a system that measures electrochemiluminescence as a signal, the second liquid may contain an electron mediator, such as tripropylamine (TPA), together with the wash solution.

In addition, when the analyte measurement system is a system that measures an electrochemical signal, the second liquid may contain an electron mediator, such as potassium ferricyanide and a quinone compound, together with the wash solution. In addition, when the analyte measurement system is a system that measures an absorbance, that is, a dye as a signal, the second liquid may contain a chromogenic substrate together with the wash solution. Incidentally, the term “electron mediator” in the present specification refers to a substance that serves as a medium for exchange of electrons in an oxidation-reduction reaction. The electron mediator may be an oxidant or a reductant depending on a signal measurement system.

A volume of the region C2a between the analyte trap 24 and the second exhaust hole 26 in the second flow channel C2 is desirably larger than a sum of a volume of a region C2b between the second liquid supply port 22 and the analyte trap 24 in the second flow channel C2 and a volume of the analyte trap 24 in the second flow channel C2. It is necessary to replace the first liquid existing in the analyte trap 24 with the second liquid in the B/F separation. Thus, it is possible to reliably replace the first liquid existing in the analyte trap 24 with the second liquid by setting the volume of the region C2a and the total volume of the region C2b and the analyte trap 24 to have the above-described relationship.

At least a part of the wall surface inside the first chamber 10, for example, at least one of the first main surface 102a of the base substrate 102, the wall surface of the slit 104a of the spacer member 104, and the second main surface 106b of the cover substrate 106, and the first liquid supply port 18, the second liquid supply port 22, and the like may be subjected to predetermined hydrophilic treatment. It is possible to increase the capillary force generated in the first flow channel C1 or the second flow channel C2 by performing the hydrophilic treatment, and the liquid can be smoothly or reliably transferred due to the capillary phenomenon. Examples of the hydrophilic treatment can include application of a non-ionic, cationic, anionic, or amphoteric surfactant to the wall surface of the first chamber 10 or the liquid supply port, corona discharge treatment, and the like. Examples of the hydrophilic treatment can include formation of a fine uneven structure on the wall surface of the first chamber 10 or a surface of the liquid supply port, and the like (for example, see JP 2007-3361 A).

Next, a description will be given regarding the configuration of the sensor 1 in accordance with an analyte measurement method to be used, that is, a type of a signal to be measured. Each component of the sensor 1 according to the present embodiment can be changed in accordance with the analyte measurement method to be adopted.

### Electrochemical Signal Measurement System

When the analyte measurement system is a system that measures an electrochemical signal such as a current and a voltage, the label substance 305 in the labeled antibody 307 is, for example, an oxidoreductase. In this case, the sensor 1 acquires the electrochemical signal from an electron mediator through which electrons are exchanged by an oxidation-reduction reaction using the oxidoreductase. Alternatively, the sensor 1 acquires the electrochemical signal from hydrogen peroxide. The sensor 1 acquires these electrochemical signals using an electrode. In addition, the label substance 305 is, for example, an electron mediator such as ferrocene. In this case, for example, a current amplified by redox cycling is detected as the electrochemical signal, and the sensor 1 acquires this electrochemical signal by using the electrode.

FIG. 28 is a view schematically illustrating an example of an electrode pattern included in the sensor 1 according to the ninth embodiment. When the sensor 1 is used in the system that measures the electrochemical signal, at least the first main surface 102a of the base substrate 102 has an insulating property. Then, the sensor 1 has a working electrode 30 and a counter electrode 32 in a region corresponding to the analyte trap 24 of the base substrate 102. Not only the working electrode 30 and the counter electrode 32 but also a reference electrode 34 is provided in the present embodiment.

In addition, the sensor 1 has a connection portion 36 electrically connected to the measurement device. As the sensor 1 is electrically connected to the measurement device, the voltage or current for acquisition of the electrochemical signal is applied from the measurement device to the sensor 1. As this voltage or current is applied to the sensor 1, the electrochemical signal acquired by the sensor 1 through analyte analysis is measured by the measurement device. In FIG. 28, a hatched region is a region where the spacer member 104 and the cover substrate 106 are stacked. A region without hatching positioned at an end portion of the base substrate 102 is an exposed region of the base substrate 102. Each part of the working electrode 30, the counter electrode 32, and the reference electrode 34 is exposed in the exposed region. This exposed region forms the connection portion 36.

Examples of a material of the electrode include a metal material such as gold, platinum, and palladium, a carbon paste, or the like. The electrode can be formed on the base substrate 102, for example, as follows. That is, it is possible to form the electrode by forming a thin film having an electrode pattern shape on the first main surface 102a of the base substrate 102 by sputtering of a metal material. Alternatively, it is possible to form the electrode by performing laser cutting or the like to the thin film stacked on the first main surface 102a Alternatively, it is possible to form the electrode by printing a carbon paste having an electrode pattern shape on the first main surface 102a. Incidentally, the electrode and the connection portion 36 may be provided on the cover substrate 106.

### Electrochemiluminescence Measurement System

When the analyte measurement system is the system that measures electrochemiluminescence, the label substance 305 is an electrochemiluminescent body such as a ruthenium complex and an osmium complex. In this case, the sensor 1 acquires the luminescence of the electrochemiluminescent body, generated as a predetermined voltage is applied in the presence of an electron mediator such as TPA, as a signal. The sensor 1 has an electrode structure similar to that of the case of being used in the electrochemical signal measurement system. Incidentally, the electrochemiluminescence measurement system, the luminescence from the electrochemiluminescent body is measured on the cover substrate 106 side by the measurement device. Thus, at least a portion of the cover substrate 106 corresponding to the analyte trap 24 needs to have a light-transmitting property. Incidentally, the electrode and the connection portion 36 may be provided on the cover substrate 106, and luminescence may be measured on the base substrate 102 side. In this case, at least the portion of the base substrate 102 corresponding to the analyte trap 24 has a light-transmitting property.

### Chemiluminescence/Bioluminescence Measurement System

When the analyte measurement system is the system that measures chemiluminescence or bioluminescence, the label substance 305 is an enzyme such as peroxidase, alkaline phosphatase, and luciferase. In this case, as a chemiluminescent substrate is introduced into the analyte trap 24, a luminescent signal is generated from the chemiluminescent substrate by the label substance 305 existing in the analyte trap 24, that is, the enzyme. Incidentally, a chemiluminescent substance may be used as the label substance 305 instead of the enzyme, and the enzyme may be introduced into the analyte trap 24. In addition, a luminescent system that does not use enzymes, such as a luminescent system that generates a luminescent signal by a combination of a chemiluminescent substance and a luminescent catalytic substrate, may be adopted.

The luminescent signal acquired by the sensor 1 is measured on the base substrate 102 side or the cover substrate 106 side by the measurement device. Thus, a portion of the substrate on a side where the luminescence signal is measured corresponding to the analyte trap 24 needs to have a light-transmitting property. On the other hand, when a portion other than the portion corresponding to the analyte trap 24 also has the light-transmitting property, an unnecessary luminescent signal is measured so that the accuracy in measurement of the analyte is likely to decrease.

That is, the enzyme generates the luminescent signal immediately upon contact with the chemiluminescent/bioluminescent substrate. In addition, the chemiluminescent substance immediately generates the luminescent signal upon contact with the luminescent catalytic substrate. Thus, when the second liquid containing the luminescent substrate is supplied from the second liquid supply port 22 and drawn into the second exhaust hole 26 side after the first liquid reaches the analyte trap 24, the luminescent signal can also be generated from the luminescent substrate that has moved to be closer to the second exhaust hole 26 side than the analyte trap 24. When the whole substrate on the side where a photodetector of the measurement device is arranged has the light-transmitting property, a luminescent signal generated in a region other than the analyte trap 24 is also measured. Since such a luminescent signal becomes noise, there is a risk that the accuracy in measurement of the analyte may decrease.

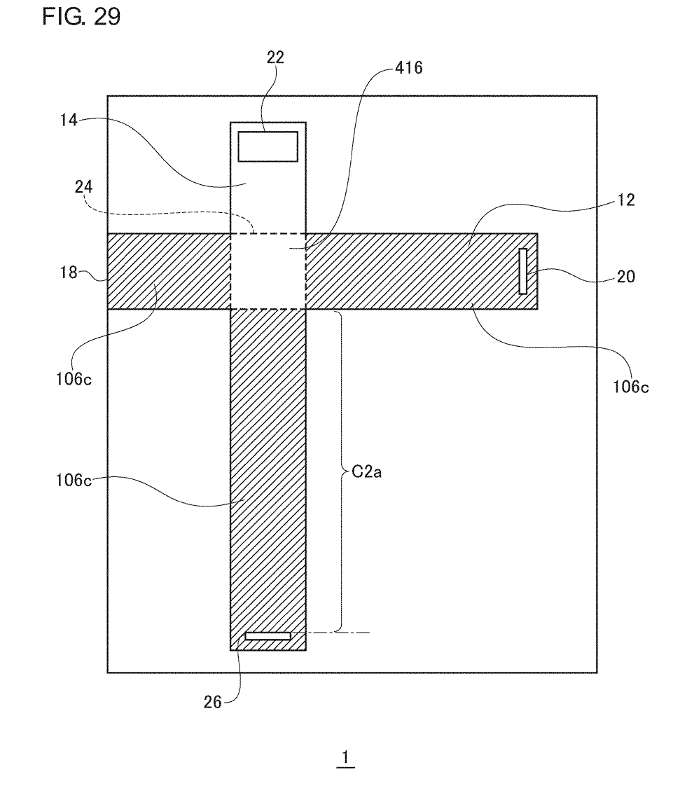

On the other hand, the substrate on the side where the photodetector is arranged has a light-shielding portion 106c in at least a partial region other than the portion corresponding to the analyte trap 24 in the sensor 1 according to the present embodiment. FIG. 29 is a view schematically illustrating an example of the light-shielding portion 106c of the sensor 1 according to the ninth embodiment. FIG. 29 illustrates the sensor 1 in the case where the cover substrate 106 includes the light-shielding portion 106c as an example.

As illustrated in FIG. 29, the sensor 1 has light-transmitting portions in a portion overlapping with the analyte trap 24 and a portion overlapping with a region closer to the second liquid supply port 22 side than the analyte trap 24 in the second part 14. In addition, the light-shielding portion 106c may be provided in each portion overlapping with a region in the first part 12 closer to the first liquid supply port 18 side than the analyte trap 24, a region in the first part 12 closer to the first exhaust hole 20 side than the analyte trap 24, and a region in the second part 14 closer to the second exhaust hole 26 side than the analyte trap 24. It is possible to suppress the luminescence signal serving as a noise source from being emitted to the outside of the substrate 100 by providing the light-shielding portion 106c. Incidentally, the sensor 1 is desirably provided with the light-shielding portion 106c at least at a portion overlapping with the region C2a. In addition, it is more preferable that the light-shielding portion 106c be provided in the entire portion except for the portion overlapping with the analyte trap 24.

### Fluorescence Measurement System

In the case where the analyte measurement system is a system that measures fluorescence, the label substance 305 is, for example, a fluorescent substance. In this case, the sensor 1 acquires fluorescence generated by irradiation of the fluorescent substance with excitation light as a signal. The label substance 305 is, for example, an enzyme such as alkaline phosphatase. In this case, for example, a fluorescent substrate such as 4-methylumbelliferyl phosphate is introduced, and the fluorescent substance obtained by a reaction of the fluorescent substrate and the enzyme is irradiated with excitation light, whereby fluorescence as a signal is generated.

Examples of the configuration of measuring the fluorescence signal can include a configuration in which excitation light is emitted from the base substrate 102 side to measure a fluorescence signal on the base substrate 102 side, and a configuration in which excitation light is emitted from the cover substrate 106 side to measure a fluorescent signal from the cover substrate 106 side. In this case, a substrate on a side where the irradiation of the excitation light and the measurement of the fluorescence signal are performed is configured such that at least a portion corresponding to the analyte trap 24 is made of a translucent material capable of transmitting the excitation light and the fluorescent signal therethrough.

In addition, a configuration in which excitation light is emitted from one substrate side between the base substrate 102 and the cover substrate 106 to measure a fluorescence signal on the other substrate side can be exemplified as another configuration of measuring the fluorescence signal. In this case, the substrate on the side where the excitation light is emitted is configured such that at least a portion corresponding to the analyte trap 24 is made of a translucent material capable of transmitting the excitation light. In this case, the substrate on the side where the fluorescence signal is measured is configured such that at least a portion corresponding to the analyte trap 24 is made of a translucent material capable of transmitting the fluorescent signal.

### Absorbance Measurement System

When the analyte measurement system is a system that measures an absorbance, the label substance 305 is, for example, an enzyme such as peroxidase or diaphorase. In this case, a chromogenic substrate is introduced into the analyte trap 24, and the chromogenic substrate and the enzyme react with each other so that a dye is generated from the chromogenic substrate. As the dye is irradiated with light having a predetermined wavelength, the absorbance as a signal is obtained.

Examples of a configuration of measuring the absorbance can include a configuration in which light having a predetermined wavelength is emitted from one substrate side between the base substrate 102 and the cover substrate 106 and the transmitted light is measured from the other substrate side. In this case, the base substrate 102 and the cover substrate 106 is configured such that at least a portion corresponding to the analyte trap 24 is made of a translucent material capable of transmitting the emitted light.

In addition, a configuration in which light having a predetermined wavelength is emitted from the base substrate 102 side and the reflected light is measured on the base substrate 102 side, and a configuration in which light having a predetermined wavelength is emitted from the cover substrate 106 side and the reflected light is measured on the cover substrate 106 side can be exemplified as other configurations of measuring the absorbance. In this case, the substrate on the side where the irradiation of light and the measurement of the reflected light are performed is configured such that at least a portion corresponding to the analyte trap 24 is made of a translucent material capable of transmitting the emitted light.

The sensor 1 according to the present embodiment can be used in any of a method of immobilizing the primary antibody 302 to a surface of any substrate corresponding to the analyte trap 24 and a method of immobilizing the primary antibody 302 to a magnetic material regardless of the analyte measurement system. That is, the substrate may be used as the solid phase 301, or the magnetic material may be used as the solid phase 301.

When the metal substrate is used as the solid phase 301, the primary antibody 302 can be immobilized to the surface of the substrate by, for example, a self-assembled monolayer (SAM). Other immobilizing methods include physical adsorption, chemical bonding, and the like. When the magnetic material is used as the solid phase 301, a magnet configured to capture the magnetic material in the analyte trap 24 is arranged in the vicinity of the analyte trap 24. The magnet is arranged, for example, on the second main surface 102b side of the base substrate 102 or on the first main surface 106a side of the cover substrate 106. Incidentally, the magnet may be provided in the sensor 1 or may be provided in the measurement device of the signal acquired by the sensor 1.

Incidentally, the magnet is preferably arranged on a substrate side opposite to a side on which the luminescence is measured when the magnetic material is used as the solid phase 301 in the electrochemiluminescence measurement system or the chemiluminescence/bioluminescence measurement system.

In addition, the magnet is preferably arranged on a substrate side opposite to a side on which the irradiation of the excitation light and the measurement of the fluorescence signal are performed when the fluorescence measurement system has the configuration in which the irradiation of the excitation light and the measurement of the fluorescence signal are performed on the same substrate side and the magnetic material is used as the solid phase 301. In addition, the method of immobilizing the primary antibody 302 on the substrate is preferably used when the fluorescence measurement system has the configuration in which the excitation light is emitted from one substrate side between the base substrate 102 and the cover substrate 106 to measure the fluorescence signal from the other substrate side.

In addition, the magnet is preferably arranged on a substrate side opposite to a side on which the irradiation of the light and the measurement of the reflected light are performed when the absorbance measurement system has the configuration in which the irradiation of the light and the measurement of the reflected light are performed on the same substrate side and the magnetic material is used as the solid phase 301. In addition, the method of immobilizing the primary antibody 302 on the substrate is preferably used when the absorbance measurement system has the configuration in which the light having the predetermined wavelength is emitted from one substrate side between the base substrate 102 and the cover substrate 106 to measure the transmitted light from the other substrate side.

Next, a method of analyzing an analyte according to the present embodiment will be described. The analyte analysis method according to the present embodiment includes the following steps AI to CI.

Step AI: A first liquid F1 is supplied to the first liquid supply port 18 in a state where the second exhaust hole 26 is closed.

Step BI: A second liquid F2 is supplied to the second liquid supply port 22 after Step AI.

Step CI: The second exhaust hole 26 is opened after the step AI and before, after, or simultaneously with the step BI.

In the step AI, the first liquid F1 is transferred to the analyte trap 24 due to a capillary phenomenon and is further transferred to the first exhaust hole 20. In addition, the second liquid F2 is transferred from the second liquid supply port 22 to the analyte trap 24 due to the capillary phenomenon in the step BI and the step CI. Then, the second liquid F2 passes through the analyte trap 24, and the first liquid F1 is removed from the analyte trap 24. The second liquid F2 having passed through the analyte trap 24 is further transferred to the second exhaust hole 26.

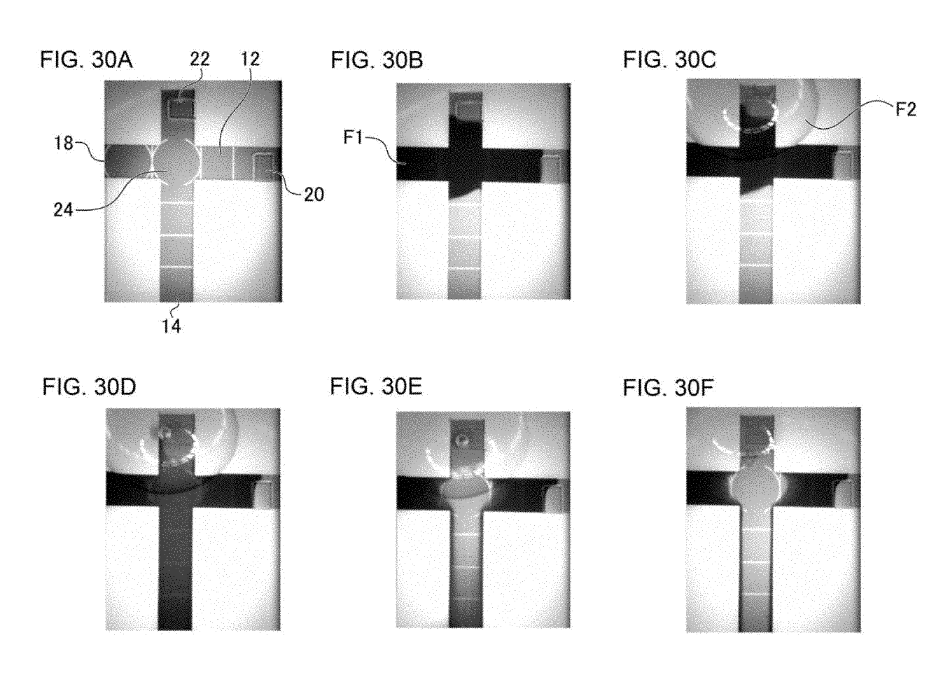

The inventor has actually confirmed the transfer of the first liquid and the second liquid using the sensor 1 according to the present embodiment. FIGS. 30A, 30B, 30C, 30D, 30E and 30F are photographs illustrating a state where the first liquid and the second liquid are transferred in the sensor 1 according to the ninth embodiment.

FIG. 30A is the photograph of the state of the sensor 1 before the first liquid F1 and the second liquid F2 are spotted to the first liquid supply port 18 and the second liquid supply port 22, respectively. Although the second exhaust hole 26 and the sealing member 28 are not illustrated, the second exhaust hole 26 is in the state of being closed by the sealing member 28.

FIG. 30B is the photograph of a state where the first liquid F1 is spotted to the first liquid supply port 18. When being spotted to the first liquid supply port 18, the first liquid F1 is drawn into the first part 12 due to the capillary phenomenon and is transferred to the first exhaust hole 20. Since the second liquid supply port 22 is also opened, a part of the first liquid F1 is drawn into the second liquid supply port 22 side from the analyte trap 24 (or the intersection part 416). Therefore, the analyte trap 24 may be provided not only at the intersection part 416 but also between the intersection part 416 and the second liquid supply port 22. Incidentally, the whole blood was used as the first liquid F1 in this experiment.

FIG. 30C is the photograph of a state where the second liquid F2 is spotted to the second liquid supply port 22. When the second liquid F2 is spotted to the second liquid supply port 22, the first liquid supply port 18 is closed by the first liquid F1. Incidentally, a wash solution was used as the second liquid F2 in this experiment.

FIGS. 30D 30E and 30F are the photographs of state changes over time after the second exhaust hole 26 is opened. Time has elapsed in the order of FIGS. 30D, 30E, and 30F. When the second exhaust hole 26 is opened, the second liquid F2 spotted to the second liquid supply port 22 is drawn into the second part 14 due to the capillary phenomenon as illustrated in FIGS. 30D and 30E. As a result, the first liquid F1 existing in the analyte trap 24 is pushed out by the second liquid F2.

Then, the second liquid F2 is further drawn into the second part 14 with the lapse of time as illustrated in FIG. 30F. Accordingly, the first liquid F1 and the second liquid F2 are transferred to the region C2a (see FIG. 26) of the second part 14. As a result, the first liquid F1 is almost completely removed from the analyte trap 24. In this experiment, it was confirmed that the first liquid F1 existing in the analyte trap 24 was almost completely replaced with the second liquid F2.

Therefore, it is possible to wash the composite 308 existing in the analyte trap 24 with the second liquid F2 if the composite 308 is formed by the antigen-antibody reaction among the solid-phase immobilized antibody 303, the antigen 304, and the labeled antibody 307. That is, it is possible to perform the B/F separation only by spotting of the first liquid F1 and the second liquid F2 and opening of the second exhaust hole 26 according to the sensor 1.

According to the sensor 1 according to the ninth embodiment described above, it is possible to perform the B/F separation and analyze and measure the analyte with high accuracy only by spotting of the first liquid F1 to the first liquid supply port 18, spotting of the second liquid F2 to the second liquid supply port 22, and opening of the second exhaust hole 26. Therefore, it is possible to achieve both the simplification of the device used for the analyte measurement and the ease of the analyte measurement. In addition, the first flow channel C1 and the second flow channel C2 intersect each other at the analyte trap 24. In other words, the first part 12 and the second part 14 intersect each other at the intersection part 416. It is possible to simplify the structure of the sensor 1 by adopting such a structure. Accordingly, it is possible to simplify the manufacturing process of the sensor 1, and to reduce the manufacturing cost of the sensor 1.

### Tenth Embodiment

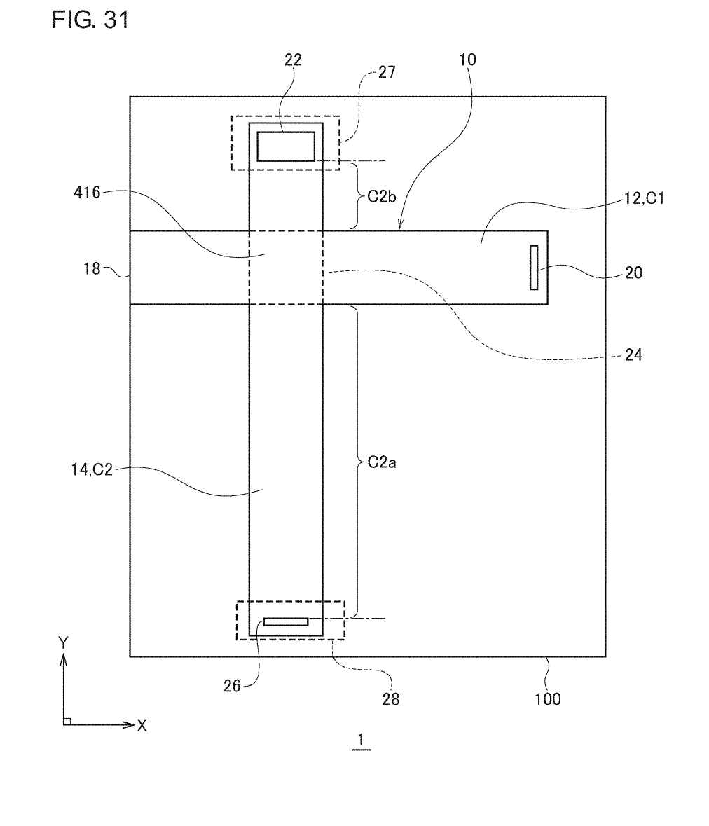

A sensor 1 according to the tenth embodiment has a configuration that is substantially common to the sensor 1 according to the ninth embodiment except that a second liquid supply port 22 can be closed. Hereinafter, the sensor 1 according to the present embodiment will be described focusing on a configuration different from the ninth embodiment. The same configuration as that of the ninth embodiment will be denoted by the same reference numerals, and the description thereof will be simplified or omitted as appropriate. FIG. 31 is a plan view schematically illustrating an internal structure of the sensor 1 according to the tenth embodiment when viewed from a cover substrate 106 side. For convenience of description, a first exhaust hole 20, the second liquid supply port 22, and a second exhaust hole 26 provided in the cover substrate 106 are also illustrated in FIG. 31.

In the sensor 1 according to the present embodiment, the second liquid supply port 22 can be switched from a closed state to an opened state. A second liquid F2 is drawn into a second flow channel C2 via the second liquid supply port 22 in the opened state.

The sensor 1 includes a sealing member 27 that closes the second liquid supply port 22. The sealing member 27 is configured using, for example, an adhesive tape or the like and is provided on a first main surface 106a of the cover substrate 106 so as to cover the second liquid supply port 22. It is possible to switch the second liquid supply port 22 from the closed state to the opened state by removing this sealing member 27 or by making a hole in the sealing member 27.

Incidentally, the second liquid supply port 22 may be closed as a material forming the cover substrate 106 is present inside the second liquid supply port 22. That is, the second liquid supply port 22 may be closed by a part of the cover substrate 106. The part of the cover substrate 106 positioned inside the second liquid supply port 22 corresponds to the sealing member 27. The part may be integrated with another part around the second liquid supply port 22. In this case, the second liquid supply port 22 is opened, for example, as the user forms a hole in a formation region of the second liquid supply port 22 of the cover substrate 106 at the timing of spotting the second liquid on the second liquid supply port 22. The cover substrate 106 is preferably subjected to processing to facilitate the formation of the second liquid supply port 22, such as making a thickness of a position where the second liquid supply port 22 is formed thinner than a thickness of the other region.

When the first liquid is supplied to the first liquid supply port 18 in a state where the first liquid supply port 18 and the first exhaust hole 20 are opened and the second liquid supply port 22 and the second exhaust hole 26 are closed, the first liquid is drawn into the first flow channel C1 from the first liquid supply port 18 along with discharge from the first exhaust hole 20. Then, the first liquid reaches the analyte trap 24 and further moves to the first exhaust hole 20.

When the second liquid is supplied to the second liquid supply port 22 in a state where the first liquid supply port 18 is closed and the second liquid supply port 22 and the second exhaust hole 26 are opened, the second liquid is drawn from the second liquid supply port 22 into the second flow channel C2 along with discharge from the second exhaust hole 26. Then, the second liquid passes through the analyte trap 24 and moves to the second exhaust hole 26 side. As the second liquid passes through the analyte trap 24, the first liquid can be removed from the analyte trap 24.

Next, a method of analyzing an analyte according to the present embodiment will be described. The analyte analysis method according to the present embodiment includes the following steps AII to CII.

Step AII: The first liquid F1 is supplied to the first liquid supply port 18 in a state where the second liquid supply port 22 and the second exhaust hole 26 are closed.

Step BII: The second liquid supply port 22 is opened to supply the second liquid F2 to the second liquid supply port 22 after Step AII.

Step CII: The second exhaust hole 26 is opened after the step AII and before, after, or simultaneously with the step BII.

In the step AII, the first liquid F1 is transferred to the analyte trap 24 due to a capillary phenomenon and is further transferred to the first exhaust hole 20. In addition, the second liquid F2 is transferred from the second liquid supply port 22 to the analyte trap 24 due to the capillary phenomenon in the step BII and the step CII. Then, the second liquid F2 passes through the analyte trap 24, and the first liquid F1 is removed from the analyte trap 24. The second liquid F2 having passed through the analyte trap 24 is further transferred to the second exhaust hole 26.

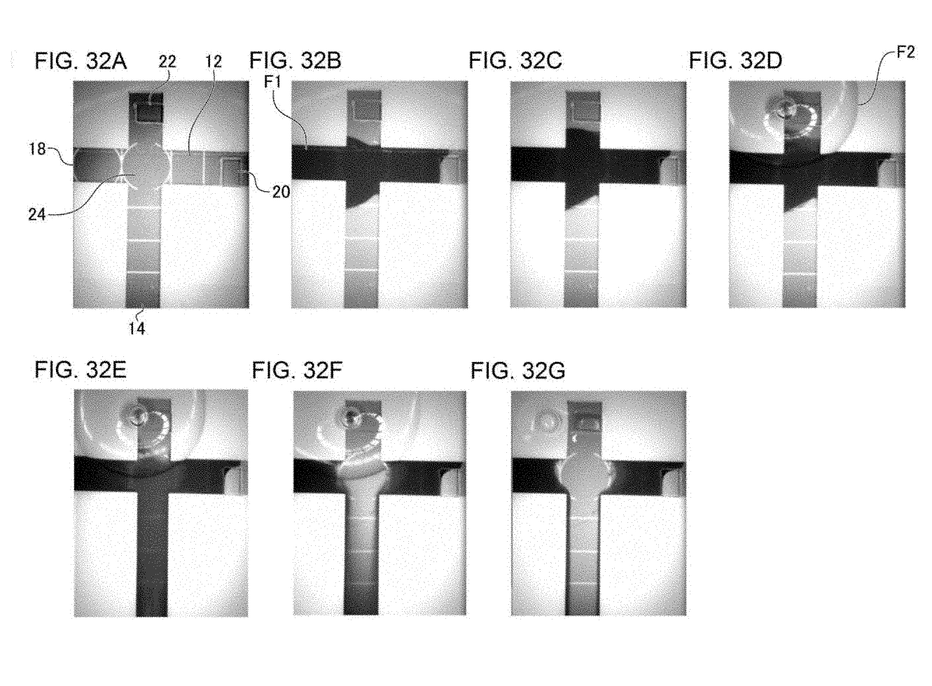

The inventor has actually confirmed the transfer of the first liquid and the second liquid using the sensor 1 according to the tenth embodiment. FIGS. 32A, 32B, 32C, 32D, 32E, 32F and 32G are photographs illustrating a state where the first liquid and the second liquid are transferred in the sensor 1 according to the tenth embodiment.

FIG. 32A is the photograph of the state of the sensor 1 before the first liquid F1 and the second liquid F2 are spotted to the first liquid supply port 18 and the second liquid supply port 22, respectively. Although the second exhaust hole 26 and the sealing member 28 are not illustrated, the second exhaust hole 26 is in the state of being closed by the sealing member 28. Although the sealing member 27 is not illustrated either, the second liquid supply port 22 is in the state of being closed by the sealing member 27.

FIGS. 32B and 32C are the photographs of changes over time after the first liquid F1 is spotted to the first liquid supply port 18. Time has elapsed in the order of FIGS. 32B and 32C. When being spotted to the first liquid supply port 18, the first liquid F1 is drawn into the first part 12 due to the capillary phenomenon and is transferred to the first exhaust hole 20 as illustrated in FIG. 32B. A part of the first liquid F1 is drawn into the second liquid supply port 22 side from the analyte trap 24 (or the intersection part 416) as illustrated in FIG. 32C. However, the amount of the first liquid F1 drawn into the second liquid supply port 22 side is smaller than that in the ninth embodiment (see FIG. 30B) since the second liquid supply port 22 is closed. Incidentally, the whole blood was used as the first liquid F1 in this experiment.

FIG. 32D is the photograph of a state where the second liquid F2 is spotted to the second liquid supply port 22. When the second liquid F2 is spotted to the second liquid supply port 22, the first liquid supply port 18 is closed by the first liquid F1. Incidentally, a wash solution was used as the second liquid F2 in this experiment.

FIGS. 32E, 32F and 32G are the photographs of state changes over time after the second exhaust hole 26 is opened. Time has elapsed in the order of FIGS. 32E, 32F, and 32G. When the second exhaust hole 26 is opened, the second liquid F2 spotted to the second liquid supply port 22 is drawn into the second part 14 due to the capillary phenomenon as illustrated in FIGS. 32E and 32F. As a result, the first liquid F1 existing in the analyte trap 24 is pushed out by the second liquid F2.

Then, the second liquid F2 is further drawn into the second part 14 with the lapse of time as illustrated in FIG. 32G. Accordingly, the first liquid F1 and the second liquid F2 are transferred to the region C2a (see FIG. 26) of the second part 14. As a result, the first liquid F1 is almost completely removed from the analyte trap 24. In this experiment, it was confirmed that the first liquid F1 existing in the analyte trap 24 was almost completely replaced with the second liquid F2.

Therefore, it is possible to wash the composite 308 existing in the analyte trap 24 with the second liquid F2 if the composite 308 is formed by the antigen-antibody reaction among the solid-phase immobilized antibody 303, the antigen 304, and the labeled antibody 307. That is, it is possible to perform the B/F separation only by spotting of the first liquid F1 and the second liquid F2 and opening of the second exhaust hole 26 according to the sensor 1.

According to the sensor 1 according to the tenth embodiment described above, it is possible to perform the B/F separation and analyze and measure the analyte with high accuracy only by spotting of the first liquid F1 to the first liquid supply port 18, opening of the second liquid supply port 22, spotting of the second liquid F2 to the second liquid supply port 22, and opening of the second exhaust hole 26. Therefore, it is possible to achieve both the simplification of the device used for the analyte measurement and the ease of the analyte measurement. In addition, the second liquid supply port 22 is closed when the first liquid F1 is supplied to the first flow channel C1 in the present embodiment. Thus, it is possible to suppress an increase in the amount of the first liquid F1 that is necessary to analyze the analyte.

### Eleventh Embodiment

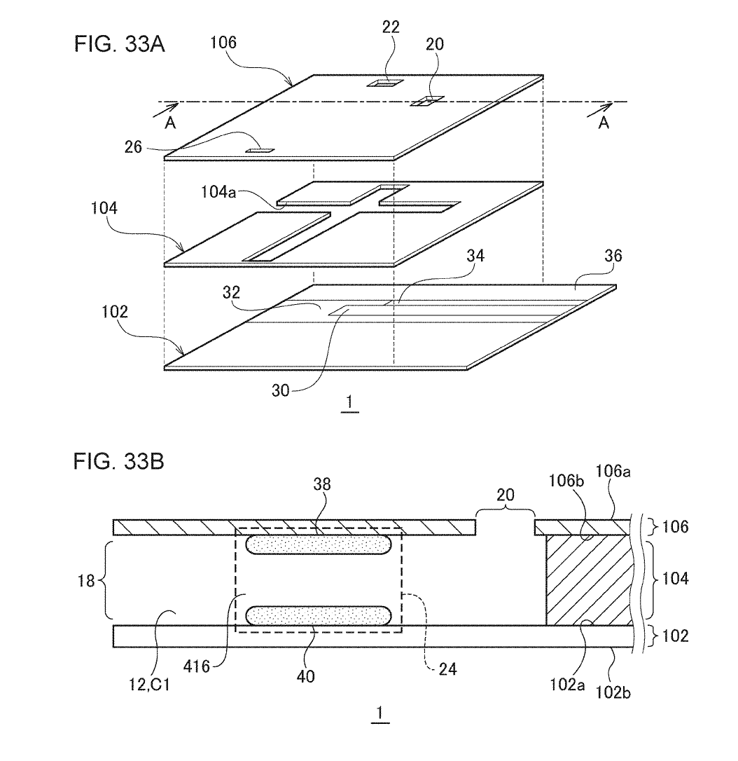

A sensor 1 according to the eleventh embodiment has a configuration that is substantially common to the sensor 1 according to the ninth embodiment except that a first reagent layer 38 and a second reagent layer 40 are provided in a first flow channel C1. Hereinafter, the sensor 1 according to the present embodiment will be described focusing on a configuration different from the ninth embodiment. The same configuration as that of the ninth embodiment will be denoted by the same reference numerals, and the description thereof will be simplified or omitted as appropriate. FIG. 33A is an exploded perspective view of the sensor 1 according to the eleventh embodiment. FIG. 33B is an enlarged view of the periphery of an analyte trap 24 in a cross section taken along a line A-A of FIG. 33A. For example, the sensor 1 according to the present embodiment includes a working electrode 30, a counter electrode 32, and a reference electrode 34 on a base substrate 102. Incidentally, the presence or absence of the electrode can be appropriately set in accordance with a measurement system to be adopted.

The sensor 1 includes the first reagent layer 38 and the second reagent layer 40 in a first flow channel C1. In the present embodiment, the first reagent layer 38 and the second reagent layer 40 are arranged in a space including the analyte trap 24 in the first flow channel C1, that is, in an intersection part 416. The first reagent layer 38 is fixed to a second main surface 106b of a cover substrate 106 and the second reagent layer 40 is fixed to a first main surface 102a of the base substrate 102. Incidentally, the first reagent layer 38 and the second reagent layer 40 may be arranged in a region other than the analyte trap 24 inside the first flow channel C1.

The first reagent layer 38 is, for example, a reagent layer containing a ruthenium complex-labeled antibody. The first reagent layer 38 is formed by, for example, dropping a predetermined amount of a ruthenium complex-labeled antibody solution onto the second main surface 106b of the cover substrate 106, and air-drying the solution. The second reagent layer 40 is, for example, a reagent layer containing a TnT antibody-labeled magnetic particle. The second reagent layer 40 is formed by, for example, dropping a predetermined amount of a TnT antibody-labeled magnetic particle solution onto the first main surface 102a of the base substrate 102, and air-drying the solution. The sensor 1 can be manufactured by forming the first reagent layer 38 and the second reagent layer 40 on the base substrate 102 and the cover substrate 106 before being bonded to each other, and then, bonding the base substrate 102, the spacer member 104, and the cover substrate 106 to each other.

Although the ruthenium complex-labeled antibody and the TnT antibody-labeled magnetic particle are contained in the separate reagent layers in the present embodiment, the invention is not limited to this configuration. For example, the sensor 1 may include only the first reagent layer 38 containing the ruthenium complex-labeled antibody and the TnT antibody-labeled magnetic particle. Alternatively, the sensor 1 may include only the second reagent layer 40 containing the ruthenium complex-labeled antibody and the TnT antibody-labeled magnetic particle. In addition, the first reagent layer 38 may contain the TnT antibody-labeled magnetic particle and the second reagent layer 40 may contain the ruthenium complex-labeled antibody. Both the first reagent layer 38 and the second reagent layer 40 may contain the TnT antibody-labeled magnetic particle and ruthenium complex-labeled antibody.

In addition, magnetic particles used as a solid phase 301 in the present embodiment, but TnT antibodies (primary antibodies 302) may be immobilized on a surface of a substrate forming the first flow channel C1, and a set of the immobilized TnT antibodies may be used as a reagent layer.

According to the sensor 1 of the present embodiment, it is possible to analyze and measure an analyte only by introducing an unprocessed specimen solution, such as blood, as a first liquid F1 into a first chamber 10 and then introducing a second liquid F2. That is, it is possible to omit pretreatment of a specimen solution such as blood. Thus, it is possible to more easily analyze and measure the analyte.

### Twelfth Embodiment

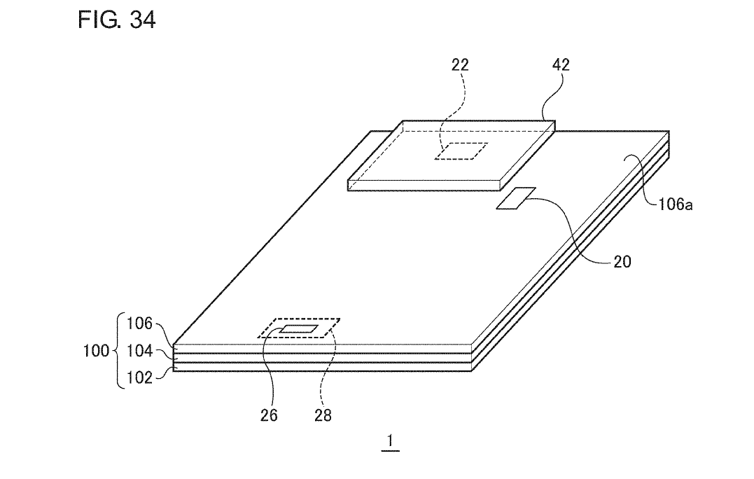

A sensor 1 according to the twelfth embodiment has a configuration that is substantially common to the sensor 1 according to the ninth embodiment except that a container 42 of a second liquid F2 is provided. Hereinafter, the sensor 1 according to the present embodiment will be described focusing on a configuration different from the ninth embodiment. The same configuration as that of the ninth embodiment will be denoted by the same reference numerals, and the description thereof will be simplified or omitted as appropriate. FIG. 34 is a perspective view illustrating a schematic structure of the sensor according to the twelfth embodiment.

The sensor 1 according to the present embodiment has a substrate 100 configured of a base substrate 102, a spacer member 104, and a cover substrate 106. The cover substrate 106 is provided with a first exhaust hole 20, a second liquid supply port 22, and a second exhaust hole 26 that communicate between a first chamber 10 (see FIG. 26) and the outside of the substrate 100. In addition, the sensor 1 is provided with a container 42 of a second liquid F2. The container 42 is, for example, a liquid holding bag, is arranged on an outer surface of the substrate 100, and is connected to the second liquid supply port 22. The container 42 is not particularly limited as long as being capable of holding a liquid, and examples thereof include an aluminum packaging material, a bag formed using a resin material such as polyethylene terephthalate (PET), polypropylene, and polyethylene, and the like.

The container 42 is fixed, for example, at a position, which covers the second liquid supply port 22, on a first main surface 106a of the cover substrate 106. In addition, the container 42 is fixed at a position that does not cover the first exhaust hole 20. Then, a needle is pierced through the container part 42 from the outside, for example, at a position where the container 42 and the second liquid supply port 22 overlap with each other in a stacking direction of the substrate 100 and the container 42, thereby forming a through-hole connecting the inside of the container 42 and the second liquid supply port 22 and a through-hole connecting the inside and the outside of the container 42. As a result, the second liquid F2 in the container 42 can be introduced into the first chamber 10 from the second liquid supply port 22 by a capillary force generated by opening of the second exhaust hole 26. Since the sensor 1 according to the present embodiment includes the container 42 of the second liquid F2, the analysis and measurement of the analyte can be further simplified.

### Thirteenth Embodiment

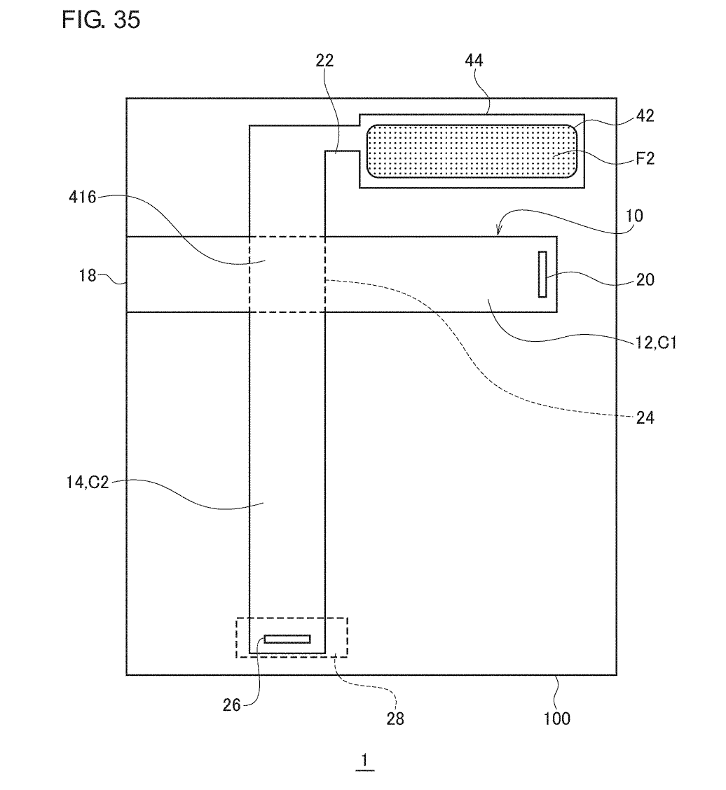

A sensor 1 according to the thirteenth embodiment has a configuration that is substantially common to the sensor 1 according to the ninth embodiment except that a second chamber 44 is provided and a first chamber 10 and the second chamber 44 communicate with each other. Hereinafter, the sensor 1 according to the present embodiment will be described focusing on a configuration different from the ninth embodiment. The same configuration as that of the ninth embodiment will be denoted by the same reference numerals, and the description thereof will be simplified or omitted as appropriate. FIG. 35 is a plan view schematically illustrating an internal structure of the sensor 1 according to the thirteenth embodiment when viewed from a cover substrate 106 side. For convenience of description, a first exhaust hole 20 and a second exhaust hole 26 provided in the cover substrate 106 are also illustrated in FIG. 35.

The sensor 1 according to the present embodiment is also common to the sensor 1 according to the twelfth embodiment in terms of holding a second liquid F2. However, the sensor 1 according to the present embodiment holds the second liquid F2 inside a substrate 100 while the sensor 1 according to the twelfth embodiment holds the second liquid F2 outside the substrate 100.

Specifically, the sensor 1 according to the present embodiment includes the second chamber 44, which accommodates the second liquid F2, inside the substrate 100. The second chamber 44 is defined by a first main surface 102a of a base substrate 102, a slit 104a of a spacer member 104, and a second main surface 106b of the cover substrate 106. Then, a second liquid supply port 22 communicates between the first chamber 10 and the second chamber 44. The second liquid supply port 22 is defined by the first main surface 102a of the base substrate 102, the slit 104a of the spacer member 104, and the second main surface 106b of the cover substrate 106. Inside the second chamber 44, a container 42 having a volume to be fitted in the second chamber 44 is arranged, and the second liquid F2 is accommodated in the container 42.

In such a configuration, for example, a through-hole connecting the inside of the container 42 and the inside of the second chamber 44 is formed by piercing a needle through the container 42 from the outside of the substrate 100. As a result, the second liquid F2 in the container 42 can be introduced into the first chamber 10 from the second liquid supply port 22 by a capillary force generated by opening of the second exhaust hole 26. Since the sensor 1 according to the present embodiment includes the second chamber 44 that accommodates the second liquid F2, the analysis and measurement of the analyte can be further simplified.

### Measurement Device

### Fourteenth Embodiment

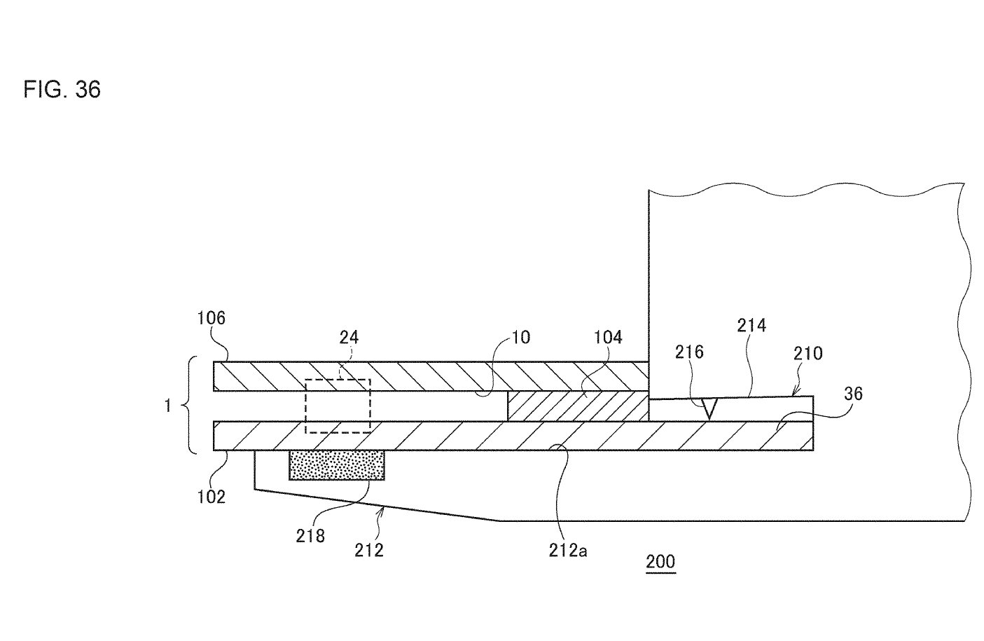

A measurement device using the sensor 1 according to each of the ninth to thirteenth embodiments described above will be described. FIG. 36 is an enlarged cross-sectional view illustrating the periphery of a sensor support in the measurement device. For example, FIG. 36 illustrates the sensor support of the measurement device used in the sensor 1 for an electrochemical signal measurement system. In addition, FIG. 24 illustrates the sensor support of the measurement device used in a measurement system in which a magnetic material is used as a solid phase 301. In addition, FIG. 36 illustrates a cross section of the sensor 1 taken along the X-axis direction at a predetermined position in the Y-axis direction in FIG. 26.

A measurement device 200 according to the present embodiment is a device that measures an analyte by detecting a signal acquired by analyte analysis using the sensor 1. The measurement device 200 includes a control unit 202, a memory 204, a display unit 206, an operation unit 208, and a measurement unit 210 (see FIG. 23), which is similar to the measurement device 200 according to the eighth embodiment. In addition, the measurement device 200 includes a sensor support 212 as illustrated in FIG. 36. Functions of the respective units are substantially similar to those of the measurement device 200 according to the eighth embodiment. Hereinafter, the measurement unit 210 will be described in detail.

### Measurement Unit

The measurement unit 210 detects and measures a signal generated by the sensor 1. The measurement unit 210 has various configurations necessary for measurement in accordance with a measurement system to be adopted in the sensor 1. Hereinafter, a configuration of the measurement unit 210 in accordance with each measurement system will be described.

### Electrochemical Signal Measurement System

When the measurement system of an analyte is an electrochemical signal measurement system, the measurement unit 210 includes a connector 214 as illustrated in FIG. 36. The connector 214 has an electrode 216. When the connection portion 36 of the sensor 1 is inserted into the connector 214, the electrode (see FIG. 28) provided in the sensor 1 is electrically connected to the electrode 216. The measurement unit 210 applies a predetermined voltage to the sensor 1 electrically connected via the electrode 216. As a result, the measurement unit 210 acquires a current value from the sensor 1.

Then, the measurement unit 210 transmits a signal indicating the obtained current value to the control unit 202. A conversion table in which a current value and an analyte concentration are associated with each other is stored in the memory 204. The control unit 202 measures the analyte concentration using the acquired current value and the conversion table. Then, the control unit 202 displays the obtained analyte concentration on the display unit 206, for example.

### Electrochemiluminescence Measurement System

When the analyte measurement system is an electrochemiluminescence measurement system, the measurement unit 210 includes the connector 214 and the electrode 216 similarly to the electrochemical signal measurement system. In addition, the measurement unit 210 includes a photodetector provided at a predetermined position. Examples of the photodetector include a photomultiplier tube (PMT), a charge coupled device (CCD), and the like. For example, the photodetector is arranged at a position facing the cover substrate 106 of the sensor 1 in a state where the sensor 1 is installed in the sensor support 212.

The measurement unit 210 applies a predetermined voltage to the sensor 1 electrically connected via the electrode 216. As a result, electrochemiluminescence occurs in the analyte trap 24 of the sensor 1. The measurement unit 210 detects this electrochemiluminescence by the photodetector. The measurement unit 210 transmits a signal indicating a light amount of detected electrochemiluminescence to the control unit 202. A conversion table in which a light amount and an analyte concentration are associated with each other is stored in the memory 204. The control unit 202 determines the analyte concentration using the acquired light amount and the conversion table. Then, the control unit 202 displays the obtained analyte concentration on the display unit 206, for example.

### Chemiluminescence/Bioluminescence Measurement System

When the analyte measurement system is a chemiluminescence/bioluminescence measurement system, the measurement unit 210 includes a photodetector similarly to the electrochemiluminescence measurement system. Since a configuration of the photodetector and a method of determining an analyte concentration by the control unit 202 are substantially similar to those of the electrochemiluminescence measurement system, the description thereof will be omitted.

### Fluorescence Measurement System

When the analyte measurement system is a fluorescence measurement system, the measurement unit 210 includes a light source and a photodetector provided at predetermined positions. The light source irradiates the sensor 1 with light having a predetermined wavelength to excite a fluorescent substance. Fluorescence occurs in the analyte trap 24 of the sensor 1 by the light emitted from the light source. The measurement unit 210 detects this fluorescence by the photodetector and transmits a signal indicating a light amount to the control unit 202. Since a configuration of the photodetector and a method of determining an analyte concentration by the control unit 202 are substantially similar to those of the electrochemiluminescence measurement system, the description thereof will be omitted.

### Absorbance Measurement System

When the analyte measurement system is an absorbance measurement system, the measurement unit 210 includes a light source and a light receiving element provided at predetermined positions. The light receiving element is configured using, for example, a photodiode or the like. The light source irradiates the sensor 1 with light having a predetermined wavelength. The light receiving element receives the light of the light source that has passed through the analyte trap 24 of the sensor 1. As a result, the measurement unit 210 acquires information on an absorbance.

The measurement unit 210 transmits a signal indicating the acquired information on the absorbance to the control unit 202. A conversion table in which an absorbance and an analyte concentration are associated with each other is stored in the memory 204. The control unit 202 determines the analyte concentration using the acquired absorbance and the conversion table. Then, the control unit 202 displays the obtained analyte concentration on the display unit 206, for example.

Next, a structure of the sensor support 212 will be described. The measurement device 200 has the sensor support 212 at a predetermined position as illustrated in FIG. 36. The sensor 1 is placed on the sensor support 212. For example, the sensor support 212 has a sensor mounting surface 212a. The sensor 1 is placed on the sensor mounting surface 212a such that the base substrate 102 is in contact with the sensor mounting surface 212a. The measurement unit 210 is adjacent to the sensor support 212. The sensor 1 is mounted on the sensor support 212, and the connection portion 36 is inserted into the connector 214.

In addition, the measurement device 200 includes a magnet 218 provided in the sensor support 212. The magnet 218 is arranged in the vicinity of the analyte trap 24 in a state where the sensor 1 is supported by the sensor support 212. For example, the magnet 218 is arranged at a position overlapping the analyte trap 24 when viewed from a direction in which the sensor support 212 and the sensor 1 are arrayed. When a magnetic material is used as the solid phase 301, that is, when the magnetic material is bonded to the analyte, the magnetic material is magnetized by the magnet 218 in the analyte trap 24. Accordingly, the analyte is captured in the analyte trap 24. As a result, it is possible to keep the analyte in the analyte trap 24 when the first liquid F1 existing in the analyte trap 24 is removed by the second liquid F2. Incidentally, a mechanism configured to perform perforating in the sealing member 27 or 28 or the container 42, or a mechanism configured to cause the second liquid F2 to be spotted to the second liquid supply port 22 may be provided in the sensor support 212 or the measurement unit 210.

Hereinafter, an analysis method that can be adopted by the above-described sensor 1 is the same as (1) and (2) exemplified in the eighth embodiment.

The present invention is not limited to the respective embodiments and modifications described above, and combinations of these embodiments and modifications and further modifications including various design changes or the like based on knowledge of those skilled in the art can be made. A new embodiment arising from such combinations or further modifications thereof is also included in the scope of the present invention.

Although both the first flow channel C1 and the second flow channel C2 has the linear shape as a whole in the above-described ninth to thirteenth embodiments, the present invention is not particularly limited to this configuration. Shapes of the first flow channel C1 and the second flow channel C2 (that is, the first part 12 and the second part 14) are not limited as long as intersecting each other at the analyte trap 24. The first flow channel C1 and the second flow channel C2 may have a shape other than the linear shape (for example, a curved shape or the like) as a whole, or partially include a portion having a shape other than the linear shape (for example, a curved shape or the like). Incidentally, it is more preferable that the first flow channel C1 and the second flow channel C2 have the linear shape as a whole from the viewpoints of simplification of a sensor structure and facilitation of flow of the first liquid F1 and the second liquid F2.

Incidentally, the following technical ideas are also included in this specification.

Item 1

A sensor for analyzing an analyte including:

a substrate;

a first chamber positioned inside the substrate;

a first liquid supply port which communicates between the first chamber and an outside of the substrate and through which a first liquid containing an analyte flows from the outside of the substrate to the first chamber;

an analyte trap positioned inside the first chamber and structured to capture the analyte in the first liquid;

a first exhaust hole which communicates between the first chamber and the outside of the substrate and through which a gas inside the first chamber flows to the outside of the substrate;

a first flow channel positioned inside the first chamber and connecting the first liquid supply port, the analyte trap, and the first exhaust hole;

a second liquid supply port which communicates between the first chamber and an outside of the first chamber and through which a second liquid containing a wash solution of the analyte trap flows from the outside of the first chamber to the first chamber;

a second exhaust hole which communicates between the first chamber and the outside of the substrate and is switchable from a closed state to an opened state, and through which the gas inside the first chamber flows to the outside of the substrate in the opened state; and

a second flow channel positioned inside the first chamber and connecting the second liquid supply port, the analyte trap, and the second exhaust hole,

wherein the first liquid supply port and the first exhaust hole are arranged with the analyte trap interposed therebetween in the first flow channel,

the second liquid supply port and the second exhaust hole are arranged with the analyte trap interposed therebetween in the second flow channel,

the first liquid is drawn into the first flow channel from the first liquid supply port along with discharge from the first exhaust hole and reaches the analyte trap in the closed state of the second exhaust hole, and

the second liquid is drawn into the second flow channel from the second liquid supply port along with discharge from the second exhaust hole, passes through the analyte trap, and removes the first liquid from the analyte trap in the opened state of the second exhaust hole.

Item 2

The sensor according to item 1, wherein

the first chamber includes a first part, a second part, and a coupler structured to couple the first part and the second part,

the first liquid supply port and the first exhaust hole communicate between the first part and the outside of the substrate,

the second liquid supply port communicates between the first part and the outside of the first chamber,

the second exhaust hole communicates between the second part and the outside of the substrate,

the first flow channel is arranged in the first part,

the second flow channel is arranged across the first part, the coupler, and the second part,

the first liquid supplied to the first liquid supply port moves through the first flow channel due to a capillary phenomenon and reaches the analyte trap in the closed state of the second exhaust hole, and

the second liquid supplied to the second liquid supply port moves through the second flow channel due to a capillary phenomenon and passes through the analyte trap, and reaches the second part via the coupler in the opened state of the second exhaust hole.

Item 3

The sensor according to item 2, wherein

the second liquid supply port communicates between the first chamber and the outside of the substrate, and also serves as the first exhaust hole.

Item 4

The sensor according to item 2, wherein

the second liquid supply port communicates between the first chamber and the outside of the substrate and is a separate body from the first exhaust hole,

the first exhaust hole is arranged between the second liquid supply port and the analyte trap in the second flow channel when viewed from a direction orthogonal to a main surface of the substrate, and

the second flow channel has a region that does not overlap with the first exhaust hole in a direction orthogonal to a center line of the second flow channel at a position overlapping with the first exhaust hole in a direction parallel to the center line when viewed from the direction orthogonal to the main surface of the substrate.

Item 5

The sensor according to item 2 or 3, wherein

the analyte trap is arranged between a position connected with the coupler in the first part and a position provided with the second liquid supply port, in a direction in which the second liquid flows in the second flow channel.

Item 6

The sensor according to item 2 or 4, wherein

the analyte trap is arranged between a position connected with the coupler in the first part and a position provided with the first exhaust hole, in a direction in which the second liquid flows in the second flow channel.

Item 7

The sensor according to any one of items 2 to 6, wherein

each number of the second part and the coupler is N (N is an integer of one or more), and

a sum of a volume of the N second parts and a volume of the N couplers is larger than a sum of a volume of the analyte trap in the first part and a volume between the first exhaust hole and the analyte trap in the first part.

Item 8

The sensor according to item 1 or 2 further including

a second chamber positioned inside the substrate and containing the second liquid,

wherein the second liquid supply port communicates between the first chamber and the second chamber.

Item 9

The sensor according to any one of items 1 to 7, further including

a container of the second liquid,

wherein the second liquid supply port communicates between the first chamber and the outside of the substrate, and

the container is arranged on an outer surface of the substrate and is connected to the second liquid supply port.

Item 10

The sensor according to item 1 or 2, wherein

the second liquid supply port communicates between the first chamber and the outside of the substrate, and also serves as the first liquid supply port.

Item 11

The sensor according to item 10, wherein

the first exhaust hole is arranged between the second exhaust hole and the analyte trap when viewed from a direction orthogonal to a main surface of the substrate, and

the second flow channel has a region that does not overlap with the first exhaust hole in a direction orthogonal to a center line of the second flow channel at a position overlapping with the first exhaust hole in a direction parallel to the center line when viewed from the direction orthogonal to the main surface of the substrate.

Item 12

The sensor according to item 1 or 2, wherein

the second liquid supply port communicates between the first chamber and the outside of the substrate and is a separate body from the first liquid supply port and the first exhaust hole,

the second liquid supply port and the second exhaust hole are arranged with the first liquid supply port, the analyte trap, and the first exhaust hole interposed therebetween when viewed from a direction orthogonal to a main surface of the substrate, and

the second flow channel has a region that does not overlap with the first liquid supply port in a direction orthogonal to a center line of the second flow channel at a position overlapping with the first liquid supply port in a direction parallel to the center line, and has a region that does not overlap with the first exhaust hole in the direction orthogonal to the center line at a position overlapping with the first exhaust hole in the direction parallel to the center line when viewed from the direction orthogonal to the main surface of the substrate.

Item 13

The sensor according to item 12, wherein

the first liquid supply port and the second exhaust hole are arranged on a same side, and the second liquid supply port and the first exhaust hole are arranged on a same side, with respect to the analyte trap.

Item 14

The sensor according to any one of items 1 to 13, wherein

the substrate includes a base substrate, a spacer member arranged on a surface of the base substrate, and a cover substrate arranged on a surface of the spacer member on a side opposite to the base substrate side,

the spacer member has a slit extending in a plane direction of the spacer member, and

the first chamber is formed by the surface of the base substrate, the surface of the cover substrate, and the slit.

Item 15

The sensor according to any one of items 1 to 14, further including

a sealing member structured to close the second exhaust hole.

Item 16

A measurement device including:

a sensor support on which the sensor according to any one of items 1 to 15 is placed; and

a magnet provided in the sensor support,

wherein a magnetic material is bound to the analyte, and

the analyte is captured as the magnetic material is magnetized by the magnet in the analyte trap.

Item 17

A method of analyzing an analyte using the sensor according to any one of items 1 to 15, the method including:

a step A of supplying the first liquid to the first liquid supply port and transferring the first liquid to the analyte trap using a capillary phenomenon in the closed state of the second exhaust hole;

a step B of supplying the second liquid to the second liquid supply port after the step A; and

a step C of opening the second exhaust hole after the step A and before, after, or simultaneously with the step B,

wherein the second liquid is transferred from the second liquid supply port to the analyte trap using a capillary phenomenon, and is caused to pass through the analyte trap to remove the first liquid from the analyte trap by the step B and the step C.

In addition, the following technical concept is also included in this specification.

Item 18

A sensor for analyzing an analyte including:

a substrate;

a first chamber positioned inside the substrate;

a first liquid supply port which communicates between the first chamber and an outside of the substrate and through which a first liquid containing an analyte flows from the outside of the substrate to the first chamber;

an analyte trap positioned inside the first chamber and structured to capture the analyte in the first liquid;

a first exhaust hole which communicates between the first chamber and the outside of the substrate and through which a gas inside the first chamber flows to the outside of the substrate;

a first flow channel positioned inside the first chamber and connecting the first liquid supply port, the analyte trap, and the first exhaust hole;

a second liquid supply port which communicates between the first chamber and an outside of the first chamber and through which a second liquid containing a wash solution of the analyte trap flows from the outside of the first chamber to the first chamber;

a second exhaust hole which communicates between the first chamber and the outside of the substrate and is switchable from a closed state to an opened state, and

through which the gas inside the first chamber flows to the outside of the substrate in the opened state; and

a second flow channel positioned inside the first chamber and connecting the second liquid supply port, the analyte trap, and the second exhaust hole,

wherein the first liquid supply port and the first exhaust hole are arranged with the analyte trap interposed therebetween in the first flow channel,

the second liquid supply port and the second exhaust hole are arranged with the analyte trap interposed therebetween in the second flow channel,

the first flow channel and the second flow channel intersect each other at the analyte step,

the first liquid is drawn into the first flow channel from the first liquid supply port along with discharge from the first exhaust hole and reaches the analyte trap in the closed state of the second exhaust hole, and

the second liquid is drawn into the second flow channel from the second liquid supply port along with discharge from the second exhaust hole, passes through the analyte trap, and removes the first liquid from the analyte trap in the opened state of the second exhaust hole.

Item 19

The sensor according to item 18, wherein

the first chamber includes a first part, a second part, and an intersection part between the first part and the second part,

the first liquid supply port and the first exhaust hole communicate between the first part and the outside of the substrate,

the second liquid supply port communicates between the second part and the outside of the first chamber,

the second exhaust hole communicates between the second part and the outside of the substrate,

the first flow channel is arranged in the first part,

the second flow channel is arranged in the second part,

the analyte trap is arranged in the intersection part,

the first liquid supplied to the first liquid supply port moves through the first flow channel due to a capillary phenomenon and reaches the analyte trap in the closed state of the second exhaust hole, and

the second liquid supplied to the second liquid supply port moves through the second flow channel due to a capillary phenomenon and passes through the analyte trap in the opened state of the second exhaust hole.

Item 20

The sensor according to item 18 or 19, wherein a volume of a region between the analyte trap and the second exhaust hole in the second flow channel is larger than a sum of a volume of a region between the second liquid supply port and the analyte trap in the second flow channel and a volume of the analyte trap in the second flow channel.

Item 21

The sensor according to any one of items 18 to 20, further including

a second chamber positioned inside the substrate and containing the second liquid,

wherein the second liquid supply port communicates between the first chamber and the second chamber.

Item 22

The sensor according to any one of items 18 to 20, wherein

the second liquid supply port communicates between the first chamber and the outside of the substrate.

Item 23

The sensor according to item 22, further including

a container of the second liquid,

wherein the container is arranged on an outer surface of the substrate and is connected to the second liquid supply port.

Item 24

The sensor according to any one of items 18 to 23, further including

a sealing member structured to close the second exhaust hole.

Item 25

The sensor according to any one of items 18 to 20, wherein

the second liquid supply port is switchable from a closed state to an opened state,

the first liquid is drawn into the first flow channel from the first liquid supply port and reaches the analyte trap in the closed states of the second liquid supply port and the second exhaust hole, and

the second liquid is drawn into the second flow channel from the second liquid supply port and passes through the analyte trap in the opened states of the second liquid supply port and the second exhaust hole.

Item 26

The sensor according to any one of items 18 to 25, wherein

the substrate includes a base substrate, a spacer member arranged on a surface of the base substrate, and a cover substrate arranged on a surface of the spacer member on a side opposite to the base substrate side,

the spacer member has a slit extending in a plane direction of the spacer member, and

the first chamber is formed by the surface of the base substrate, the surface of the cover substrate, and the slit.

Item 27

A measurement device including:

a sensor support on which the sensor according to any one of items 18 to 26 is placed; and

a magnet provided in the sensor support,

wherein a magnetic material is bound to the analyte, and

the analyte is captured as the magnetic material is magnetized by the magnet in the analyte trap.

Item 28

A method of analyzing an analyte using the sensor according to any one of items 18 to 24, the method including:

a step AI of supplying the first liquid to the first liquid supply port and transferring the first liquid to the analyte trap using a capillary phenomenon in the closed state of the second exhaust hole;

a step BI of supplying the second liquid to the second liquid supply port after the step AI; and

a step CI of opening the second exhaust hole after the step AI and before, after, or simultaneously with the step BI,

wherein the second liquid is transferred from the second liquid supply port to the analyte trap using a capillary phenomenon, and is caused to pass through the analyte trap to remove the first liquid from the analyte trap by the step BI and the step CI.

Item 29

A method of analyzing an analyte using the sensor according to item 25, the method including:

a step AII of supplying the first liquid to the first liquid supply port and transferring the first liquid to the analyte trap using a capillary phenomenon in the closed states of the second liquid supply port and the second exhaust hole;

a step BII of opening the second liquid supply port and supplying the second liquid to the second liquid supply port after the step AII; and

a step CII of opening the second exhaust hole after the step AII and before, after, or simultaneously with the step BII,

wherein the second liquid is transferred from the second liquid supply port to the analyte trap using a capillary phenomenon, and is caused to pass through the analyte trap to remove the first liquid from the analyte trap by the step BII and the step CII.

What is claimed is:

1. A sensor for analyzing an analyte comprising:
a substrate;
a first chamber positioned inside the substrate;
a first liquid supply port which communicates between the first chamber and an outside of the substrate and through which a first liquid containing an analyte flows from the outside of the substrate to the first chamber;
an analyte trap positioned inside the first chamber and structured to capture the analyte in the first liquid;
a first exhaust hole which communicates between the first chamber and the outside of the substrate and through which a gas inside the first chamber flows to the outside of the substrate;
a first flow channel positioned inside the first chamber and connecting the first liquid supply port, the analyte trap, and the first exhaust hole;
a second liquid supply port which communicates between the first chamber and an outside of the first chamber and through which a second liquid containing a wash solution of the analyte trap flows from the outside of the first chamber to the first chamber;
a second exhaust hole which communicates between the first chamber and the outside of the substrate and is switchable from a closed state to an opened state, and through which the gas inside the first chamber flows to the outside of the substrate in the opened state; and
a second flow channel positioned inside the first chamber and connecting the second liquid supply port, the analyte trap, and the second exhaust hole,
wherein the first liquid supply port and the first exhaust hole are arranged with the analyte trap interposed therebetween in the first flow channel,
the second liquid supply port and the second exhaust hole are arranged with the analyte trap interposed therebetween in the second flow channel,
the first flow channel and the second flow channel overlap with each other by a predetermined length in a region between the second liquid supply port and the analyte trap,
the first liquid is drawn into the first flow channel from the first liquid supply port due to a capillary phenomenon along with discharge from the first exhaust hole and reaches the analyte trap in the closed state of the second exhaust hole, and
the second liquid is drawn into the second flow channel from the second liquid supply port due to a capillary phenomenon along with discharge from the second exhaust hole, passes through the analyte trap, and removes the first liquid from the analyte trap in the opened state of the second exhaust hole.

2. The sensor according to claim 1, wherein
the second liquid supply port communicates between the first chamber and the outside of the substrate, and also serves as the first exhaust hole.

3. The sensor according to claim 1, wherein
the first exhaust hole is arranged between the second liquid supply port and analyte trap in the second flow channel,
the second liquid supply port communicates between the first chamber and the outside of the substrate, and
the second flow channel has a region that does not overlap with the first exhaust hole in a direction orthogonal to a center line of the second flow channel at a position overlapping with the first exhaust hole in a direction parallel to the center line when viewed from a direction orthogonal to a main surface of the substrate.

4. The sensor according to claim 1, wherein
the first chamber includes a first part, a second part, and a coupler structured to couple the first part and the second part,
the first liquid supply port and the first exhaust hole communicate between the first part and the outside of the substrate,
the second liquid supply port communicates between the first part and the outside of the first chamber,
the second exhaust hole communicates between the second part and the outside of the substrate,
the first flow channel is arranged in the first part,
the second flow channel is arranged across the first part, the coupler, and the second part,
the first liquid supplied to the first liquid supply port moves through the first flow channel due to a capillary phenomenon and reaches the analyte trap in the closed state of the second exhaust hole, and
the second liquid supplied to the second liquid supply port moves through the second flow channel due to a capillary phenomenon and passes through the analyte trap, and reaches the second part via the coupler in the opened state of the second exhaust hole.

5. The sensor according to claim 4, wherein
the analyte trap is arranged between a position connected with the coupler in the first part and a position provided with the first exhaust hole, in a direction in which the second liquid flows in the second flow channel.

6. The sensor according to claim 4, wherein
each number of the second part and the coupler is N (N is an integer of one or more), and
a sum of a volume of the N second parts and a volume of the N couplers is larger than a sum of a volume of the analyte trap in the first part and a volume between the first exhaust hole and the analyte trap in the first part.

7. The sensor according to claim 1, further comprising
a container of the second liquid,
wherein the second liquid supply port communicates between the first chamber and the outside of the substrate, and
the container is arranged on an outer surface of the substrate and is connected to the second liquid supply port.

8. The sensor according to claim 1, further comprising
a second chamber positioned inside the substrate and containing the second liquid,
wherein the second liquid supply port communicates between the first chamber and the second chamber.

9. The sensor according to claim 1, wherein
the second liquid supply port communicates between the first chamber and the outside of the substrate, and also serves as the first liquid supply port.

10. The sensor according to claim 9, wherein
the first exhaust hole is arranged between the second exhaust hole and the analyte trap in the second flow channel, and
the second flow channel has a region that does not overlap with the first exhaust hole in a direction orthogonal to a center line of the second flow channel at a position overlapping with the first exhaust hole in a direction parallel to the center line when viewed from a direction orthogonal to a main surface of the substrate.

11. The sensor according to claim 1, wherein
the second liquid supply port and the second exhaust hole are arranged with the first liquid supply port, the analyte trap, and the first exhaust hole interposed therebetween,
the second liquid supply port communicates between the first chamber and the outside of the substrate, and
the second flow channel has a region that does not overlap with the first liquid supply port in a direction orthogonal to a center line of the second flow channel at a position overlapping with the first liquid supply port in a direction parallel to the center line, and has a region that does not overlap with the first exhaust hole in the direction orthogonal to the center line at a position overlapping with the first exhaust hole in the direction parallel to the center line when viewed from a direction orthogonal to a main surface of the substrate.

12. The sensor according to claim 11, wherein
the first liquid supply port and the second exhaust hole are arranged on a same side, and the second liquid supply port and the first exhaust hole are arranged on a same side in the second flow channel, with respect to the analyte trap.

13. The sensor according to claim 1, wherein
the substrate includes a base substrate, a spacer member arranged on a surface of the base substrate, and a cover substrate arranged on a surface of the spacer member on a side opposite to the base substrate side,
the spacer member has a slit extending in a plane direction of the spacer member, and
the first chamber is formed by the surface of the base substrate, the surface of the cover substrate, and the slit.

14. The sensor according to claim 1, further comprising
a sealing member structured to close the second exhaust hole.

15. A method of analyzing an analyte using the sensor according to claim 1, the method comprising:
steps A and AI of supplying the first liquid to the first liquid supply port and transferring the first liquid to the analyte trap using a capillary phenomenon in the closed state of the second exhaust hole;
steps B and BI of supplying the second liquid to the second liquid supply port after the steps A and AI; and
steps C and CI of opening the second exhaust hole after the steps A and AI and before, after, or simultaneously with the steps B and BI,
wherein the second liquid is transferred from the second liquid supply port to the analyte trap using a capillary phenomenon, and is caused to pass through the analyte trap to remove the first liquid from the analyte trap by the steps B and BI and the steps C and CI.
